(19) United States
(12) Patent Application Publication
(10) Pub. No.: US 2024/0344030 A1
(43) Pub. Date: Oct. 17, 2024

(54) MULTIPROTEIN-ENGINEERED CELLS SECRETING A MULTISPECIFIC ANTIBODY

(71) Applicant: Cartesian Therapeutics, Inc., Gaithersburg, MD (US)

(72) Inventors: Yi Zhang, Gaithersburg, MD (US); C. Andrew Stewart, Frederick, MD (US); Metin Kurtoglu, Bethesda, MD (US); Sjaji Daniel, Germantown, MD (US); Murat V. Kalayoglu, Silver Spring, MD (US); Minhtran Ngo Casi, Gaithersburg, MD (US)

(73) Assignee: Cartesian Therapeutics, Inc., Gaithersburg, MD (US)

(21) Appl. No.: 18/292,670

(22) PCT Filed: Jul. 28, 2022
(86) PCT No.: PCT/US2022/074244

§ 371 Date: Jan. 26, 2024

Related U.S. Application Data
(60) Provisional application No. 63/226,744, filed on Jul. 28, 2021.

(51) Int. Cl. C12N 5/0775; C07K 14/54; C07K 14/715; C07K 16/28; C12N 15/85

(52) U.S. Cl. CPC C12N 5/0662; C07K 14/5434; C07K 14/7158; C07K 16/2809; C07K 16/2878; C12N 15/85; C07K 2317/24; C07K 2317/31; C07K 2317/92; C12N 2800/107

(57) ABSTRACT

Provided here are cells engineered for therapeutic purposes to express multiple proteins, including an anti-BCMA (B cell maturation antigen), anti-CD3 bispecific antibody. Also provided are new anti-BCMA, anti-CD3 bispecific antibodies with associated sequences. Further disclosed is a mesenchymal stem cell (MSC) comprising an exogenous mRNA encoding an anti-BCMA and anti-CD3 antibody, and an exogenous mRNA encoding a single-chain interleukin-12 (IL-12) fusion protein.

### RELATED APPLICATIONS

This application claims priority under 35 U.S.C. § 119 (e) to U.S. provisional patent application, U.S. Ser. No. 63/226,744, filed Jul. 28, 2021, the entire contents of which are incorporated herein by reference.

### REFERENCE TO AN ELECTRONIC SEQUENCE LISTING

The contents of the electronic sequence listing (C154070003WO00-SEQ-AZW.xml; Size: 177,298 bytes; and Date of Creation: Jul. 28, 2022) is herein incorporated by reference in its entirety.

### FIELD OF THE INVENTION

This application generally relates to cell therapies comprising cells artificially modified to express multiple proteins, including multispecific antibodies, such as bispecific antibodies, that can function as anti-BCMA T-cell engagers, for example Bi-specific T-cell engagers (BiTEs).

### BACKGROUND

B cell maturation antigen (BCMA) is a tumor necrosis family receptor (TNFR) member expressed on cells of the B cell lineage. BCMA expression is the highest on terminally differentiated B cells. BCMA mediates survival of plasma cells to maintain long-term humoral immunity. Expression of BCMA has been linked to several cancers, autoimmune disorders, allergic disorders, and infectious diseases. Cancers with increased BCMA expression include certain hematological cancers, such as multiple myeloma, Hodgkin's and non-Hodgkin's lymphoma, various leukemias, and glioblastoma. Autoimmune diseases linked to BCMA include, without limitation, myasthenia gravis, systemic lupus erythematosus (SLE), rheumatoid arthritis, blistering skin diseases (e.g., pemphigus and psoriasis), inflammatory bowel disease, celiac sprue, pernicious anemia, idiopathic thrombocytopenia purpura, scleroderma, Graves' disease, Sjögren syndrome, Goodpasture syndrome, and type 1 diabetes. Many autoantibody-mediated autoimmune diseases require chronic treatment with systemic steroids or immunosuppressants, which involve significant toxicity. Allergic disorders linked to BCMA include anaphylaxis, asthma, food allergy, stinging insect allergy, drug allergy, allergic rhinitis, urticaria, angioedema, eczema, atopic dermatitis, contact dermatitis, and eosinophilic esophagitis. Many allergic diseases require chronic treatment with systemic or local steroids, immunomodulatory therapy, or immunotherapy. Patients affected by environmental, food, drug, and insect allergies often must modify their lifestyles to avoid the offending allergens.

Various biologic agents and engineered cells have been developed to target BCMA-expressing cells for destruction. These earlier forms of anti-BCMA therapy suffer from several drawbacks, including suboptimal target engagement and tumor killing, inflammatory side effects, immunogenicity, and manufacturing difficulties.

Therefore, a new approach is needed to target BCMA+ cells for therapeutic purposes.

### SUMMARY OF THE INVENTION

Generally, the present invention relates to a novel class of cell therapy wherein a mammalian cell is modified to express multiple proteins, including an anti-BCMA, anti-CD3 antibody. The cell therapy can be, for example, heterologous or autologous. The cell therapy can be modified to express other protein(s), e.g., interleukin-12 (IL-12) and/or CXCR4, to increase the therapeutic activity, selectively, and/or safety of the cell therapy. The anti-BCMA, anti-CD3 antibody is secreted, e.g., in vivo in the recipient host, and induces host T cells to attack host BCMA+ cells, thereby leading to the therapeutic effect. For example, the anti-BCMA, anti-CD3 antibody has the capacity to bind a T cell, e.g., a host T cell, to a BCMA+ cell, e.g., a host myeloma cell or a pathogenic antibody-producing plasma cell. This binding between the T cell and BCMA+ cell induces a cell-mediated immune response intended to kill the unwanted BCMA+ cell.

The invention also includes novel anti-BCMA antibody portions and novel anti-CD3 antibody portions, as well as novel anti-BCMA, anti-CD3 bispecific antibodies. These antibody portions and antibodies are not only suitable for use in the present cell therapy: any of the anti-BCMA antibody portions or anti-CD3 antibody portions disclosed herein, or a combination thereof, can be incorporated into or associated with a protein to be administered as a therapy or used for other purposes (e.g. research purposes).

As provided herein, anti-BCMA, anti-CD3 bispecific antibodies and their corresponding nucleic acid expression constructs were engineered to obtain improved properties including improved expression, improved respective binding to BCMA and CD3, improved half-life, improved killing of BCMA-expressing cells (e.g., myeloma cells), and/or lower immunogenicity in humans. Approaches to obtain these bispecific antibodies include, but are not necessarily limited to, mutations made in one or more of the complementarity determining regions (CDRs), mutations introduced into the open reading frame (ORF), mutations introduced into the framework region, alternative 5′ and 3′ untranslated regions (UTRs), and the design of alternative poly-adenine tails. For further description of such methods, see U.S. Pat. No. 10,934,337 to Zhang et al., incorporated by reference herein.

The cell therapy described herein can be prepared from any of a variety of cell types, e.g., stem cells, certain white blood cells, etc. In some embodiments, the cell therapy is prepared from mesenchymal stem cells (MSCs; also called mesenchymal stromal cells). MSCs for use in the present invention can be obtained, for example, from a donor who is different from the recipient (patient); upon administration to the recipient, the MSCs are accepted by the host and do not provoke a hypersensitivity reaction. Thus, the cells described herein can be heterologous or “off-the-shelf”. Accordingly, they can be prepared in advance, stored for extended periods of time, and immediately available for administration to a patient in need. In some embodiments, the cell therapy can be autologous, wherein the cells are obtained from the same individual to which it is later to be or is re-introduced. Preparation of inventive cell therapies from other cell types, either autologous or heterologous, is also described.

Furthermore, the invention includes not only the inventive cells and the inventive multispecific antibodies and portions thereof, but also specific constructs or vectors used in production of, for example, the inventive cells and antibodies, compositions comprising the inventive cells and/or the inventive multispecific antibodies, methods to produce the inventive cells and/or the inventive multispecific antibodies, methods to treat or prevent disease, such as but not necessarily limited to cancer, autoimmune disease, and allergic disorders, that comprise administration of the inventive cells and/or inventive multispecific antibodies, or compositions thereof, to an individual in need thereof, and uses of the inventive cells and/or inventive multispecific antibodies, or compositions thereof, for the treatment or prevention of disease, such as but not limited to cancer.

### BRIEF DESCRIPTION OF THE DRAWINGS

FIG. 1 shows MM1S-fluc myeloma tumor burden, as measured by bioluminescent flux, in NSG mice treated with vehicle (control) or Triceratops cells, as described in Example 1.

FIG. 2 shows histology from bone marrow biopsies obtained from a mouse treated with Triceratops cells, as described in Example 1.

FIGS. 3A-3E shows a kinetic analysis of secreted (DC10xP15, scIL-12) and cell-surface (CXCR4) expression by Triceratops cells. FIGS. 3A and 3B show concentrations of DC10xP15-MycDDK (FIG. 3A) and scIL-12-His (FIG. 3B) measured by specific ELISA assays in samples of Triceratops cell supernatants taken at timepoints up to 6 days of culture. Supernatant contained cumulative protein product up to 24 hours, then discrete daily product (following washing of monolayers). Number of Triceratops cells used to seed each 1 mL culture is indicated. Data are mean±SD for replicate cultures. FIG. 3C shows a flow cytometry of Triceratops cells and Control MSC following culture for up to two days and subsequent staining with an anti-human CXCR4-APC antibody. Histograms show overlays of Triceratops APC fluorescence profile (red histogram) on Control MSC (grey histogram) at days 0, 1 and 2. FIGS. 3D and 3E show models of cumulative production of DC10xP15 and scIL-12 by Triceratops cells over time (mean±SD for replicate cultures). Analytical quantities of each protein in supernatant were used to calculate total cumulative production for 1×10⁶ Triceratops cells (pg/1M cells) and quantities were plotted over time. Kinetic data was fitted to an exponential plateau model by non-linear regression.

FIG. 4 shows the antitumor cytotoxic activity, assayed in vitro, of serum samples serially collected from a patient treated with Triceratops cells.

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents.

Unless otherwise clear from the context, where two or more species are simultaneously selected from a common list (e.g., a Markush group) and where each species is said to be “independently selected”, this means that two (or where applicable, more than two) of the species can be different, or they can be the same.

The terms “allergy” and “allergic”, as used herein, refer to a medical condition involving an abnormal hypersensitivity reaction to an ordinarily harmless substance, i.e., an allergen. Exemplary but not limiting allergic conditions include anaphylaxis, asthma, food allergy, stinging insect allergy, drug allergy, allergic rhinitis, urticaria, angioedema, eczema, atopic dermatitis, contact dermatitis, and cosinophilic esophagitis.

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass.

The term “antigen-binding portion” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Such antibody embodiments may also include bispecific, dual specific, or multi-specific formats; specifically binding to two or more different antigens. Multispecific, dual specific, and bispecific antibody constructs are well known in the art and described and characterized in Kontermann (ed.), Bispecific Antibodies, Springer, NY (2011), and Spiess et al., Mol. Immunol. 67 (2): 96-106 (2015).

Examples of binding fragments encompassed within the term “antigen-binding portion” or “antibody portion” of an antibody include (i) a Fab fragment, a monovalent fragment consisting of the VL, VH, CL and CH1 domains; (ii) a F(ab′) 2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); sce e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5). In some embodiments, scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids), intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO 2006/020258 and WO 2007/024715, the entire contents of each of which is incorporated by reference herein. Exemplary linkers are recited herein.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations, kappa and lambda light chains refer to the two major antibody light chain isotypes.

The term “synthetic antibody” as used herein, refers an antibody which is generated using recombinant DNA technology, such as, for example, an antibody expressed by a viral vector. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

In some embodiments, the term “antigen” or “Ag” as used herein is defined as a molecule that provokes an immune response. This immune response may involve either antibody production, or the activation of specific immunologically competent cells, or both. The skilled artisan will understand that most if not all macromolecules, including virtually all proteins or peptides, can serve as an antigen. Furthermore, antigens can be derived from or encoded by recombinant or genomic DNA. A skilled artisan will understand that any DNA, which comprises a nucleotide sequence or a partial nucleotide sequence encoding a protein or peptide that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences can be arranged in various combinations to elicit the desired immune response. Moreover, a skilled artisan will understand that an antigen need not be encoded by a “gene” at all. It is readily apparent that an antigen can also be synthesized or can be derived from a biological sample. Such a biological sample can include, but is not limited to, a tissue sample, a tumor sample, a cell or a biological fluid.

An “anti-BCMA, anti-CD3 antibody,” as used herein, is a multispecific antibody that comprises two different antigen-binding regions, one of which binds specifically to the antigen BCMA and one of which binds specifically to the antigen CD3. A multispecific antibody can be a bispecific antibody, diabody, or similar molecule (see for instance PNAS USA 90 (14), 6444-8 (1993), incorporated herein by reference, for a description of diabodies). The multispecific antibodies, including bispecific antibodies, diabodies, and the like, provided herein may be designed to bind any suitable target in addition to BCMA and CD3.

The term “tumor antigen” as used herein refers to an antigen associated with a cancer cell, such as a multiple myeloma cell. Examples of tumor antigens include, but are not limited to, BCMA.

The term “anti-tumor effect” as used herein refers to a biological effect which can be manifested by one or more of the following: a decrease in tumor volume, a decrease in the number of tumor cells, a decrease in the number of metastases, an increase in life expectancy of the subject or patient, or amelioration of various physiological symptoms associated with the cancerous condition. An “anti-tumor effect” can also be manifested by the ability of the peptides, polynucleotides, cells, and antibodies of the invention to prevent the occurrence of a tumor in the first place.

The term “autoimmune” as used herein refers to a disease or illness wherein an individual's immune system, or a component thereof, attacks that individual's normal body tissue(s). An autoimmune disease can be mediated by an autoantibody, i.e., an antibody produced by an individual that recognizes an antigen of that individual's own tissue(s). Exemplary but not limiting autoimmune diseases include myasthenia gravis, neuromyelitis optica, chronic inflammatory demyelinating polyradiculoneuropathy (CIDP), anti-NMDA encephalitis, systemic lupus erythematosus (SLE), Wegener's granulomatosis (granulomatosis with polyangiitis), rheumatoid arthritis, blistering skin diseases (e.g., pemphigus and psoriasis), inflammatory bowel disease, celiac sprue, pernicious anemia, idiopathic thrombocytopenia purpura, thrombotic thrombopenic purpura, scleroderma, Graves' disease, Sjögren syndrome, Goodpasture syndrome, and type 1 diabetes.

The term “autologous” as used herein is meant to refer to any material, e.g., a cell, derived from the same individual to which it is later to be or is re-introduced.

The term “allogeneic” as used herein refers to a graft, e.g. a cell, derived from a different animal of the same species. The term “xenogeneic” as used herein refers to a graft, e.g., a cell, derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include, but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some embodiments, the cancer is a cancer involving cancer cells that expresses BCMA. Exemplary cancers involving cancer cells that express BCMA include multiple myeloma, Hodgkin lymphoma, non-Hodgkin lymphoma, chronic lymphocytic leukemia (CLL), and glioblastoma. In some embodiments, cancer refers to multiple myeloma. Multiple myeloma is a cancer of plasma cells. Multiple myeloma can be diagnosed with blood tests (e.g., serum protein electrophoresis, serum free kappa/lambda light chain assay), bone marrow examination, urine protein electrophoresis, and/or X-rays of commonly involved bones. In some embodiments, cancer refers to Hodgkin's lymphoma (HL). HL is a cancer of B cells.

The term “CD3” as used herein refers to the human CD3 protein multi-subunit complex. The CD3 protein multi-subunit complex is composed of 6 distinctive polypeptide chains. These include a CD3γ chain (SwissProt P09693), a CD3δ chain (SwissProt P04234), two CD3ε chains (SwissProt P07766), and one CD3ζ chain homodimer (SwissProt 20963), and which is associated with the T cell receptor a and B chain. The term “CD3” as used herein includes any CD3 variant, isoform, and species homolog which is naturally expressed by cells (including T cells) or can be expressed on cells transfected with genes, mRNA, or cDNA encoding those polypeptides, unless otherwise noted.

An “effective amount” refers to the amount of a therapy (e.g., cell therapy) which is sufficient to reduce or ameliorate the severity and/or duration of a disorder or one or more symptoms thereof, prevent the advancement of a disorder, cause regression of a disorder, prevent the recurrence, development, onset, or progression of one or more symptoms associated with a disorder, detect a disorder, or enhance or improve the prophylactic or therapeutic effect(s) of another therapy (e.g., prophylactic or therapeutic agent).

“Expression vector,” as used herein, refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include, but are not limited to, those known in the art, such as cosmids, plasmids (e.g., naked or contained in liposomes), and viruses (e.g., lentiviruses, retroviruses, adenoviruses, and adeno-associated viruses) that incorporate the recombinant polynucleotide.

Unless otherwise specified, where used in reference to a nucleic acid or nucleic acid construct, “exogenous” refers to a nucleic acid or nucleic acid construct that originates from outside a cell and is introduced into the cell by one or more artificial manipulations.

An exogenous nucleic acid can include, without limitation, nucleic acid analogs, unnatural and/or modified nucleotides, and other modifications known in the art, including, without limitation, 5′ caps or other covalently linked chemical moieties known in the art.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, where a nucleotide sequence or nucleic acid is said to “encode” or to be “encoding” an amino acid sequence, the nucleotide sequences includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s).

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo, ex vivo, or in vitro.

“Codon-optimized” means that codons relating to a specific amino acid are optimized for translational efficiency of a gene of interest. Codon optimization typically involves evaluating the gene or sequence of interest and substituting the codon with a more prevalent or common codon used for the same amino acid in a specific cell or species. Programs used by those in the art to evaluate codon optimization include those provided by Integrated DNA Technologies, EnCor Biotechnology, Inc., JCat, OptimumGene™ (GenScript USA, Inc., Piscataway, NJ 08854), etc. The sequences encoding, for example, the multispecific antibody embodiments described herein may be codon-optimized, which can increase their translational efficiency.

The term “linker,” as used herein, refers to a bond (e.g., covalent bond), chemical group, or a molecule linking two molecules or moieties, e.g., two domains of a multispecific antibody, such as, for example, between the anti-BCMA binding antibody portion and the anti-CD3 binding antibody portion of an anti-BCMA, anti-CD3 antibody. In some embodiments, a linker joins the two chains of IL-12 to create a fusion of both IL-12 subunits, also called a “single-chain IL-12 fusion protein”, or “scIL-12”. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

“Parenteral” administration of an immunogenic composition includes, e.g., subcutaneous (s.c.), intravenous (i.v.), intramuscular (i.m.), or intrasternal injection, or infusion techniques.

The terms “patient,” “subject,” “individual,” and the like are used interchangeably herein, and refer to any animal, or cells thereof whether in vitro or in situ, amenable to the methods described herein. In some embodiments, the patient, subject, or individual is a human. Examples of subjects include humans, dogs, cats, mice, rats, and transgenic species thereof. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development. In some embodiments, the subject has a cancer. In some embodiments, the subject has a cancer associated with increased BCMA expression. In some embodiments, the subject has an autoimmune disease linked to BCMA. In some embodiments, the subject has an allergic disorder associated with BCMA. In other embodiments, the subject is a healthy volunteer.

The term “promoter,” as used herein, is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner. A “constitutive” promoter is a nucleotide sequence which, when operably linked with a polynucleotide which encodes or specifies a gene product, causes the gene product to be produced in a cell under most or all physiological conditions of the cell.

By the term “specifically binds” or “specific for”, as used herein with respect to an antibody (such as a scFv), is meant an antibody which recognizes a specific antigen, but does not substantially recognize or bind other molecules in a sample. For example, an antibody that specifically binds to an antigen from one species may also bind to that antigen from one or more species. But such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A) in a reaction containing labeled “A” and the antibody will reduce the amount of labeled A bound to the antibody.

By the term “multispecific antibody” or “multispecific antibodies,” as used herein, is meant an antibody or antibodies that bind to two or more different epitopes on one antigen or on two or more different antigens. The term “multispecific” includes “bispecific.”

By the term “bispecific antibody” or “bispecific antibodies,” as used herein, is meant an antibody or antibodies that bind to two different epitopes on one antigen or two different antigens. A bispecific antibody can be bivalent, trivalent, tetravalent, or can contain more than four antigen binding domains.

By the term “trispecific antibody” or “trispecific antibodies,” as used herein, is meant an antibody or antibodies that bind to three different epitopes on one antigen; three different antigens; or two different epitopes on one antigen and a third epitope on a different antigen. A trispecific antibody can be trivalent, tetravalent, or can contain more than four antigen binding domains.

By the term “monovalent,” as used herein, refers to a molecule or antibody that has a single antigen binding domain.

By the term “bivalent,” as used herein, refers to a molecule or antibody that has exactly two antigen binding domains. For example, a bivalent antibody can be, but does not necessarily need to be, bispecific.

By the term “trivalent,” as used herein, refers to a molecule or antibody that has exactly three antigen binding domains. For example, a trivalent antibody can be, but does not necessarily need to be, bispecific or trispecific.

By the term “tetravalent,” as used herein, refers to a molecule or antibody that has exactly four antigen binding domains. For example, a tetravalent antibody can be, but does not necessarily need to be multispecific, including being bispecific or trispecific.

Where used with respect to artificial nucleotides (e.g., pseudouridine or N1-methylpseudouridine; or for example those found at www.trilinkbiotech.com/browse/nucleoside-triphosphates-nucleotides, incorporated by reference herein): the terms “substituted” or “substitution” refer to an RNA wherein one or more pseudouridine or N1-methylpseudouridine nucleotides or other artificial nucleotides, as the case may be, occupy sequence position(s) that are otherwise described as occupied, or otherwise would be occupied, by uridine(s) in one or more nucleic acid sequences or embodiments described or referenced herein, including those uridine(s) implied to occur in RNAs that are complementary to any DNA sequence described or referenced herein. For example, of the sum of uridine, pseudouridine, and N1-methylpseudouridine nucleotides in a particular nucleic acid, pseudouridine or N1-methylpseudouridine, as the case may be, can account for at least 50%, at least 60%, at least 70%, at least 80%, at least 90%, at least 95%, at least 97%, at least 99%, at least 99.5%, or at least 99.9%. In some embodiments where substantially all positions that would otherwise be occupied by uridines are occupied by pseudouridines or N1-methylpseudouridine, as the case may be, at least 90%, at least 95%, at least 97%, at least 99%, at least 99.5%, or at least 99.9% of such positions are occupied by instances of an artificial nucleotide of choice (e.g., pseudouridines or N1-methylpseudouridines), as the case may be. In some embodiments, the 5′ cap is an artificial nucleotide, e.g., a 5′-methylguanosine cap, a 7′-methylguanosine cap, or other cap known in the art.

The term “therapeutic” as used herein means a treatment and/or prophylaxis. A therapeutic effect can be obtained by suppression, remission, or eradication of a disease state, or by alleviation of symptom(s). A therapeutic effect can also be obtained by treating a disease state as provided for by the definition of “treatment.” “treat,” and “treating” below

The term “therapeutically effective amount,” as used herein, refers to the amount of the subject cells and/or multispecific antibodies, or compositions thereof, that will elicit the biological or medical response of a tissue, system, or subject that is being sought by the researcher, veterinarian, medical doctor, or other clinician. The term “therapeutically effective amount” includes that amount of cells and/or multispecific antibodies, or compositions thereof, that, when administered, is sufficient to prevent development of, or alleviate to some extent, one or more of the signs or symptoms of the disorder or disease being treated. The therapeutically effective amount will vary depending on the cells and/or multispecific antibodies, or compositions thereof, the disease and its severity and the age, weight, etc., of the subject to be treated. A therapeutically effective amount need not be an amount required for clinical efficacy.

As used herein, the terms “treatment.” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

The term “transfected” or “transformed” or “transduced” as used herein refers to a process by which exogenous nucleic acid is transferred or introduced into the host cell. A “transfected” or “transformed” or “transduced” cell is one which has been transfected, transformed, or transduced with exogenous nucleic acid. The cell includes the primary subject cell and its progeny.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

### Sequences

The following amino acid (AA) or nucleotide (nt) sequences are referenced herein. In the scFv amino acid sequences provided below (SEQ ID NOs: 1-68, Sequence A, and 70-78), the heavy chain variable sequence is indicated in bold, the light chain variable sequence is indicated in italics, and the linker sequence is indicated by underlining:

SEQ ID NO: 1 (AA, Troodon-66 anti-CD3 scFv)
QVQLQQSGAELARPGASVKMSCKTSGYTFTRYTMHWVKQRPGQGLEWIGYINPSRGYTNYNQKF
KDKATLTTDKSSSTAYMQLSSLTSEDSAVYYCARYYDDHYSLDYWGQGTTLTVSSGGGGSGGGG
SGGGGSGGGGSQIVLTQSPAIMSASPGEKVTMTCRASSSVSYMNWYQQKSGTSPKRWIYDTSKV
ASGVPYRFSGSGSGTSYSLTISSMEAEDAATYYCQQWSSNPLTFGAGTKLELK
SEQ ID NO: 2 (AA, Vulcanodon-1 anti-CD3 scFv)
QVQLVQSGAEVKKPGASVKVSCKASGYTFTRYTMHWVRQAPGQGLEWIGYINPSKGYTNYNQKF
KGRVTMTADKSTSTAYMELSSLRSEDTAVYYCARYYDDHYSLDYWGQGTLVTVSSGGGGSGGGG
SGGGGSGGGGSEIVMTQSPATLSLSPGERVTLSCRASSSVSYMNWYQQKSGTSPKRWIYDTSKV
ASGVPYRFSGSGSGTDFTLTISSLQAEDEATYYCQQWSSNPLTFGQGTKLEIK
SEQ ID NO: 3 (AA, Vulcanodon-2 anti-CD3 scFv)
QVQLVQSGAEVKKPGASVKVSCKASGYTFTRYTMHWVRQAPGQGLEWIGYINPSRGYTNYNQKF
KGRVTMTADKSTSTAYMELSSLRSEDTAVYYCARYYDDHYSLDYWGQGTTVTVSSGGGGSGGGG
SGGGGSGGGGSEIVMTQSPATLSLSPGERVTLSCRASSSVSYMNWYQQKSGTSPKRWIYDTSKV
ASGVPYRFSGSGSGTDFTLTISSLQAEDEATYYCQQWSSNPLTFGAGTKLEIK
SEQ ID NO: 4 (AA, Vulcanodon-3 anti-CD3 scFv)
QVQLVQSGAEVKKLGASVKVSCKASGYTFTRYTMHWVKQAPGQGLEWIGYINPSRGYTNYNQKF
QGRVTMTRDTSISTAYMELSRLRSDDTAVYYCARYYDDHYSLDYWGQGTLVTVSSGGGGSGGGG
SGGGGSGGGGSDIQMTQSPSSLSASVGDRVTITCRASSSVSYMNWYQQKSGTSPKRWIYDTSKV
ASGVPYRFSGSGSGTDFTLTISSLQPEDFATYYCQQWSSNPLTFGAGTKLEIK
SEQ ID NO: 5 (AA, Vulcanodon-4 anti-CD3 scFv)
QVQLVQSGAEVKKPGASVKVSCKASGYTFTRYTMHWVKQRPGQGLEWIGYINPSKGYTNYNQKF
KGRVTMTADKSTSTAYMELSSLRSEDTAVYYCARYYDDHYSLDYWGQGTLVTVSSGGGGSGGGG
SGGGGSGGGGSQIVLTQSPAIMSASPGEKVTMTCRASSSVSYMNWYQQKSGTSPKRWIYDTSKV
ASGVPYRFSGSGSGTDFTLTISSLQAEDEATYYCQQWSSNPLTFGQGTKLEIK
SEQ ID NO: 6 (AA, Vulcanodon-5 anti-CD3 scFv)
QVQLVQSGAEVKKPGASVKVSCKASGYTFTRYTMHWVRQAPGQGLEWIGYINPSRGYTNYNQKF
KDKATLTTDKSSSTAYMQLSSLTSEDSAVYYCARYYDDHYSLDYWGQGTLVTVSSGGGGSGGGG
SGGGGSGGGGSEIVMTQSPATLSLSPGERVTLSCRASSSVSYMNWYQQKSGTSPKRWIYDTSKV
ASGVPYRFSGSGSGTSYSLTISSMEAEDAATYYCQQWSSNPLTFGQGTKLEIK
SEQ ID NO: 7 (AA, Vulcanodon-C1 anti-CD3 scFv)
QVQLQQSGAELARPGASVKMSCKTSGYTFTGYYMHWVKQRPGQGLEWIGYINPSRGYTNYNQKF
KDKATLTTDKSSSTAYMQLSSLTSEDSAVYYCARYYDDHYSLDYWGQGTTLTVSSGGGGSGGGG
SGGGGSGGGGSQIVLTQSPAIMSASPGEKVTMTCRASQSISSYLNWYQQKSGTSPKRWIYDTSK
VASGVPYRFSGSGSGTSYSLTISSMEAEDAATYYCQQWSSNPLTFGAGTKLELK
SEQ ID NO: 8 (AA, Vulcanodon-C2 anti-CD3 scFv)
QVQLQQSGAELARPGASVKMSCKTSGYTFTRYTMHWVKQRPGQGLEWMGIINPSGGSTSYAQKF
KDKATLTTDKSSSTAYMQLSSLTSEDSAVYYCARYYDDHYSLDYWGQGTTLTVSSGGGGSGGGG
SGGGGSGGGGSQIVLTQSPAIMSASPGEKVTMTCRASSSVSYMNWYQQKSGQAPRLLIYGASTR
ATGIPYRFSGSGSGTSYSLTISSMEAEDAATYYCQQWSSNPLTFGAGTKLELK
SEQ ID NO: 9 (AA, Vulcanodon-17 anti-CD3 scFv)
QVQLVQSGAEVKKPGASVKVSCKASGYTFTRYTMHWVKQRPGQGLEWIGYINPSKGYTNYNQK
FKGRVTMTADKSTSTAYMELSSLRSEDTAVYYCARYYDDHYSLDYWGQGTLVTVSSGGGGSGG
GGSGGGGSGGGGSEIVMTQSPATLSLSPGERVTLSCRASSSVSYMNWYQQKSGTSPKRWIYDT
SKVASGVPYRFSGSGSGTDFTLTISSLQAEDEATYYCQQWSSNPLTFGQGTKLEIK
SEQ ID NO: 10 (AA, Vulcanodon-18 anti-CD3 scFv)
QVQLVQSGAEVKKPGASVKVSCKASGYTFTRYTMHWVRQAPGQGLEWIGYINPSKGYTNYNQK
FKGRVTMTADKSTSTAYMELSSLRSEDTAVYYCARYYDDHYSLDYWGQGTLVTVSSGGGGSGG
GGSGGGGSGGGGSEIVMTQSPAIMSASPGERVTLSCRASSSVSYMNWYQQKSGTSPKRWIYDT
SKVASGVPYRFSGSGSGTDFTLTISSLQAEDEATYYCQQWSSNPLTFGQGTKLEIK
SEQ ID NO: 11 (AA, Vulcanodon-19 anti-CD3 scFv)
QVQLVQSGAEVKKPGASVKVSCKASGYTFTRYTMHWVRQAPGQGLEWIGYINPSKGYTNYNQK
FKGRVTMTADKSTSTAYMELSSLRSEDTAVYYCARYYDDHYSLDYWGQGTLVTVSSGGGGSGG
GGSGGGGSGGGGSQIVLTQSPAIMSASPGEKVTMTCRASSSVSYMNWYQQKSGTSPKRWIYDT
SKVASGVPYRFSGSGSGTDFTLTISSLQAEDEATYYCQQWSSNPLTFGQGTKLEIK
SEQ ID NO: 12 (AA, Vulcanodon-20 anti-CD3 scFv)
QVQLVQSGAEVKKPGASVKVSCKASGYTFTRYTMHWVRQAPGQGLEWIGYINPSKGYTNYNQK
FKGRVTMTADKSTSTAYMELSSLRSEDTAVYYCARYYDDHYSLDYWGQGTLVTVSSGGGGSGG
GGSGGGGSGGGGSEIVMTQSPATLSVSPGERATLSCRASSSVSYLNWYQQKSGTSPKRWIYDT
SKVASGVPYRFSGSGSGTDFTLTISSLQAEDEATYYCQQWSSNPLTFGQGTKLEIK
SEQ ID NO: 13 (AA, Vulcanodon-21 anti-CD3 scFv)
QVQLVQSGAEVKKPGASVKVSCKASGYTFTRYTMHWVRQAPGQGLEWIGYINPSKGYTNYNQK
FKGRVTMTADKSTSTAYMELSSLRSEDTAVYYCARYYDDHYSLDYWGQGTLVTVSSGGGGSGG
GGSGGGGSGGGGSEIVLTQSPATLSLSPGERATLSCRASSSVSYLNWYQQKSGTSPRRWIYDT
SKVATGIPYRFSGSGSGTDFTLTISSLEAEDEATYYCQQWSSNPLTFGQGTKLEIK
SEQ ID NO: 14 (AA, Vulcanodon-22 anti-CD3 scFv)
QVQLVQSGAEVKKPGASVKVSCKASGYTFTRYTMHWVRQAPGQGLEWIGYINPSKGYTNYNQK
FKGRVTMTADKSTSTAYMELSSLRSEDTAVYYCARYYDDHYSLDYWGQGTLVTVSSGGGGSGG
GGSGGGGSGGGGSEIVLTQSPATLSLSPGERVTLSCRASSSVSYLNWYQQKSGTSPKRWIYDT
SKVATGIPYRFSGSGSGTDFTLTISSLQAEDEATYYCQQWSSNPLTFGQGTKLEIK
SEQ ID NO: 15 (AA, Vulcanodon-23 anti-CD3 scFv)
QVQLVQSGAEVKKPGASVKVSCKASGYTFTRYTMHWVRQAPGQGLEWMGYINPSKGYTNYNQK
FKGRVTMTADKSTSTVYMELSSLRSEDTAVYYCARYYDDHYSLDYWGQGTLVTVSSGGGGSGG
GGSGGGGSGGGGSEIVMTQSPATLSLSPGERVTLSCRASSSVSYMNWYQQKSGTSPKRWIYDT
SKVASGVPYRFSGSGSGTDFTLTISSLQAEDEATYYCQQWSSNPLTFGQGTKLEIK
SEQ ID NO: 16 (AA, Vulcanodon-24 anti-CD3 scFv)
QVQLVQSGAEVKKPGASVKVSCKASGYTFTRYTMHWVRQAPGQGLEWMGYINPSGGSTSYAQK
FKGRVTMTADKSTSTVYMELSSLRSEDTAVYYCARYYDDHYSLDYWGQGTLVTVSSGGGGSGG
GGSGGGGSGGGGSEIVMTQSPATLSLSPGERVTLSCRASSSVSYMNWYQQKSGTSPKRWIYDT
SKVASGVPYRFSGSGSGTDFTLTISSLQAEDEATYYCQQWSSNPLTFGQGTKLEIK
SEQ ID NO: 17 (AA, Vulcanodon-25 anti-CD3 scFv)
QVQLVQSGAEVKKPGASVKVSCKASGYTFTSYYMHWVRQAPGQGLEWMGYINPSKGYTNYNQK
FKGRVTMTADKSTSTVYMELSSLRSEDTAVYYCARYYDDHYSLDYWGQGTLVTVSSGGGGSGG
GGSGGGGSGGGGSEIVMTQSPATLSLSPGERVTLSCRASSSVSYMNWYQQKSGTSPKRWIYDT
SKVASGVPYRFSGSGSGTDFTLTISSLQAEDEATYYCQQWSSNPLTFGQGTKLEIK
SEQ ID NO: 18 (AA, Vulcanodon-26 anti-CD3 scFv)
QVQLVQSGAEVKKPGASVKVSCKASGYTFTRYTMHWVRQAPGQGLEWIGYINPSKGYTNYNQK
FKGRVTMTADKSTSTAYMELSSLRSEDTAVYYCARYYDDHYSLDYWGQGTLVTVSSGGGGSGG
GGSGGGGSGGGGSEIVLTQSPATLSLSPGERVTLSCRASSSVSYLNWYQQKSGTSPKRWIYDA
SKVATGIPYRFSGSGSGTDFTLTISSLQAEDEATYYCQQRSNWPLTFGQGTKLEIK
SEQ ID NO: 19 (AA, Vulcanodon-27 anti-CD3 scFv)
QVQLVQSGAEVKKPGASVKVSCKASGYTFTRYTMHWVRQAPGQGLEWMGYINPSKGYTNYNQK
FKGRVTMTADKSTSTVYMELSSLRSEDTAVYYCARYYDDHYSLDYWGQGTLVTVSSGGGGSGG
GGSGGGGSGGGGSEIVMTQSPAIMSASPGERVTLSCRASSSVSYMNWYQQKSGTSPKRWIYDT
SKVASGVPYRFSGSGSGTDFTLTISSLQAEDEATYYCQQWSSNPLTFGQGTKLEIK
SEQ ID NO: 20 (AA, Vulcanodon-28 anti-CD3 scFv)
QVQLVQSGAEVKKPGASVKVSCKASGYTFTSYYMHWVRQAPGQGLEWMGYINPSKGYTNYNQK
FKGRVTMTADKSTSTVYMELSSLRSEDTAVYYCARYYDDHYSLDYWGQGTLVTVSSGGGGSGG
GGSGGGGSGGGGSEIVMTQSPAIMSASPGERVTLSCRASSSVSYMNWYQQKSGTSPKRWIYDT
SKVASGVPYRFSGSGSGTDFTLTISSLQAEDEATYYCQQWSSNPLTFGQGTKLEIK
SEQ ID NO: 21 (AA, Vulcanodon-29 anti-CD3 scFv)
QVQLVQSGAEVKKPGASVKVSCKASGYTFTRYTMHWVKQRPGQGLEWIGYINPSKGYTNYNQK
FKGRVTMTADKSTSTAYMELSSLRSEDTAVYYCARYYDDHYSLDYWGQGTLVTVSSGGGGSGG
GGSGGGGSGGGGSEIVLTQSPATLSLSPGERVTLSCRASSSVSYLNWYQQKSGTSPKRWIYDT
SKVATGIPYRFSGSGSGTDFTLTISSLQAEDEATYYCQQWSSNPLTFGQGTKLEIK
SEQ ID NO: 22 (AA, Vulcanodon-30 anti-CD3 scFv)
QVQLVQSGAEVKKPGASVKVSCKASGYTFTRYTMHWVKQRPGQGLEWIGYINPSKGYTNYNQK
FKGRVTMTADKSTSTAYMELSSLRSEDTAVYYCARYYDDHYSLDYWGQGTLVTVSSGGGGSGG
GGSGGGGSGGGGSEIVLTQSPATLSLSPGERVTLSCRASSSVSYLNWYQQKSGTSPKRWIYDA
SKVATGIPYRFSGSGSGTDFTLTISSLQAEDEATYYCQQRSNWPLTFGQGTKLEIK
SEQ ID NO: 23 (AA, Vulcanodon-31 anti-CD3 scFv)
QVQLVQSGAEVKKPGASVKVSCKASGYTFTSYYMHWVRQAPGQGLEWMGYINPSKGYTNYNQK
FKGRVTMTADKSTSTVYMELSSLRSEDTAVYYCARYYDDHYSLDYWGQGTLVTVSSGGGGSGG
GGSGGGGSGGGGSEIVLTQSPATLSLSPGERVTLSCRASSSVSYLNWYQQKSGTSPKRWIYDA
SKVATGIPYRFSGSGSGTDFTLTISSLQAEDEATYYCQQRSNWPLTFGQGTKLEIK
SEQ ID NO: 24 (AA, Vulcanodon-32 anti-CD3 scFv)
QVQLVQSGAEVKKLGASVKVSCKASGYTFTRYTMHWVKQAPGQGLEWMGYINPSRGYTNYNQK
FQGRVTMTRDTSISTAYMELSRLRSDDTAVYYCARYYDDHYSLDYWGQGTLVTVSSGGGGSGG
GGSGGGGSGGGGSDIQMTQSPSSLSASVGDRVTITCRASSSISYLNWYQQKSGTSPKRWIYDT
SKVASGVPYRFSGSGSGTDFTLTISSLQPEDFATYYCQQWSSNPLTFGAGTKLEIK
SEQ ID NO: 25 (AA, Vulcanodon-33 anti-CD3 scFv)
QVQLVQSGAEVKKPGASVKVSCKASGYTFTRYTMHWVRQRPGQGLEWMGYINPSRGYTNYNQK
FQGRVTMTRTTSSSTAYMELSSLRSDDTAVYYCARYYDDHYSLDYWGQGTLVTVSSGGGGSGG
GGSGGGGSGGGGSDIQMTQSPSSLSASVGDRVTITCRASSSVSYMNWYQQKSGTSPKRWIYDT
SKVASGVPYRFSGSGSGTDFTLTISSLQPEDFATYYCQQWSSNPLTFGAGTKLEIK
SEQ ID NO: 26 (AA, Vulcanodon-34 anti-CD3 scFv)
QVQLVQSGAEVKKLGASVKVSCKASGYTFTRYTMHWVKQAPGQGLEWIGYINPSRGYTNYNQK
FQGRVTMTRDTSISTAYMELSRLRSDDTAVYYCARYYDDHYSLDYWGQGTLVTVSSGGGGSGG
GGSGGGGSGGGGSDIQMTQSPSSLSASVGDRVTITCRASSSVSYMNWYQQKSGTSPKRWIYDT
SKVASGVPYRFSGSGSGTDFTLTISSLQAEDAATYYCQQWSSNPLTFGAGTKLEIK
SEQ ID NO: 27 (AA, Vulcanodon-35 anti-CD3 scFv)
QVQLVQSGAEVKKPGASVKVSCKASGYTFTRYTMHWVRQRPGQGLEWMGYINPSRGYTNYNQK
FQGRVTMTRTTSSSTAYMELSSLRSDDTAVYYCARYYDDHYSLDYWGQGTLVTVSSGGGGSGG
GGSGGGGSGGGGSDIQMTQSPSSLSASVGDRVTITCRASSSVSYMNWYQQKSGTSPKRWIYDT
SKVASGVPYRFSGSGSGTDFTLTISSLQAEDAATYYCQQWSSNPLTFGAGTKLEIK
SEQ ID NO: 28 (AA, Vulcanodon-56 anti-CD3 scFv)
QVQLVQSGAEVKKPGASVKVSCKASGYTFTRYTMHWVRQAPGQGLEWMGYINPSKGYTNYNQK
FKGRVTMTADKSTSTAYMELSSLRSEDTAVYYCARYYDDHYSLDYWGQGTLVTVSSGGGGSGG
GGSGGGGSGGGGSEIVMTQSPATLSVSPGERATLSCRASSSVSYLNWYQQKSGTSPKRWIYDT
SKVASGVPYRFSGSGSGTDFTLTISSLQAEDEATYYCQQWSSNPLTFGQGTKLEIK
SEQ ID NO: 29 (AA, Vulcanodon-57 humanized anti-CD3 scFv)
QVQLVQSGAEVKKPGASVKVSCKASGYTFTRYTMHWVRQAPGQGLEWMGYINPSKGYTNYNQK
FKGRVTITADKSTSTAYMELSSLRSEDTAVYYCARYYDDHYSLDYWGQGTLVTVSSGGGGSGG
GGSGGGGSGGGGSEIVMTQSPATLSVSPGERATLSCRASSSVSYLNWYQQKSGTSPKRWIYDT
SKVASGVPYRFSGSGSGTDFTLTISSLQAEDEATYYCQQWSSNPLTFGQGTKLEIK
SEQ ID NO: 30 (AA, Vulcanodon-58 humanized anti-CD3 scFv)
QVQLVQSGAEVKKPGASVKVSCKASGYTFTRYTMHWVRQAPGQGLEWMGYINPSKGYTNYNQK
FKGRVTMTADKSTSTAYMELSSLRSEDTAVYYCARYYDDHYSLDYWGQGTLVTVSSGGGGSGG
GGSGGGGSGGGGSEIVMTQSPATLSVSPGERATLSCRASSSVSYLNWYQQKPGTAPKRWIYDT
SKVASGVPYRFSGSGSGTDFTLTISSLQAEDEATYYCQQWSSNPLTFGQGTKLEIK
SEQ ID NO: 31 (AA, Vulcanodon-59 humanized anti-CD3 scFv)
QVQLVQSGAEVKKPGASVKVSCKASGYTFTRYTMHWVRQAPGQGLEWMGYINPSKGYTNYNQK
FKGRVTMTADKSTSTAYMELSSLRSEDTAVYYCARYYDDHYSLDYWGQGTLVTVSSGGGGSGG
GGSGGGGSGGGGSEIVMTQSPATLSVSPGERATLSCRASSSVSYLNWYQQKSGTSPKRWIYDT
SKLESGVPYRFSGSGSGTDFTLTISSLQSEDEATYYCQQWSSNPLTFGQGTKLEIK
SEQ ID NO: 32 (AA, Vulcanodon-60 humanized anti-CD3 scFv)
QVQLVQSGAEVKKPGASVKVSCKASGYTFTRYTMHWVRQAPGQGLEWMGYINPSKGYTNYNQK
FKGRVTMTADKSTSTAYMELSSLRSEDTAVYYCARYYDDHYSLDYWGQGTLVTVSSGGGGSGG
GGSGGGGSGGGGSEIVMTQSPATLSVSPGERATLSCRASSSVSYLNWYQQKPGSAPKRLIYDT
SKLESGVPYRFSGSGSGTDFTLTISSLQSEDEADYYCQQWSSNPLTFGQGTKLEIK
SEQ ID NO: 33 (AA, Vulcanodon-61 humanized anti-CD3 scFv)
QVQLVQSGAEVKKPGASVKVSCKASGYTFTRYTMHWVRQAPGQGLEWMGYINPSKGYTNYNQK
FKGRVTITADKSTSTAYMELSSLRSEDTAVYYCARYYDDHYSLDYWGQGTLVTVSSGGGGSGG
GGSGGGGSGGGGSEIVMTQSPATLSVSPGERATLSCRASSSVSYLNWYQQKPGTAPKRWIYDT
SKVASGVPYRFSGSGSGTDFTLTISSLQAEDEATYYCQQWSSNPLTFGQGTKLEIK
SEQ ID NO: 34 (AA, Vulcanodon-62 humanized anti-CD3 scFv)
QVQLVQSGAEVKKPGASVKVSCKASGYTFTRYTMHWVRQAPGQGLEWMGYINPSKGYTNYNQK
FKGRVTITADKSTSTAYMELSSLRSEDTAVYYCARYYDDHYSLDYWGQGTLVTVSSGGGGSGG
GGSGGGGSGGGGSEIVMTQSPATLSVSPGERATLSCRASSSVSYLNWYQQKSGTSPKRWIYDT
SKLESGVPYRFSGSGSGTDFTLTISSLQSEDEATYYCQQWSSNPLTFGQGTKLEIK
SEQ ID NO: 35 (AA, Vulcanodon-63 humanized anti-CD3 scFv)
QVQLVQSGAEVKKPGASVKVSCKASGYTFTRYTMHWVRQAPGQGLEWMGYINPSKGYTNYNQK
FKGRVTITADKSTSTAYMELSSLRSEDTAVYYCARYYDDHYSLDYWGQGTLVTVSSGGGGSGG
GGSGGGGSGGGGSEIVMTQSPATLSVSPGERATLSCRASSSVSYLNWYQQKPGTAPKRWIYDT
SKLASGVPYRFSGSGSGTDFTLTISSLQSEDEATYYCQQWSSNPLTFGQGTKLEIK
SEQ ID NO: 36 (AA, Vulcanodon-64 humanized anti-CD3 scFv)
QVQLVQSGAEVKKPGASVKVSCKASGYTFTRYTMHWVRQAPGQGLEWMGYINPSKGYTNYNQK
FKGRVTITADKSTSTAYMELSSLRSEDTAVYYCARYYDDHYSLDYWGQGTLVTVSSGGGGSGG
GGSGGGGSGGGGSEIVMTQSPATLSVSPGERATLSCRASSSVSYLNWYQQKPGSAPKRWIYDT
SKLESGVPYRFSGSGSGTDFTLTISSLQSEDEATYYCQQWSSNPLTFGQGTKLEIK
SEQ ID NO: 37 (AA, Vulcanodon-65 humanized anti-CD3 scFv)
QVQLVQSGAEVKKPGASVKVSCKASGYTFTRYTMHWVRQAPGQGLEWMGYINPSKGYTNYNQK
FKGRVTITADKSTSTAYMELSSLRSEDTAVYYCARYYDDHYSLDYWGQGTLVTVSSGGGGSGG
GGSGGGGSGGGGSEIVMTQSPATLSVSPGERATLSCRASSSVSYLNWYQQKPGSAPKRLIYDT
SKLESGVPYRFSGSGSGTDFTLTISSLQSEDEADYYCQQWSSNPLTFGQGTKLEIK
SEQ ID NO: 38 (AA, Vulcanodon-66 humanized anti-CD3 scFv)
QVQLVQSGAEVKKPGASVKVSCKASGYTFTRYTMHWVRQAPGQGLEWMGYINPSKGYTNYNQK
FKGRVTITADKSTSTAYMELSSLRSEDTAVYYCARYYDDHYSLDYWGQGTLVTVSSGGGGSGG
GGSGGGGSGGGGSEIVMTQSPATLSVSPGERATLSCRASSSVSYLNWYQQKSGSAPKRWIYDT
SKLESGVPYRFSGSGSGTDFTLTISSLQAEDEADYYCQQWSSNPLTFGQGTKLEIK
SEQ ID NO: 39 (AA, S′ anti-BCMA scFv)
DIVLTQSPASLAVSPGQRATITCRASESVTILGSHLIHWYQQKPGQPPKLLINLASNVNTGVPA
RFSGSGSGTDFTLTISSVEPEDTANYYCLQSRTLPRTFGQGTKVEIKGSTSGSGKPGSGEGSTK
GQIQLVQSGPELKKPGGSVKISCKASGYTFTDYSINWVRQAPGKGLEWVGWINTETREPAYAYD
FTGRFTFSADTSKSMAYLQINSLRAEDTAVYYCALDYTYGMDYWGQGTLVTVSS
SEQ ID NO: 40 (AA, R1 anti-BCMA scFv)
DIVLTQSPPSLAMSLGKRATISCRASESVTILGSHLIHWYQQKPGQPPTLLIQLASNVQTGVPA
RFSGSGSRTDFTLTIDPVEEDDVAVYYCLQSRTIPRTFGGGTKLEIKGSTSGSGKPGSGEGSTK
GQIQLVQSGPELKKPGETVKISCKASGYTFTDYSINWVKRAPGKGLKWMGWINTETREPAYAYD
FRGRFAFSLETSASTAYLQINNLKYEDTATYFCALDYSYAMDYWGQGTSVTVSS
SEQ ID NO: 41 (AA, R′MQD scFv)
DIVLTQSPASLAVSPGQRATITCRASESVSFLGINLIHWYQQKPGQPPKLLIYSASNLQSGVPA
RFSGSGSGTDFTLTISSVEPEDTANYYCLQSRTLPRTFGQGTKVEIKGSTSGSGKPGSGEGSTK
GQIQLVQSGPELKKPGGSVKISCKASGYTFTSYSINWVRQAPGKGLEWVGWINTETREPAYAQG
FTGRFTFSADTSKSMAYLQINSLRAEDTAVYYCALDYLYSLDFWGQGTLVTVSS
SEQ ID NO: 42 (AA, J1 anti-BCMA scFv)
DIVMTQSPDSLSVSLGERATINCRASESVTILGSHLIHWYQQKPGQPPKLLIQLASNVQTGVPD
RFSGSGSGTDFTLTISSLQAEDVAVYYCLQSRTIPRTFGGGTKVEIKGSTSGSGKPGSGEGSTK
GQIQLVQSGAEVKKPGASVKISCKASGYTFTDYSINWVRQAPGQGLEWMGWINTETREPAYAYD
FRGRVTMTRDTSASTAYLQISSLKAEDTAVYFCALDYSYAMDYWGQGSLVTVSS
SEQ ID NO: 43 (AA, 17′ anti-BCMA scFv)
DIVLTQSPASLAVSPGQRATITCRASESVTILGSHLIHWYQQKPGQPPKLLIQLASNVQTGVPA
RFSGSGSGTDFTLTISSVEPEDTANYYCLQSRTIPRTFGQGTKVEIKGSTSGSGKPGSGEGSTK
GQIQLVQSGPELKKPGGSVKISCKASGYTFTDYSINWVRQAPGKGLEWVGWINTETREPAYAYD
FTGRFTFSADTSKSMAYLQINSLRAEDTAVYYCALDYSYAMDYWGQGTLVTVSS
SEQ ID NO: 44 (AA, J3 anti-BCMA scFv)
DIVLTQSPASLAVSLGKRATISCRASESVTILGSHLIHWYQQKPGQPPKLLIYLASNVQTGVPA
RFSGSGSGTDFTLTISSLEAEDVAVYYCLQSRTIPRTFGQGTKLEIKGSTSGSGKPGSGEGSTK
GQVQLVQSGAEVKKPGASVKISCKASGYTFTDYSINWVRQAPGQGLEWMGWINTETREPAYAYD
FRGRVTMTRDTSASTAYLQISSLKAEDTAVYFCALDYSYAMDYWGQGSLVTVSS
SEQ ID NO: 45 (AA, J4 anti-BCMA scFv)
DIVMTQSPDSLSVSLGERATINCRASESVTILGSHLIHWYQQKPGQPPKLLIQLASNVQTGVPD
RFSGSGSGTDFTLTISSLQAEDVAVYYCLQSRTIPRTFGGGTKVEIKGSTSGSGKPGSGEGSTK
GQIQLVQSGAEVKKPGESVKISCKASGYTFTDYSINWVRQAPGQGLKWMGWINTETREPAYAYD
FRGRFAFSLDTSASTAYLQISSLKAEDTAVYFCALDYSYAMDYWGQGTLVTVSS
SEQ ID NO: 46 (AA, J5 anti-BCMA scFv)
DIVLTQSPASLAVSLGERATISCRASESVSILGSHYLAWYQQKPGQPPKLLIYLASNLQTGVPA
RFSGSGSGTDFTLTISSLEAEDVAVYYCQQSRTIPRTFGQGTKLEIKGSTSGSGKPGSGEGSTK
GQIQLVQSGAEVKKPGESVKISCKASGYTFTDYSINWVRQAPGQGLKWMGWINTETREPAYAYD
FRGRFAFSLDTSASTAYLQISSLKAEDTAVYFCALDYSYAMDYWGQGTLVTVSS
SEQ ID NO: 47 (AA, Yi1 anti-BCMA scFv)
DIVLTQSPSSLSASVGDRATISCRASESVTILGSHLIHWYQQKPGQAPKLLIQLASNVQTGVPA
RFSGSGSGTDFTLTISSVEPDDVAVYYCLQSRTIPRTFGGGTKLEIKGSTSGSGKPGSGEGSTK
GQIQLVQSGPELKKPGGSVKISCAASGYTFTDYSINWVKQAPGKGLEWVGWINTETREPAYAYD
FRGRFTFSLDTSKSTAYLQMNSLRAEDTAVYYCALDYSYAMDYWGQGTLVTVSS
SEQ ID NO: 48 (AA, CAS2 anti-BCMA scFv)
DIVLTQSPASLAVSPGQRATISCRASESVTILGSHLIHWYQQKPGQPPKLLIQLASNVQTGVPA
RFSGSGSRTDFTLTISSLEPEDVAVYYCLQSRTIPRTFGQGTKVEIKGSTSGSGKPGSGEGSTK
GQITLKESGPTLVKPTQTLTLSCKASGYTFTDYSINWVRRAPGKGLEWMGWINTETREPAYAYD
FRGRFVFSLDTSVSMAYLQISSLKAEDTAVYYCALDYSYAMDYWGQGTLVTVSS
SEQ ID NO: 49 (AA, Yi31 anti-BCMA scFv)
DIVLTQSPSSLSASVGDRATISCRASESVTILGSHLIHWYQQKPGQAPKLLIQLASNVQTGVPA
RFSGSGSGTDFTLTISSVEPEDVAVYYCLQSRTIPRTFGQGTKVEIKGSTSGSGKPGSGEGSTK
GQIQLVQSGPELKKPGGSVKISCAASGYTFTDYSINWVKQAPGKGLEWVGWINTETREPAYAYD
FRGRFTFSADTSKSTAYLQMNSLRAEDTAVYYCALDYSYAMDYWGQGTLVTVSS
SEQ ID NO: 50 (AA, Yi32 anti-BCMA scFv)
DIVLTQSPSSLSASVGDRATISCRASESVTILGSHLIHWYQQKPGQAPKLLIQLASNVQTGVPA
RFSGSGSGTDFTLTISSVEPEDVAVYYCLQSRTIPRTFGQGTKVEIKGSTSGSGKPGSGEGSTK
GQIQLVQSGPELKKPGGSVKISCAASGYTFTDYSINWVRQAPGKGLEWVGWINTETREPAYAYD
FRGRFTFSADTSKSTAYLQMNSLRAEDTAVYYCALDYSYAMDYWGQGTLVTVSS
SEQ ID NO: 51 (AA, Yi33 anti-BCMA scFv)
DIVLTQSPSSLSASVGDRATISCRASESVTILGSHLIHWYQQKPGQAPKLLIQLASNVQTGVPA
RFSGSGSGTDFTLTISSVEPEDVAVYYCLQSRTIPRTFGQGTKVEIKGSTSGSGKPGSGEGSTK
GQIQLVESGAEVKKPGGSVKISCAASGYTFTDYSINWVKQAPGKGLEWVGWINTETREPAYAYD
FRGRFTFSLDTSKSTAYLQMNSLRAEDTAVYYCALDYSYAMDYWGQGTLVTVSS
SEQ ID NO: 52 (AA, Yi34 anti-BCMA scFv)
DIVLTQSPSSLSASVGDRATISCRASESVTILGSHLIHWYQQKPGQAPKLLIQLASNVQTGVPA
RFSGSGSGTDFTLTISSVEPEDVAVYYCLQSRTIPRTFGQGTKVEIKGSTSGSGKPGSGEGSTK
GQIQLVESGAEVKKPGGSVKVSCKASGYTFTDYSINWVRQAPGKGLEWVGWINTETREPAYAYD
FRGRFAISAETSKSTAYLQMNSLRAEDTAVYYCALDYSYAMDYWGQGTLVTVSS
SEQ ID NO: 53 (AA, Yi37 anti-BCMA scFv)
DIVLTQSPASLAVSPGQRATITCRASESVTILGSHLIHWYQQKPGQPPKLLIQLASNVQTGVPA
RFSGSGSGTDFTLTISSVEPEDTANYYCLQSRTIPRTFGQGTKVEIKGSTSGSGKPGSGEGSTK
GQIQLVQSGPELKKPGGSVKISCKASGYTFTDYSINWVRQAPGKGLEWVGWINTETREPAYAYD
FTGRFTFSADTSKSMAYLQINSLRAEDTAVYYCALDYSYAMDYWGQGTLVTVSS
SEQ ID NO: 54 (AA, 1′ anti-BCMA scFv)
DIVLTQSPSSLSASVGDRATISCRASESVTILGSHLIHWYQQKPGQAPKLLIQLASNVQTGVPA
RFSGSGSGTDFTLTISSVEPEDVAVYYCLQSRTIPRTFGQGTKVEIKGSTSGSGKPGSGEGSTK
GQIQLVQSGPELKKPGGSVKISCAASGYTFTSYDINWVRQAPGKGLEWVGWINTETREPAYAYD
FRGRFTFSADTSKSTAYLQMNSLRAEDTAVYYCALDYSYAMDYWGQGTLVTVSS
SEQ ID NO: 55 (AA, 4′ anti-BCMA scFv)
DIVLTQSPSSLSASVGDRATISCRASESVTILGSHLIHWYQQKPGQAPKLLIQLASNVQTGVPA
RFSGSGSGTDFTLTISSVEPEDVAVYYCLQSRTIPRTFGQGTKVEIKGSTSGSGKPGSGEGSTK
GQIQLVQSGPELKKPGGSVKISCAASGYTFTDYSINWVRQAPGKGLEWVGWINTETREPAYAYD
FRGRFTFSADTSKSTAYLQMNSLRAEDTAVYYCALDYTYGMDYWGQGTLVTVSS
SEQ ID NO: 56 (AA, 8′ anti-BCMA scFv)
DIVLTQSPSSLSASVGDRATISCRASESVSFLGINLIHWYQQKPGQAPKLLIYSASNLQSGVPA
RFSGSGSGTDFTLTISSVEPEDVAVYYCLQSRTLPRTFGQGTKVEIKGSTSGSGKPGSGEGSTK
GQIQLVQSGPELKKPGGSVKISCAASGYTFTDYSINWVRQAPGKGLEWVGWINTETREPAYAYD
FRGRFTFSADTSKSTAYLQMNSLRAEDTAVYYCALDYSYAMDYWGQGTLVTVSS
SEQ ID NO: 57 (AA, 9′ anti-BCMA scFv)
DIVLTQSPSSLSASVGDRATISCRASESVSFLGINLIHWYQQKPGQAPKLLIYSASNLQSGVPA
RFSGSGSGTDFTLTISSVEPEDVAVYYCLQSKNFPRTFGQGTKVEIKGSTSGSGKPGSGEGSTK
GQIQLVQSGPELKKPGGSVKISCAASGYTFTDYSINWVRQAPGKGLEWVGWINTETREPAYAYD
FRGRFTFSADTSKSTAYLQMNSLRAEDTAVYYCALDYSYAMDYWGQGTLVTVSS
SEQ ID NO: 58 (AA, 10′ anti-BCMA scFv)
DIVLTQSPSSLSASVGDRATISCRASESVTILGSHLIHWYQQKPGQAPKLLIQLASNVQTGVPA
RFSGSGSGTDFTLTISSVEPEDVAVYYCLQSRTIPRTFGQGTKVEIKGSTSGSGKPGSGEGSTK
GQIQLVQSGPELKKPGGSVKISCAASGYTFTSYSINWVRQAPGKGLEWVGWINTETREPAYAQG
FTGRFTFSADTSKSTAYLQMNSLRAEDTAVYYCALDYLYSLDFWGQGTLVTVSS
SEQ ID NO: 59 (AA, 11′ anti-BCMA scFv)
DIVLTQSPSSLSASVGDRATISCRASESVTILGSHLIHWYQQKPGQAPKLLIQLASNVQTGVPA
RFSGSGSGTDFTLTISSVEPEDVAVYYCLQSRTIPRTFGQGTKVEIKGSTSGSGKPGSGEGSTK
GQIQLVQSGPELKKPGGSVKISCAASGYTFTSYDINWVRQAPGKGLEWVGWINTNTGNPTYAQG
FTGRFTFSADTSKSTAYLQMNSLRAEDTAVYYCALDYTYGMDYWGQGTLVTVSS
SEQ ID NO: 60 (AA, 12′ anti-BCMA scFv)
DIVLTQSPSSLSASVGDRATISCRASESVTILGSHLIHWYQQKPGQAPKLLIQLASNVQTGVPA
RFSGSGSGTDFTLTISSVEPEDVAVYYCLQSKNFPRTFGQGTKVEIKGSTSGSGKPGSGEGSTK
GQIQLVQSGPELKKPGGSVKISCAASGYTFTSYDINWVRQAPGKGLEWVGWINTETREPAYAYD
FRGRFTFSADTSKSTAYLQMNSLRAEDTAVYYCALDYLYSLDFWGQGTLVTVSS
SEQ ID NO: 61 (AA, 13′ anti-BCMA scFv)
DIVLTQSPSSLSASVGDRATISCRASESVSFLGINLIHWYQQKPGQAPKLLIYLASNLETGVPA
RFSGSGSGTDFTLTISSVEPEDVAVYYCLQSRTLPRTFGQGTKVEIKGSTSGSGKPGSGEGSTK
GQIQLVQSGPELKKPGGSVKISCAASGYTFTSYSINWVRQAPGKGLEWVGWINTETREPAYAQG
FTGRFTFSADTSKSTAYLQMNSLRAEDTAVYYCALDYLYSLDFWGQGTLVTVSS
SEQ ID NO: 62 (AA, 21′ anti-BCMA scFv)
DIVLTQSPSSLSASVGDRATISCRASESVTILGSHLIHWYQQKPGQAPKLLINLASNVNTGVPA
RFSGSGSGTDFTLTISSVEPEDVAVYYCLQSRTIPRTFGQGTKVEIKGSTSGSGKPGSGEGSTK
GQIQLVQSGPELKKPGGSVKISCAASGYTFTDYSINWVRQAPGKGLEWVGWINTETREPAYAYD
FRGRFTFSADTSKSTAYLQMNSLRAEDTAVYYCALDYSYAMDYWGQGTLVTVSS
SEQ ID NO: 63 (AA, 2′ anti-BCMA scFv)
DIVLTQSPSSLSASVGDRATISCRASESVTILGSHLIHWYQQKPGQAPKLLIQLASNVQTGVPA
RFSGSGSGTDFTLTISSVEPEDVAVYYCLQSRTLPRTFGQGTKVEIKGSTSGSGKPGSGEGSTK
GQIQLVQSGPELKKPGGSVKISCAASGYTFTDYSINWVRQAPGKGLEWVGWINTETREPAYAYD
FRGRFTFSADTSKSTAYLQMNSLRAEDTAVYYCALDYSYAMDYWGQGTLVTVSS
SEQ ID NO: 64 (AA, 3′ anti-BCMA scFv)
DIVLTQSPSSLSASVGDRATISCRASESVTILGSHLIHWYQQKPGQAPKLLIQLASNVQTGVPA
RFSGSGSGTDFTLTISSVEPEDVAVYYCLQSKNFPRTFGQGTKVEIKGSTSGSGKPGSGEGSTK
GQIQLVQSGPELKKPGGSVKISCAASGYTFTDYSINWVRQAPGKGLEWYGWINTETREPAYAYD
FRGRFTFSADTSKSTAYLQMNSLRAEDTAVYYCALDYSYAMDYWGQGTLVTVSS
SEQ ID NO: 65 (AA, linker 1)
GGGGSGGGGSGGGGS
SEQ ID NO: 66 (AA, linker 2)
GGGGS
SEQ ID NO: 67 (AA, inker 3)
VEGGSGGSGGSGGSGGVD
SEQ ID NO: 68 (AA, linker 4)
GGGGSGGGGSGGGGSGGGGS
Sequence A (AA, linker 5)
AAS
SEQ ID NO: 70 (AA, linker 6)
AAAL
SEQ ID NO: 71 (AA, linker R′)
GSTSGSGKPGSGEGSTKG
SEQ ID NO: 72 (AA, linker 8)
SSGGSGGSGGSGGSGGSGGSGGSGGSGGSGS
SEQ ID NO: 73 (AA, linker 9)
GSGGSGGSGGSGGSGGSGGSGGSGGSGGSGS
SEQ ID NO: 74 (AA, human serum albumin)
KWVTFISLLFLFSSAYSRGVFRRDAHKSEVAHRFKDLGEENFKALVLIAFAQYLQQCPFEDHVK
LVNEVTEFAKTCVADESAENCDKSLHTLFGDKLCTVATLRETYGEMADCCAKQEPERNECFLQH
KDDNPNLPRLVRPEVDVMCTAFHDNEETFLKKYLYEIARRHPYFYAPELLFFAKRYKAAFTECC
QAADKAACLLPKLDELRDEGKASSAKQRLKCASLQKFGERAFKAWAVARLSQRFPKAEFAEVSK
LVTDLTKVHTECCHGDLLECADDRADLAKYICENQDSISSKLKECCEKPLLEKSHCIAEVENDE
MPADLPSLAADFVESKDVCKNYAEAKDVFLGMFLYEYARRHPDYSVVLLLRLAKTYKTTLEKCC
AAADPHECYAKVFDEFKPLVEEPQNLIKQNCELFEQLGEYKFQNALLVRYTKKVPQVSTPTLVE
VSRNLGKVGSKCCKHPEAKRMPCAEDYLSVVLNQLCVLHEKTPVSDRVTKCCTESLVNRRPCFS
ALEVDETYVPKEFNAETFTFHADICTLSEKERQIKKQTALVELVKHKPKATKEQLKAVMDDFAA
FVEKCCKADDKETCFAEEGKKLVAASRAALGL
SEQ ID NO: 75 (AA, Diplodocus-1)
MGWSCIILFLVATATGVHSDYKDDDDKDIQLTQSPASLAVSLGQRATISCKASQSVDYDGDSYL
NWYQQIPGQPPKLLIYDASNLVSGIPPRFSGSGSGTDFTLNIHPVEKVDAATYHCQQSTEDPWT
FGGGTKLEIKGGGGSGGGGSGGGGSQVQLQQSGAELVRPGSSVKISCKASGYAFSSYWMNWVKQ
RPGQGLEWIGQIWPGDGDTNYNGKFKGKATLTADESSSTAYMQLSSLASEDSAVYFCARRETTT
VGRYYYAMDYWGQGTTVTVSSGGGGSDIKLQQSGAELARPGASVKMSCKTSGYTFTRYTMHWVK
QRPGQGLEWIGYINPSRGYTNYNQKFKDKATLTTDKSSSTAYMQLSSLTSEDSAVYYCARYYDD
HYCLDYWGQGTTLTVSSVEGGSGGSGGSGGSGGVDDIQLTQSPAIMSASPGEKVTMTCRASSSV
SYMNWYQQKSGTSPKRWIYDTSKVASGVPYRFSGSGSGTSYSLTISSMEAEDAATYYCQQWSSN
PLTFGAGTKLELKHHHHHH
SEQ ID NO: 76 (AA, Diplodocus-2)
MGWSCIILFLVATATGVHSDIVLTQSPASLAVSPGQRATITCRASESVSFLGINLIHWYQQKPG
QPPKLLIYSASNLQSGVPARFSGSGSGTDFTLTISSVEPEDTANYYCLQSRTLPRTFGQGTKVE
IKGSTSGSGKPGSGEGSTKGQIQLVQSGPELKKPGGSVKISCKASGYTFTSYSINWVRQAPGKG
LEWVGWINTETREPAYAQGFTGRFTFSADTSKSMAYLQINSLRAEDTAVYYCALDYLYSLDFWG
QGTLVTVSSGGGGSDIKLQQSGAELARPGASVKMSCKTSGYTFTRYTMHWVKQRPGQGLEWIGY
INPSRGYTNYNQKFKDKATLTTDKSSSTAYMQLSSLTSEDSAVYYCARYYDDHYCLDYWGQGTT
LTVSSVEGGSGGSGGSGGSGGVDDIQLTQSPAIMSASPGEKVTMTCRASSSVSYMNWYQQKSGT
SPKRWIYDTSKVASGVPYRFSGSGSGTSYSLTISSMEAEDAATYYCQQWSSNPLTFGAGTKLEL
KHHHHHH
SEQ ID NO: 77 (AA, Diplodocus-3)
MGWSCIILFLVATATGVHSDIVLTQSPASLAVSPGQRATITCRASESVSFLGINLIHWYQQKPG
QPPKLLIYSASNLQSGVPARFSGSGSGTDFTLTISSVEPEDTANYYCLQSRTLPRTFGQGTKVE
IKGSTSGSGKPGSGEGSTKGQIQLVQSGPELKKPGGSVKISCKASGYTFTSYSINWVRQAPGKG
LEWVGWINTETREPAYAQGFTGRFTFSADTSKSMAYLQINSLRAEDTAVYYCALDYLYSLDFWG
QGTLVTVSSGGGGSQVQLVESGGGVVQPGRSLRLSCSSSGYTETRYTMHWVRQAPGKGLEWIGI
NPSRGYTNYNQKVKDRFTISTDKSKSTAFLQMDSLRPEDTAVYYCARYYDDHYCLDYWGQGTPV
TVSSGGGGSGGGGSGGGGSGGGGSDIQMTQSPSSLSASVGDRVTITCSASSSVSYMNWYQQTPG
KAPKRWIYDTSKLASGVPSRFSGSGSGTDYTFTISSLQPEDIATYYCQQWSSNPFTFGQGTKLQ
ITRHHHHHH
SEQ ID NO: 78 (AA, Diplodocus-4)
MGWSCIILFLVATATGVHSDIVLTQSPASLAVSPGQRATITCRASESVSFLGINLIHWYQQKPG
QPPKLLIYSASNLQSGVPARFSGSGSGTDFTLTISSVEPEDTANYYCLQSRTLPRTFGQGTKVE
IKGSTSGSGKPGSGEGSTKGQIQLVQSGPELKKPGGSVKISCKASGYTFTSYSINWVRQAPGKG
LEWVGWINTETREPAYAQGFTGRFTFSADTSKSMAYLQINSLRAEDTAVYYCALDYLYSLDFWG
QGTLVTVSSGGGGSQVQLVQSGAEVKKPGSSVKVSCKASGYKFTSYVMHWVRQAPGQGLEWMGY
INPYNDVTKYNEKFKGRATITADESTNTAYMELSSLRSEDTAFYFCARGSYYDYDGFVYWGQGT
LVTVSSGGGGSGGGGSGGGGSGGGGSDIQMTQSPSTLSASVGDRVTITCSATSSVSYMHWYQQK
PGKAPKRLMYDTSKLASGVPARFIGSGSGTEFTLTISSLQPDDFATYYCQQWSSNPLTFGGGTK
VEIKHHHHHH
SEQ ID NO: 79 (AA, Diplodocus-5)
MGWSCIILFLVATATGVHSDIVLTQSPASLAVSPGQRATITCRASESVSFLGINLIHWYQQKPG
QPPKLLIYSASNLQSGVPARFSGSGSGTDFTLTISSVEPEDTANYYCLQSRTLPRTFGQGTKVE
IKGSTSGSGKPGSGEGSTKGQIQLVQSGPELKKPGGSVKISCKASGYTFTSYSINWVRQAPGKG
LEWVGWINTETREPAYAQGFTGRFTFSADTSKSMAYLQINSLRAEDTAVYYCALDYLYSLDFWG
QGTLVTVSSGGGGSQVQLVQSGAEVKKPGSSVKVSCKASGYKFTSYVMHWVKQAPGQGLEWIGY
INPYNDVTKYNEKFKGKATLTADESTNTAYMELSSLRSEDTAVHYCARGSYYDYDGFVYWGQGT
LVTVSSGGGGSGGGGSGGGGSGGGGSDIQMTQSPSTLSASVGDRVTMTCSATSSVSYMHWYQQK
PGKAPKRWIYDTSKLASGVPARFIGSGSGTEFTLTISSLQPDDFATYYCQQWSSNPLTFGGGTK
VEIKHHHHHH
SEQ ID NO: 80 (AA, Diplodocus-6)
MGWSCIILFLVATATGVHSDIVLTQSPASLAVSPGQRATITCRASESVSFLGINLIHWYQQKPG
QPPKLLIYSASNLQSGVPARFSGSGSGTDFTLTISSVEPEDTANYYCLQSRTLPRTFGQGTKVE
IKGSTSGSGKPGSGEGSTKGQIQLVQSGPELKKPGGSVKISCKASGYTFTSYSINWVRQAPGKG
LEWVGWINTETREPAYAQGFTGRFTFSADTSKSMAYLQINSLRAEDTAVYYCALDYLYSLDFWG
QGTLVTVSSGGGGSQVQLQQSGAELARPGASVKMSCKTSGYTFTRYTMHWVKQRPGQGLEWIGY
INPSRGYTNYNQKFKDKATLTTDKSSSTAYMQLSSLTSEDSAVYYCARYYDDHYSLDYWGQGTT
LTVSSGGGGSGGGGSGGGGSGGGGSQIVLTQSPAIMSASPGEKVTMTCRASSSVSYMNWYQQKS
GTSPKRWIYDTSKVASGVPYRFSGSGSGTSYSLTISSMEAEDAATYYCQQWSSNPLTFGAGTKL
ELKHHHHHH
SEQ ID NO: 81 (AA, Diplodocus-7)
MGWSCIILFLVATATGVHSDIVLTQSPASLAVSPGQRATITCRASESVSFLGINLIHWYQQKPG
QPPKLLIYSASNLQSGVPARFSGSGSGTDFTLTISSVEPEDTANYYCLQSRTLPRTFGQGTKVE
IKGSTSGSGKPGSGEGSTKGQIQLVQSGPELKKPGGSVKISCKASGYTFTSYSINWVRQAPGKG
LEWVGWINTETREPAYAQGFTGRFTFSADTSKSMAYLQINSLRAEDTAVYYCALDYLYSLDFWG
QGTLVTVSSAASKWVTFISLLFLFSSAYSRGVFRRDAHKSEVAHRFKDLGEENFKALVLIAFAQ
YLQQCPFEDHVKLVNEVTEFAKTCVADESAENCDKSLHTLFGDKLCTVATLRETYGEMADCCAK
QEPERNECFLQHKDDNPNLPRLVRPEVDVMCTAFHDNEETFLKKYLYEIARRHPYFYAPELLFF
AKRYKAAFTECCQAADKAACLLPKLDELRDEGKASSAKQRLKCASLQKFGERAFKAWAVARLSQ
RFPKAEFAEVSKLVTDLTKVHTECCHGDLLECADDRADLAKYICENQDSISSKLKECCEKPLLE
KSHCIAEVENDEMPADLPSLAADFVESKDVCKNYAEAKDVFLGMFLYEYARRHPDYSVVLLLRL
AKTYKTTLEKCCAAADPHECYAKVFDEFKPLVEEPQNLIKQNCELFEQLGEYKFQNALLVRYTK
KVPQVSTPTLVEVSRNLGKVGSKCCKHPEAKRMPCAEDYLSVVLNQLCVLHEKTPVSDRVTKCC
TESLVNRRPCFSALEVDETYVPKEFNAETFTFHADICTLSEKERQIKKQTALVELVKHKPKATK
EQLKAVMDDFAAFVEKCCKADDKETCFAEEGKKLVAASRAALGLAAALQVQLVESGGGVVQPGR
SLRLSCSSSGYTETRYTMHWVRQAPGKGLEWIGINPSRGYTNYNQKVKDRFTISTDKSKSTAFL
QMDSLRPEDTAVYYCARYYDDHYCLDYWGQGTPVTVSSGGGGSGGGGSGGGGSGGGGSDIQMTQ
SPSSLSASVGDRVTITCSASSSVSYMNWYQQTPGKAPKRWIYDTSKLASGVPSRFSGSGSGTDY
TFTISSLQPEDIATYYCQQWSSNPFTFGQGTKLQITRHHHHHH
SEQ ID NO: 82 (AA, Diplodocus-8)
MGWSCIILFLVATATGVHSDIVLTQSPASLAVSPGQRATITCRASESVSFLGINLIHWYQQKPG
QPPKLLIYSASNLQSGVPARFSGSGSGTDFTLTISSVEPEDTANYYCLQSRTLPRTFGQGTKVE
IKGSTSGSGKPGSGEGSTKGQIQLVQSGPELKKPGGSVKISCKASGYTFTSYSINWVRQAPGKG
LEWVGWINTETREPAYAQGFTGRFTFSADTSKSMAYLQINSLRAEDTAVYYCALDYLYSLDFWG
QGTLVTVSSGGGGSQVQLQQSGAELARPGASVKMSCKTSGYTFTRYTMHWVKQRPGQGLEWIGY
INPSRGYTNYNQKFKDKATLTTDKSSSTAYMQLSSLTSEDSAVYYCARYYDDHYSLDYWGQGTT
LTVSSGGGGSGGGGSGGGGSGGGGSQIVLTQSPAIMSASPGEKVTMTCRASSSVSYMNWYQQKS
GTSPKRWIYDTSKVASGVPYRFSGSGSGTSYSLTISSMEAEDAATYYCQQWSSNPLTFGAGTKL
ELKAASKWVTFISLLFLFSSAYSRGVFRRDAHKSEVAHRFKDLGEENFKALVLIAFAQYLQQCP
FEDHVKLVNEVTEFAKTCVADESAENCDKSLHTLFGDKLCTVATLRETYGEMADCCAKQEPERN
ECFLQHKDDNPNLPRLVRPEVDVMCTAFHDNEETFLKKYLYEIARRHPYFYAPELLFFAKRYKA
AFTECCQAADKAACLLPKLDELRDEGKASSAKQRLKCASLQKFGERAFKAWAVARLSQRFPKAE
FAEVSKLVTDLTKVHTECCHGDLLECADDRADLAKYICENQDSISSKLKECCEKPLLEKSHCIA
EVENDEMPADLPSLAADFVESKDVCKNYAEAKDVFLGMFLYEYARRHPDYSVVLLLRLAKTYKT
TLEKCCAAADPHECYAKVFDEFKPLVEEPQNLIKQNCELFEQLGEYKFQNALLVRYTKKVPQVS
TPTLVEVSRNLGKVGSKCCKHPEAKRMPCAEDYLSVVLNQLCVLHEKTPVSDRVTKCCTESLVN
RRPCFSALEVDETYVPKEFNAETFTFHADICTLSEKERQIKKQTALVELVKHKPKATKEQLKAV
MDDFAAFVEKCCKADDKETCFAEEGKKLVAASRAALGLHHHHHH
SEQ ID NO: 83 (AA, Diplodocus-9)
MGWSCIILFLVATATGVHSDIVLTQSPASLAVSPGQRATITCRASESVSFLGINLIHWYQQKPG
QPPKLLIYSASNLQSGVPARFSGSGSGTDFTLTISSVEPEDTANYYCLQSRTLPRTFGQGTKVE
IKGSTSGSGKPGSGEGSTKGQIQLVQSGPELKKPGGSVKISCKASGYTFTSYSINWVRQAPGKG
LEWVGWINTETREPAYAQGFTGRFTFSADTSKSMAYLQINSLRAEDTAVYYCALDYLYSLDFWG
QGTLVTVSSGGGGSQVQLVESGGGVVQPGRSLRLSCSSSGYTETRYTMHWVRQAPGKGLEWIGI
NPSRGYTNYNQKVKDRFTISTDKSKSTAFLQMDSLRPEDTAVYYCARYYDDHYCLDYWGQGTPV
TVSSVEGGSGGSGGSGGSGGVDDIQMTQSPSSLSASVGDRVTITCSASSSVSYMNWYQQTPGKA
PKRWIYDTSKLASGVPSRFSGSGSGTDYTFTISSLQPEDIATYYCQQWSSNPFTFGQGTKLQIT
RHHHHHH
SEQ ID NO: 84 (AA, Diplodocus-10)
MGWSCIILFLVATATGVHSDIVLTQSPASLAVSPGQRATITCRASESVSFLGINLIHWYQQKPG
QPPKLLIYSASNLQSGVPARFSGSGSGTDFTLTISSVEPEDTANYYCLQSRTLPRTFGQGTKVE
IKGSTSGSGKPGSGEGSTKGQIQLVQSGPELKKPGGSVKISCKASGYTFTSYSINWVRQAPGKG
LEWVGWINTETREPAYAQGFTGRFTFSADTSKSMAYLQINSLRAEDTAVYYCALDYLYSLDFWG
QGTLVTVSSGGGGSQVQLVQSGAEVKKPGSSVKVSCKASGYKFTSYVMHWVKQAPGQGLEWIGY
INPYNDVTKYNEKFKGKATLTADESTNTAYMELSSLRSEDTAVHYCARGSYYDYDGFVYWGQGT
LVTVSSGGGGSGGGGSGGGGSGGGGSDIQMTQSPSTLSASVGDRVTMTCSATSSVSYMHWYQQK
PGKAPKRWIYDTSKLASGVPARFIGSGSGTEFTLTISSLQPDDFATYYCQQWSSNPLTFGGGTK
VEIKHHHHHH
SEQ ID NO: 85 (AA, Diplodocus-11)
MGWSCIILFLVATATGVHSDIVLTQSPASLAVSPGQRATITCRASESVSFLGINLIHWYQQKPG
QPPKLLIYSASNLQSGVPARFSGSGSGTDFTLTISSVEPEDTANYYCLQSRTLPRTFGQGTKVE
IKGGGGSGGGGSGGGGSQIQLVQSGPELKKPGGSVKISCKASGYTFTSYSINWVRQAPGKGLEW
VGWINTETREPAYAQGFTGRFTFSADTSKSMAYLQINSLRAEDTAVYYCALDYLYSLDFWGQGT
LVTVSSGGGGSQVQLVESGGGVVQPGRSLRLSCSSSGYTETRYTMHWVRQAPGKGLEWIGINPS
RGYTNYNQKVKDRFTISTDKSKSTAFLQMDSLRPEDTAVYYCARYYDDHYCLDYWGQGTPVTVS
SGGGGSGGGGSGGGGSGGGGSDIQMTQSPSSLSASVGDRVTITCSASSSVSYMNWYQQTPGKAP
KRWIYDTSKLASGVPSRFSGSGSGTDYTFTISSLQPEDIATYYCQQWSSNPFTFGQGTKLQITR
HHHHHH
SEQ ID NO: 86 (AA, Diplodocus-12)
MGWSCIILFLVATATGVHSDIVLTQSPASLAVSPGQRATITCRASESVSFLGINLIHWYQQKPG
QPPKLLIYSASNLQSGVPARFSGSGSGTDFTLTISSVEPEDTANYYCLQSRTLPRTFGQGTKVE
IKGSTSGSGKPGSGEGSTKGQIQLVQSGPELKKPGGSVKISCKASGYTFTSYSINWVRQAPGKG
LEWVGWINTETREPAYAQGFTGRFTFSADTSKSMAYLQINSLRAEDTAVYYCALDYLYSLDFWG
QGTLVTVSSGGGGSQVQLQQSGAELARPGASVKMSCKTSGYTFTRYTMHWVKQRPGQGLEWIGY
INPSRGYTNYNQKFKDKATLTTDKSSSTAYMQLSSLTSEDSAVYYCARYYDDHYSLDYWGQGTT
LTVSSGGGGSGGGGSGGGGSGGGGSQIVLTQSPAIMSASPGEKVTMTCRASSSVSYMNWYQQKS
GTSPKRWIYDTSKVASGVPYRFSGSGSGTSYSLTISSMEAEDAATYYCQQWSSNPLTFGAGTKL
ELKSSGGSGGSGGSGGSGGSGGSGGSGGSGGSGSKWVTFISLLFLFSSAYSRGVFRRDAHKSEV
AHRFKDLGEENFKALVLIAFAQYLQQCPFEDHVKLVNEVTEFAKTCVADESAENCDKSLHTLFG
DKLCTVATLRETYGEMADCCAKQEPERNECFLQHKDDNPNLPRLVRPEVDVMCTAFHDNEETFL
KKYLYEIARRHPYFYAPELLFFAKRYKAAFTECCQAADKAACLLPKLDELRDEGKASSAKQRLK
CASLQKFGERAFKAWAVARLSQRFPKAEFAEVSKLVTDLTKVHTECCHGDLLECADDRADLAKY
ICENQDSISSKLKECCEKPLLEKSHCIAEVENDEMPADLPSLAADFVESKDVCKNYAEAKDVFL
GMFLYEYARRHPDYSVVLLLRLAKTYKTTLEKCCAAADPHECYAKVFDEFKPLVEEPQNLIKQN
CELFEQLGEYKFQNALLVRYTKKVPQVSTPTLVEVSRNLGKVGSKCCKHPEAKRMPCAEDYLSV
VLNQLCVLHEKTPVSDRVTKCCTESLVNRRPCFSALEVDETYVPKEFNAETFTFHADICTLSEK
ERQIKKQTALVELVKHKPKATKEQLKAVMDDFAAFVEKCCKADDKETCFAEEGKKLVAASRAAL
GLHHHHHH
SEQ ID NO: 87 (AA, Diplodocus-13)
MGWSCIILFLVATATGVHSDIVLTQSPASLAVSPGQRATITCRASESVSFLGINLIHWYQQKPG
QPPKLLIYSASNLQSGVPARFSGSGSGTDFTLTISSVEPEDTANYYCLQSRTLPRTFGQGTKVE
IKGSTSGSGKPGSGEGSTKGQIQLVQSGPELKKPGGSVKISCKASGYTFTSYSINWVRQAPGKG
LEWVGWINTETREPAYAQGFTGRFTFSADTSKSMAYLQINSLRAEDTAVYYCALDYLYSLDFWG
QGTLVTVSSGSGGSGGSGGSGGSGGSGGSGGSGGSGGSGSKWVTFISLLFLFSSAYSRGVFRRD
AHKSEVAHRFKDLGEENFKALVLIAFAQYLQQCPFEDHVKLVNEVTEFAKTCVADESAENCDKS
LHTLFGDKLCTVATLRETYGEMADCCAKQEPERNECFLQHKDDNPNLPRLVRPEVDVMCTAFHD
NEETFLKKYLYEIARRHPYFYAPELLFFAKRYKAAFTECCQAADKAACLLPKLDELRDEGKASS
AKQRLKCASLQKFGERAFKAWAVARLSQRFPKAEFAEVSKLVTDLTKVHTECCHGDLLECADDR
ADLAKYICENQDSISSKLKECCEKPLLEKSHCIAEVENDEMPADLPSLAADFVESKDVCKNYAE
AKDVFLGMFLYEYARRHPDYSVVLLLRLAKTYKTTLEKCCAAADPHECYAKVFDEFKPLVEEPQ
NLIKQNCELFEQLGEYKFQNALLVRYTKKVPQVSTPTLVEVSRNLGKVGSKCCKHPEAKRMPCA
EDYLSVVLNQLCVLHEKTPVSDRVTKCCTESLVNRRPCFSALEVDETYVPKEFNAETFTFHADI
CTLSEKERQIKKQTALVELVKHKPKATKEQLKAVMDDFAAFVEKCCKADDKETCFAEEGKKLVA
ASRAALGLSSGGSGGSGGSGGSGGSGGSGGSGGSGGSGSQVQLQQSGAELARPGASVKMSCKTS
GYTFTRYTMHWVKQRPGQGLEWIGYINPSRGYTNYNQKFKDKATLTTDKSSSTAYMQLSSLTSE
DSAVYYCARYYDDHYSLDYWGQGTTLTVSSGGGGSGGGGSGGGGSGGGGSQIVLTQSPAIMSAS
PGEKVTMTCRASSSVSYMNWYQQKSGTSPKRWIYDTSKVASGVPYRFSGSGSGTSYSLTISSME
AEDAATYYCQQWSSNP LTFGAGTKLELKGGSHHHHHH
SEQ ID NO: 88 (AA, Trilobyte-1)
MGWSCIILFLVATATGVHSDYKDDDDKDIVLTQSPASLAVSPGQRATITCRASESVSFLGINLI
HWYQQKPGQPPKLLIYSASNLQSGVPARFSGSGSGTDFTLTISSVEPEDTANYYCLQSRTLPRT
FGQGTKVEIKGSTSGSGKPGSGEGSTKGQIQLVQSGPELKKPGGSVKISCKASGYTFTSYSINW
VRQAPGKGLEWVGWINTETREPAYAQGFTGRFTFSADTSKSMAYLQINSLRAEDTAVYYCALDY
LYSLDFWGQGTLVTVSSGGGGSQVQLQQSGAELARPGASVKMSCKTSGYTFTRYTMHWVKQRPG
QGLEWIGYINPSRGYTNYNQKFKDKATLITDKSSSTAYMQLSSLTSEDSAVYYCARYYDDHYSL
DYWGQGTTLTVSSGGGGSGGGGSGGGGSGGGGSQIVLTQSPAIMSASPGEKVTMTCRASSSVSY
MNWYQQKSGTSPKRWIYDTSKVASGVPYRFSGSGSGTSYSLTISSMEAEDAATYYCQQWSSNPL
TFGAGTKLELKSSGGSGGSGGSGGSGGGGSGGSGGSGGSGGSGSDIVLTQSPASLAVSPGQRAT
ITCRASESVSFLGINLIHWYQQKPGQPPKLLIYSASNLQSGVPARFSGSGSGTDFTLTISSVEP
EDTANYYCLQSRTLPRTFGQGTKVEIKGSTSGSGKPGSGEGSTKGQIQLVQSGPELKKPGGSVK
ISCKASGYTFTSYSINWVRQAPGKGLEWVGWINTETREPAYAQGFTGRFTFSADTSKSMAYLQI
NSLRAEDTAVYYCALDYLYSLDFWGQGTLVTVSSGGGGSQVQLQQSGAELARPGASVKMSCKTS
GYTFTRYTMHWVKQRPGQGLEWIGYINPSRGYTNYNQKFKDKATLTTDKSSSTAYMQLSSLTSE
DSAVYYCARYYDDHYSLDYWGQGTTLTVSSGGGGSGGGGSGGGGSGGGGSQIVLTQSPAIMSAS
PGEKVTMTCRASSSVSYMNWYQQKSGTSPKRWIYDTSKVASGVPYRFSGSGSGTSYSLTISSME
AEDAATYYCQQWSSNPLTFGAGTKLELKGGSHHHHHH
SEQ ID NO: 89 (AA, Trilobyte-2)
MGWSCIILFLVATATGVHSDYKDDDDKQIQLVQSGPELKKPGGSVKISCKASGYTFTSYSINWV
RQAPGKGLEWVGWINTETREPAYAQGFTGRFTFSADTSKSMAYLQINSLRAEDTAVYYCALDYL
YSLDFWGQGTLVTVSSGSTSGSGKPGSGEGSTKGDIVLTQSPASLAVSPGQRATITCRASESVS
FLGINLIHWYQQKPGQPPKLLIYSASNLQSGVPARFSGSGSGTDFTLTISSVEPEDTANYYCLQ
SRTLPRTFGQGTKVEIKSSGGSGGSGGSGGSGGSGGSGGSGGSGGSGSDIVLTQSPASLAVSPG
QRATITCRASESVSFLGINLIHWYQQKPGQPPKLLIYSASNLQSGVPARFSGSGSGTDFTLTIS
SVEPEDTANYYCLQSRTLPRTFGQGTKVEIKGSTSGSGKPGSGEGSTKGQIQLVQSGPELKKPG
GSVKISCKASGYTFTSYSINWVRQAPGKGLEWVGWINTETREPAYAQGFTGRFTFSADTSKSMA
YLQINSLRAEDTAVYYCALDYLYSLDFWGQGTLVTVSSGGGGSQVQLQQSGAELARPGASVKMS
CKTSGYTFTRYTMHWVKQRPGQGLEWIGYINPSRGYTNYNQKFKDKATLTTDKSSSTAYMQLSS
LTSEDSAVYYCARYYDDHYSLDYWGQGTTLTVSSGGGGSGGGGSGGGGSGGGGSQIVLTQSPAI
MSASPGEKVTMTCRASSSVSYMNWYQQKSGTSPKRWIYDTSKVASGVPYRFSGSGSGTSYSLTI
SSMEAEDAATYYCQQWSSNPLTFGAGTKLELKSSGGSGGSGGSGGSGGGGSGGSGGSGGSGGSG
SQIVLTQSPAIMSASPGEKVTMTCRASSSVSYMNWYQQKSGTSPKRWIYDTSKVASGVPYRFSG
SGSGTSYSLTISSMEAEDAATYYCQQWSSNPLTFGAGTKLELKGGGGSGGGGSGGGGSGGGGSQ
VQLQQSGAELARPGASVKMSCKTSGYTFTRYTMHWVKQRPGQGLEWIGYINPSRGYTNYNQKFK
DKATLTTDKSSSTAYMQLSSLTSEDSAVYYCARYYDDHYSLDYWGQGTTLTVSSGGSHHHHHH
SEQ ID NO: 90 (AA, Trilobyte-3)
MGWSCIILFLVATATGVHSDYKDDDDKQIQLVQSGPELKKPGGSVKISCKASGYTFTSYSINWV
RQAPGKGLEWVGWINTETREPAYAQGFTGRFTFSADTSKSMAYLQINSLRAEDTAVYYCALDYL
YSLDFWGQGTLVTVSSGSTSGSGKPGSGEGSTKGDIVLTQSPASLAVSPGQRATITCRASESVS
FLGINLIHWYQQKPGQPPKLLIYSASNLQSGVPARFSGSGSGTDFTLTISSVEPEDTANYYCLQ
SRTLPRTFGQGTKVEIKSSGGSGGSGGSGGSGGSGGSGGSGGSGGSGSDIVLTQSPASLAVSPG
QRATITCRASESVSFLGINLIHWYQQKPGQPPKLLIYSASNLQSGVPARFSGSGSGTDFTLTIS
SVEPEDTANYYCLQSRTLPRTFGQGTKVEIKGSTSGSGKPGSGEGSTKGQIQLVQSGPELKKPG
GSVKISCKASGYTFTSYSINWVRQAPGKGLEWVGWINTETREPAYAQGFTGRFTFSADTSKSMA
YLQINSLRAEDTAVYYCALDYLYSLDFWGQGTLVTVSSSSGGSGGSGGSGGSGGGGSGGSGGSG
GSGGSGSQVQLQQSGAELARPGASVKMSCKTSGYTFTRYTMHWVKQRPGQGLEWIGYINPSRGY
TNYNQKFKDKATLTTDKSSSTAYMQLSSLTSEDSAVYYCARYYDDHYSLDYWGQGTTLTVSSGG
GGSGGGGSGGGGSGGGGSQIVLTQSPAIMSASPGEKVTMTCRASSSVSYMNWYQQKSGTSPKRW
IYDTSKVASGVPYRFSGSGSGTSYSLTISSMEAEDAATYYCQQWSSNPLTFGAGTKLELKSSGG
SGGSGGSGGSGGGGSGGSGGSGGSGGSGSQIVLTQSPAIMSASPGEKVTMTCRASSSVSYMNWY
QQKSGTSPKRWIYDTSKVASGVPYRFSGSGSGTSYSLTISSMEAEDAATYYCQQWSSNPLTFGA
GTKLELKGGGGSGGGGSGGGGSGGGGSQVQLQQSGAELARPGASVKMSCKTSGYTFTRYTMHWV
KQRPGQGLEWIGYINPSRGYTNYNQKFKDKATLTTDKSSSTAYMQLSSLTSEDSAVYYCARYYD
DHYSLDYWGQGTTLTVSSGGSHHHHHH
SEQ ID NO: 91 (AA, Trilobyte-4)
MGWSCIILFLVATATGVHSDYKDDDDKDIVLTQSPASLAVSPGQRATITCRASESVSFLGINLI
HWYQQKPGQPPKLLIYSASNLQSGVPARFSGSGSGTDFTLTISSVEPEDTANYYCLQSRTLPRT
FGQGTKVEIKGSTSGSGKPGSGEGSTKGQIQLVQSGPELKKPGGSVKISCKASGYTFTSYSINW
VRQAPGKGLEWVGWINTETREPAYAQGFTGRFTFSADTSKSMAYLQINSLRAEDTAVYYCALDY
LYSLDFWGQGTLVTVSSGGGGSQVQLQQSGAELARPGASVKMSCKTSGYTFTRYTMHWVKQRPG
QGLEWIGYINPSRGYTNYNQKFKDKATLTTDKSSSTAYMQLSSLTSEDSAVYYCARYYDDHYSL
DYWGQGTTLTVSSGGGGSGGGGSGGGGSGGGGSQIVLTQSPAIMSASPGEKVTMTCRASSSVSY
MNWYQQKSGTSPKRWIYDTSKVASGVPYRFSGSGSGTSYSLTISSMEAEDAATYYCQQWSSNPL
TFGAGTKLELKSSGGSGGSGGSGGSGGSGGSGGSGGSGGSGSDIVLTQSPASLAVSPGQRATIT
CRASESVSFLGINLIHWYQQKPGQPPKLLIYSASNLQSGVPARFSGSGSGTDFTLTISSVEPED
TANYYCLQSRTLPRTFGQGTKVEIKGSTSGSGKPGSGEGSTKGQIQLVQSGPELKKPGGSVKIS
CKASGYTFTSYSINWVRQAPGKGLEWVGWINTETREPAYAQGFTGRFTFSADTSKSMAYLQINS
LRAEDTAVYYCALDYLYSLDFWGQGTLVTVSSGGSHHHHHH
SEQ ID NO: 92 (AA, Trilobyte-5)
MGWSCIILFLVATATGVHSDYKDDDDKDIVLTQSPASLAVSPGQRATITCRASESVSFLGINLI
HWYQQKPGQPPKLLIYSASNLQSGVPARFSGSGSGTDFTLTISSVEPEDTANYYCLQSRTLPRT
FGQGTKVEIKGSTSGSGKPGSGEGSTKGQIQLVQSGPELKKPGGSVKISCKASGYTFTSYSINW
VRQAPGKGLEWVGWINTETREPAYAQGFTGRFTFSADTSKSMAYLQINSLRAEDTAVYYCALDY
LYSLDFWGQGTLVTVSSGGGGSQVQLQQSGAELARPGASVKMSCKTSGYTFTRYTMHWVKQRPG
QGLEWIGYINPSRGYTNYNQKFKDKATLTTDKSSSTAYMQLSSLTSEDSAVYYCARYYDDHYSL
DYWGQGTTLTVSSGGGGSGGGGSGGGGSGGGGSQIVLTQSPAIMSASPGEKVTMTCRASSSVSY
MNWYQQKSGTSPKRWIYDTSKVASGVPYRFSGSGSGTSYSLTISSMEAEDAATYYCQQWSSNPL
TFGAGTKLELKSSGGSGGSGGSGGSGGSGGSGGSGGSGGSGSDIVLTQSPASLAVSPGQRATIT
CRASESVTILGSHLIHWYQQKPGQPPKLLINLASNVNTGVPARFSGSGSGTDFTLTISSVEPED
TANYYCLQSRTLPRTFGQGTKVEIKGSTSGSGKPGSGEGSTKGQIQLVQSGPELKKPGGSVKIS
CKASGYTFTDYSINWVRQAPGKGLEWVGWINTETREPAYAYDFTGRFTFSADTSKSMAYLQINS
LRAEDTAVYYCALDYTYGMDYWGQGTLVTVSSGGSHHHHHH
SEQ ID NO: 93 (AA, Trilobyte-6)
MGWSCIILFLVATATGVHSDYKDDDDKQIQLVQSGPELKKPGGSVKISCKASGYTFTSYSINWV
RQAPGKGLEWVGWINTETREPAYAQGFTGRFTFSADTSKSMAYLQINSLRAEDTAVYYCALDYL
YSLDFWGQGTLVTVSSGSTSGSGKPGSGEGSTKGDIVLTQSPASLAVSPGQRATITCRASESVS
FLGINLIHWYQQKPGQPPKLLIYSASNLQSGVPARFSGSGSGTDFTLTISSVEPEDTANYYCLQ
SRTLPRTFGQGTKVEIKSSGGSGGSGGSGGSGGGGSGGSGGSGGSGGSGSDIVLTQSPASLAVS
PGQRATITCRASESVTILGSHLIHWYQQKPGQPPKLLINLASNVNTGVPARFSGSGSGTDFTLT
ISSVEPEDTANYYCLQSRTLPRTFGQGTKVEIKGSTSGSGKPGSGEGSTKGQIQLVQSGPELKK
PGGSVKISCKASGYTFTDYSINWVRQAPGKGLEWVGWINTETREPAYAYDFTGRFTFSADTSKS
MAYLQINSLRAEDTAVYYCALDYTYGMDYWGQGTLVTVSSSGGGGSQVQLQQSGAELARPGASV
KMSCKTSGYTFTRYTMHWVKQRPGQGLEWIGYINPSRGYTNYNQKFKDKATLTTDKSSSTAYMQ
LSSLTSEDSAVYYCARYYDDHYSLDYWGQGTTLTVSSGGGGSGGGGSGGGGSGGGGSQIVLTQS
PAIMSASPGEKVTMTCRASSSVSYMNWYQQKSGTSPKRWIYDTSKVASGVPYRFSGSGSGTSYS
LTISSMEAEDAATYYCQQWSSNPLTFGAGTKLELKSSGGSGGSGGSGGSGGGGSGGSGGSGGSG
GSGSQIVLTQSPAIMSASPGEKVTMTCRASSSVSYMNWYQQKSGTSPKRWIYDTSKVASGVPYR
FSGSGSGTSYSLTISSMEAEDAATYYCQQWSSNPLTFGAGTKLELKGGGGSGGGGSGGGGSGGG
GSQVQLQQSGAELARPGASVKMSCKTSGYTFTRYTMHWVKQRPGQGLEWIGYINPSRGYTNYNQ
KFKDKATLTTDKSSSTAYMQLSSLTSEDSAVYYCARYYDDHYSLDYWGQGTTLTVSSGGSHHHH
HH
SEQ ID NO: 94 (AA, Trilobyte-7)
MGWSCIILFLVATATGVHSDYKDDDDKQIQLVQSGPELKKPGGSVKISCKASGYTFTSYSINWV
RQAPGKGLEWVGWINTETREPAYAQGFTGRFTFSADTSKSMAYLQINSLRAEDTAVYYCALDYL
YSLDFWGQGTLVTVSSGGGSGGGGSGGGGSGGGGSDIVLTQSPASLAVSPGQRATITCRASESV
SFLGINLIHWYQQKPGQPPKLLIYSASNLQSGVPARFSGSGSGTDFTLTISSVEPEDTANYYCL
QSRTLPRTFGQGTKVEIKSSGGSGGSGGSGGSGGGGSGGSGGSGGSGGSGSDIVLTQSPASLAV
SPGQRATITCRASESVTILGSHLIHWYQQKPGQPPKLLINLASNVNTGVPARFSGSGSGTDFTL
TISSVEPEDTANYYCLQSRTLPRTFGQGTKVEIKGSTSGSGKPGSGEGSTKGQIQLVQSGPELK
KPGGSVKISCKASGYTFTDYSINWVRQAPGKGLEWVGWINTETREPAYAYDFTGRFTFSADTSK
SMAYLQINSLRAEDTAVYYCALDYTYGMDYWGQGTLVTVSSSGGGGSQVQLQQSGAELARPGAS
VKMSCKTSGYTFTRYTMHWVKQRPGQGLEWIGYINPSRGYTNYNQKFKDKATLTTDKSSSTAYM
QLSSLTSEDSAVYYCARYYDDHYSLDYWGQGTTLTVSSGGGGSGGGGSGGGGSGGGGSQIVLTQ
SPAIMSASPGEKVTMTCRASSSVSYMNWYQQKSGTSPKRWIYDTSKVASGVPYRFSGSGSGTSY
SLTISSMEAEDAATYYCQQWSSNPLTFGAGTKLELKSSGGSGGSGGSGGSGGGGSGGSGGSGGS
GGSGSDIQLTQSPAIMSASPGEKVTMTCRASSSVSYMNWYQQKSGTSPKRWIYDTSKVASGVPY
RFSGSGSGTSYSLTISSMEAEDAATYYCQQWSSNPLTFGAGTKLELKVEGGSGGSGGSGGSGGV
DDIKLQQSGAELARPGASVKMSCKTSGYTFTRYTMHWVKQRPGQGLEWIGYINPSRGYTNYNQK
FKDKATLTTDKSSSTAYMQLSSLTSEDSAVYYCARYYDDHYCLDYWGQGTTLTVSSGGSHHHHH
H
SEQ ID NO: 95 (AA, Trilobyte-14)
MGWSCIILFLVATATGVHSDYKDDDDKTRVDQTPRTATRETGESLTINCVLTDTSYPLYSTYWY
RKNPGSSNKEQISISGRYVESVNKGTKSFSLRIKDLTVADSATYICRAMGTNIWTGDGAGTVLT
VNSSGGSGGSGGSGGSGGSGGSGGSGGSGGSGSDIVLTQSPASLAVSPGQRATITCRASESVSF
LGINLIHWYQQKPGQPPKLLIYSASNLQSGVPARFSGSGSGTDFTLTISSVEPEDTANYYCLQS
RTLPRTFGQGTKVEIKGSTSGSGKPGSGEGSTKGQIQLVQSGPELKKPGGSVKISCKASGYTFT
SYSINWVRQAPGKGLEWVGWINTETREPAYAQGFTGRFTFSADTSKSMAYLQINSLRAEDTAVY
YCALDYLYSLDFWGQGTLVTVSSGGGGSQVQLQQSGAELARPGASVKMSCKTSGYTFTRYTMHW
VKQRPGQGLEWIGYINPSRGYTNYNQKFKDKATLTTDKSSSTAYMQLSSLTSEDSAVYYCARYY
DDHYSLDYWGQGTTLTVSSGGGGSGGGGSGGGGSGGGGSQIVLTQSPAIMSASPGEKVTMTCRA
SSSVSYMNWYQQKSGTSPKRWIYDTSKVASGVPYRFSGSGSGTSYSLTISSMEAEDAATYYCQQ
WSSNPLTFGAGTKLELKHHHHHH
SEQ ID NO: 96 (AA, Trilobyte-15)
MGWSCIILFLVATATGVHSDYKDDDDKDIVLTQSPASLAVSPGQRATITCRASESVSFLGINLI
HWYQQKPGQPPKLLIYSASNLQSGVPARFSGSGSGTDFTLTISSVEPEDTANYYCLQSRTLPRT
FGQGTKVEIKGSTSGSGKPGSGEGSTKGQIQLVQSGPELKKPGGSVKISCKASGYTFTSYSINW
VRQAPGKGLEWVGWINTETREPAYAQGFTGRFTFSADTSKSMAYLQINSLRAEDTAVYYCALDY
LYSLDFWGQGTLVTVSSGSGGSGGSGGSGGSGGSGGSGGSGGSGGSGSTRVDQTPRTATRETGE
SLTINCVLIDTSYPLYSTYWYRKNPGSSNKEQISISGRYVESVNKGTKSFSLRIKDLTVADSAT
YICRAMGTNIWTGDGAGTVLTVNSSGGSGGSGGSGGSGGGGSGGSGGSGGSGGSGSQVQLQQSG
AELARPGASVKMSCKTSGYTFTRYTMHWVKQRPGQGLEWIGYINPSRGYTNYNQKFKDKATLTT
DKSSSTAYMQLSSLTSEDSAVYYCARYYDDHYSLDYWGQGTTLTVSSGGGGSGGGGSGGGGSGG
GGSQIVLTQSPAIMSASPGEKVTMTCRASSSVSYMNWYQQKSGTSPKRWIYDTSKVASGVPYRF
SGSGSGTSYSLTISSMEAEDAATYYCQQWSSNPLTFGAGTKLELKGGSHHHHHH
SEQ ID NO: 97 (AA, Trilobyte-16)
MGWSCIILFLVATATGVHSDYKDDDDKDIVLTQSPASLAVSPGQRATITCRASESVSFLGINLI
HWYQQKPGQPPKLLIYSASNLQSGVPARFSGSGSGTDFTLTISSVEPEDTANYYCLQSRTLPRT
FGQGTKVEIKGSTSGSGKPGSGEGSTKGQIQLVQSGPELKKPGGSVKISCKASGYTFTSYSINW
VRQAPGKGLEWVGWINTETREPAYAQGFTGRFTFSADTSKSMAYLQINSLRAEDTAVYYCALDY
LYSLDFWGQGTLVTVSSGGGGSQVQLQQSGAELARPGASVKMSCKTSGYTFTRYTMHWVKQRPG
QGLEWIGYINPSRGYTNYNQKFKDKATLTTDKSSSTAYMQLSSLTSEDSAVYYCARYYDDHYSL
DYWGQGTTLTVSSGGGGSGGGGSGGGGSGGGGSQIVLTQSPAIMSASPGEKVTMTCRASSSVSY
MNWYQQKSGTSPKRWIYDTSKVASGVPYRFSGSGSGTSYSLTISSMEAEDAATYYCQQWSSNPL
TFGAGTKLELKSSGGSGGSGGSGGSGGSGGSGGSGGSGGSGSTRVDQTPRTATRETGESLTINC
VLTDTSYPLYSTYWYRKNPGSSNKEQISISGRYVESVNKGTKSFSLRIKDLTVADSATYICRAM
GTNIWTGDGAGTVLTVNHHHHHH
SEQ ID NO: 98 (AA, Trilobyte-17)
MGWSCIILFLVATATGVHSDYKDDDDKDIVLTQSPASLAVSPGQRATITCRASESVSFLGINLI
HWYQQKPGQPPKLLIYSASNLQSGVPARFSGSGSGTDFTLTISSVEPEDTANYYCLQSRTLPRI
FGQGTKVEIKGSTSGSGKPGSGEGSTKGQIQLVQSGPELKKPGGSVKISCKASGYTFTSYSINW
VRQAPGKGLEWVGWINTETREPAYAQGFTGRFTFSADTSKSMAYLQINSLRAEDTAVYYCALDY
LYSLDFWGQGTLVTVSSGGGGSQVQLQQSGAELARPGASVKMSCKTSGYTFTRYTMHWVKQRPG
QGLEWIGYINPSRGYTNYNQKFKDKATLTTDKSSSTAYMQLSSLTSEDSAVYYCARYYDDHYSL
DYWGQGTTLTVSSGGGGSGGGGSGGGGSGGGGSQIVLTQSPAIMSASPGEKVTMTCRASSSVSY
MNWYQQKSGTSPKRWIYDTSKVASGVPYRFSGSGSGTSYSLTISSMEAEDAATYYCQQWSSNPL
TFGAGTKLELKSSGGSGGSGGSGGSGGSGGSGGSGGSGGSGSEPKSCDKTHTCPPCPAPELLGG
PSVFLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMH
EALHNHYTQKSLSLSPGKHHHHHH
SEQ ID NO: 99 (AA, Trilobyte-18)
MGWSCIILFLVATATGVHSDYKDDDDKDIVLTQSPASLAVSPGQRATITCRASESVSFLGINLI
HWYQQKPGQPPKLLIYSASNLQSGVPARFSGSGSGTDFTLTISSVEPEDTANYYCLQSRTLPRT
FGQGTKVEIKGSTSGSGKPGSGEGSTKGQIQLVQSGPELKKPGGSVKISCKASGYTFTSYSINW
VRQAPGKGLEWVGWINTETREPAYAQGFTGRFTFSADTSKSMAYLQINSLRAEDTAVYYCALDY
LYSLDFWGQGTLVTVSSGGGGSQVQLQQSGAELARPGASVKMSCKTSGYTFTRYTMHWVKQRPG
QGLEWIGYINPSRGYTNYNQKFKDKATLTTDKSSSTAYMQLSSLTSEDSAVYYCARYYDDHYSL
DYWGQGTTLTVSSGGGGSGGGGSGGGGSGGGGSQIVLTQSPAIMSASPGEKVTMTCRASSSVSY
MNWYQQKSGTSPKRWIYDTSKVASGVPYRFSGSGSGTSYSLTISSMEAEDAATYYCQQWSSNPL
TFGAGTKLELKSSGGSGGSGGSGGSGGSGGSGGSGGSGGSGSDIQMTQSPSSVSASVGDRVTIT
CQSSPSVWSNFLSWYQQKPGKAPKLLIYEASKLTSGVPSRFSGSGSGTDFTLTISSLQPEDFAT
YYCGGGYSSISDTTFGGGTKVEIKGGGGSGGGGSGGGGSGGGGSEVQLLESGGGLVQPGGSLRL
SCAVSGIDLSNYAINWVRQAPGKGLEWIGIIWASGTTFYATWAKGRFTISRDNSKNTVYLQMNS
LRAEDTAVYYCARTVPGYSTAPYFDLWGQGTLVTVSSHHHHHH
SEQ ID NO: 100 (AA, Trilobyte-19)
MGWSCIILFLVATATGVHSDYKDDDDKDIVLTQSPASLAVSPGQRATITCRASESVSFLGINLI
HWYQQKPGQPPKLLIYSASNLQSGVPARFSGSGSGTDFTLTISSVEPEDTANYYCLQSRTLPRT
FGQGTKVEIKGSTSGSGKPGSGEGSTKGQIQLVQSGPELKKPGGSVKISCKASGYTFTSYSINW
VRQAPGKGLEWVGWINTETREPAYAQGFTGRFTFSADTSKSMAYLQINSLRAEDTAVYYCALDY
LYSLDFWGQGTLVTVSSGSGGSGGSGGSGGSGGSGGSGGSGGSGGSGSEVQLLESGGGLVQPGG
SLRLSCAVSGIDLSNYAINWVRQAPGKGLEWIGIIWASGTTFYATWAKGRFTISRDNSKNTVYL
QMNSLRAEDTAVYYCARTVPGYSTAPYFDLWGQGTLVTVSSGGGGSGGGGSGGGGSGGGGSDIQ
MTQSPSSVSASVGDRVTITCQSSPSVWSNFLSWYQQKPGKAPKLLIYEASKLTSGVPSRFSGSG
SGTDFTLTISSLQPEDFATYYCGGGYSSISDTTFGGGTKVEIKSSGGSGGSGGSGGSGGGGSGG
SGGSGGSGGSGSQIVLTQSPAIMSASPGEKVTMTCRASSSVSYMNWYQQKSGTSPKRWIYDTSK
VASGVPYRFSGSGSGTSYSLTISSMEAEDAATYYCQQWSSNPLTFGAGTKLELKGGGGSGGGGS
GGGGSGGGGSQVQLQQSGAELARPGASVKMSCKTSGYTFTRYTMHWVKQRPGQGLEWIGYINPS
RGYTNYNQKFKDKATLTTDKSSSTAYMQLSSLTSEDSAVYYCARYYDDHYSLDYWGQGTTLTVS
SGGSHHHHHH
SEQ ID NO: 101 (AA, Trilobyte-299 bispecific antibody)
MYRMQLLSCIALSLALVINSDIVLTQSPASLAVSPGQRATISCRASESVSFLGINLIHWYQQKP
GQPPKLLIYLASNLETGVPARFSGSGSGTDFTLTISSVEPEDTANYYCLQSRTLPRTFGQGTKV
EIKGSTSGSGKPGSGEGSTKGQIQLVQSGPELKKPGGSVKISCKASGYTFTSYSINWVRQAPGK
GLEWVGWINTETREPAYAQGFTGRFTFSADTSKSMAYLQINSLRAEDTAVYYCALDYLYSLDFW
GQGTLVTVSSGGGGSQVQLVQSGAEVKKPGASVKVSCKASGYTFTRYTMHWVRQAPGQGLEWMG
YINPSKGYTNYNQKFKGRVTITADKSTSTAYMELSSLRSEDTAVYYCARYYDDHYSLDYWGQGT
LVTVSSGGGGSGGGGSGGGGSGGGGSEIVMTQSPAILSVSPGERATLSCRASSSVSYLNWYQQK
PGTAPKRWIYDTSKVASGVPYRFSGSGSGTDFILTISSLQAEDEATYYCQQWSSNPLTFGQGTK
LEIKSSGGSGGSGGSGGSGGSGGSGGSGGSGGSGSDIVLTQSPASLAVSPGQRATISCRASESV
SFLGINLIHWYQQKPGQPPKLLIYLASNLETGVPARFSGSGSGTDFTLTISSVEPEDTANYYCL
QSRTLPRTFGQGTKVEIKGSTSGSGKPGSGEGSTKGQIQLVQSGPELKKPGGSVKISCKASGYT
FTSYSINWVRQAPGKGLEWVGWINTETREPAYAQGFTGRFTFSADISKSMAYLQINSLRAEDTA
VYYCALDYLYSLDFWGQGTLVTVSS
SEQ ID NO: 102 (AA, CXCR4)
MSIPLPLLQIYTSDNYTEEMGSGDYDSMKEPCFREENANFNKIFLPTIYSIIFLTGIVGNGLVI
LVMGYQKKLRSMTDKYRLHLSVADLLFVITLPFWAVDAVANWYFGNFLCKAVHVIYTVNLYSSV
LILAFISLDRYLAIVHATNSQRPRKLLAEKVVYVGVWIPALLLTIPDFIFANVSEADDRYICDR
FYPNDLWVVVFQFQHIMVGLILPGIVILSCYCIIISKLSHSKGHQKRKALKTIVILILAFFACW
LPYYIGISIDSFILLEIIKQGCEFENTVHKWISITEALAFFHCCLNPILYAFLGAKFKTSAQHA
LTSVSRGSSLKILSKGKRGGHSSVSTESESSSFHSS
SEQ ID NO: 103 (AA, IL-12, partial)
NLPVATPDPGMFPCLHHSQNLLRAVSNMLQKARQTLEFYPCTSEEIDHEDITKDKTSTVEACLP
LELTKNESCLNSRETSFITNGSCLASRKTSFMMALCLSSIYEDLKMYQVEFKTMNAKLIMDPKR
QIFLDQNMLAVIDELMQALNFNSETVPQKSSLEEPDFYKTKIKLCILLHAFRIRAVTIDRVMSY
LNAS
SEQ ID NO: 104 (AA, IL-12B, partial)
IWELKKDVYVVELDWYPDAPGEMVVLTCDTPEEDGITWTLDQSSEVLGSGKILTIQVKEFGDAG
QYTCHKGGEVLSHSLLLLHKKEDGIWSTDILKDQKEPKNKTFLRCEAKNYSGRFTCWWLTTIST
DLTFSVKSSRGSSDPQGVTCGAATLSAERVRGDNKEYEYSVECQEDSACPAAEESLPIEVMVDA
VHKLKYENYTSSFFIRDIIKPDPPKNLQLKPLKNSRQVEVSWEYPDTWSTPHSYFSLTFCVQVQ
GKSKREKKDRVFTDKTSATVICRKNASISVRAQDRYYSSSWSEWASVPCS
SEQ ID NO: 105 (AA, IL-12 dual-subunit fusion protein)
MCHQQLVISWFSLVFLASPLVAIWELKKDVYVVELDWYPDAPGEMVVLTCDTPEEDGITWILDQ
SSEVLGSGKILTIQVKEFGDAGQYTCHKGGEVLSHSLLLLHKKEDGIWSTDILKDQKEPKNKTF
LRCEAKNYSGRFTCWWLTTISTDLTFSVKSSRGSSDPQGVTCGAATLSAERVRGDNKEYEYSVE
CQEDSACPAAEESLPIEVMVDAVHKLKYENYTSSFFIRDIIKPDPPKNLQLKPLKNSRQVEVSW
EYPDTWSTPHSYFSLTFCVQVQGKSKREKKDRVFTDKTSATVICRKNASISVRAQDRYYSSSWS
EWASVPCSGGGGSGGGGSGGGGSGGGGSRNLPVATPDPGMFPCLHHSQNLLRAVSNMLQKARQT
LEFYPCTSEEIDHEDITKDKTSTVEACLPLELTKNESCLNSRETSFIINGSCLASRKTSFMMAL
CLSSIYEDIKMYQVEFKTMNAKLLMDPKRQIFLDQNMLAVIDELMQALNENSETVPQKSSLEEP
DFYKTKIKLCILLHAFRIRAVTIDRVMSYLNAS
SEQ ID NO: 106 (nt, Zeyphyrosaurus bispecific antibody)
AGACCCAAGCUGGCUAGCUCUAAAGAAGCCCCUGGGAGCACAGCUCAUCACCGCCACCAUGUAC
AGGAUGCAACUCCUGUCUUGCAUUGCACUAAGUCUUGCACUUGUCACGAAUUCGGACAUUGUUU
UGACUCAAAGCCCAGCGAGCUUGGCCGUAAGCCCAGGUCAGCGAGCAACAAUAUCUUGCCGAGC
UUCUGAGUCAGUAUCCUUCCUGGGAAUCAAUUUGAUCCAUUGGUAUCAACAAAAGCCAGGGCAA
CCCCCGAAACUGCUUAUAUACCUGGCCUCAAAUUUGGAGACCGGGGUCCCGGCACGAUUUUCAG
GGUCAGGUUCUGGGACCGACUUUACGCUUACGAUUUCUUCCGUCGAACCGGAGGACACCGCUAA
CUAUUAUUGUUUGCAAUCCCGGACCCUUCCACGAACAUUUGGCCAAGGAACAAAAGUUGAAAUC
AAAGGUUCCACUUCAGGAUCUGGCAAGCCUGGCAGCGGAGAAGGCUCAACGAAGGGCCAGAUAC
AGCUCGUCCAGAGUGGCCCCGAGCUGAAAAAACCGGGUGGCAGUGUUAAAAUCAGUUGUAAAGC
GAGCGGAUACACGUUCACGUCAUACUCCAUUAACUGGGUUCGGCAAGCACCAGGAAAGGGUCUU
GAAUGGGUCGGGUGGAUCAACACCGAGACAAGAGAACCUGCCUAUGCCCAAGGGUUCACUGGGA
GGUUCACAUUCUCUGCUGAUACCUCCAAGUCUAUGGCGUACUUGCAAAUUAAUAGUCUCAGAGC
CGAGGACACUGCUGUCUAUUACUGUGCACUCGACUACCUCUAUAGUCUGGACUUUUGGGGUCAG
GGUACUUUGGUCACGGUCUCUUCUGGAGGUGGUGGAUCCCAAGUUCAGUUGGUCCAGUCUGGUG
CCGAGGUUAAAAAACCAGGUGCAUCUGUCAAAGUGAGUUGCAAAGCAUCAGGCUAUACCUUCAC
AAGGUACACAAUGCAUUGGGUGAGGCAGGCCCCUGGGCAGGGCCUCGAAUGGAUGGGAUACAUC
AACCCUUCCAAAGGUUACACCAACUACAAUCAGAAAUUUAAGGGUCGCGUCACCAUUACUGCAG
AUAAAUCUACAAGCACAGCAUAUAUGGAAUUGUCAUCUCUCCGCUCCGAGGAUACCGCCGUCUA
UUACUGUGCAAGGUACUACGAUGACCACUAUAGUCUUGAUUACUGGGGGCAAGGGACAUUGGUU
ACAGUGAGCUCCGGAGGCGGAGGAUCUGGUGGUGGCGGAUCUGGCGGCGGUGGAAGUGGCGGAG
GUGGUAGCGAGAUCGUGAUGACCCAGUCACCCGCUACCUUGUCUGUCUCACCAGGCGAGAGAGC
CACUCUUAGUUGUAGGGCCUCCAGUUCAGUCAGCUAUCUCAACUGGUAUCAGCAGAAGCCCGGC
ACAGCCCCCAAGCGGUGGAUUUAUGACACAAGUAAGGUGGCCAGUGGGGUCCCUUACCGGUUCA
GCGGUUCCGGUAGUGGCACUGAUUUCACACUCACCAUUUCUUCACUUCAAGCAGAAGAUGAGGC
AACUUACUACUGCCAGCAAUGGAGUUCCAAUCCCCUUACAUUCGGUCAGGGGACAAAACUGGAG
AUUAAGUCUAGCGGAGGCUCUGGUGGAUCUGGCGGAAGCGGAGGCAGUGGUGGAUCCGGAGGUA
GCGGCGGAUCUGGCGGUAGUGGAGGUAGUGGAUCUGAUAUCGUACUCACACAGAGUCCUGCUUC
CCUCGCGGUAUCACCUGGACAAAGGGCCACCAUCAGCUGUAGGGCUAGUGAAAGUGUUUCAUUU
CUCGGGAUAAACCUGAUUCACUGGUACCAGCAGAAACCUGGACAGCCACCUAAACUGCUGAUCU
AUCUUGCAAGCAACCUCGAAACAGGAGUACCAGCUAGGUUCAGCGGCAGCGGAUCCGGCACUGA
UUUCACUCUCACCAUAUCCUCUGUUGAGCCAGAAGAUACAGCAAAUUACUACUGCCUCCAGAGU
CGCACUCUGCCUAGGACUUUCGGGCAGGGGACCAAGGUCGAGAUUAAGGGGAGUACGAGUGGUA
GCGGUAAACCAGGAUCUGGCGAGGGAUCCACCAAAGGACAAAUCCAAUUGGUUCAAAGCGGACC
UGAACUCAAGAAGCCAGGAGGGUCAGUGAAGAUUUCCUGCAAGGCUAGUGGGUAUACAUUUACC
UCCUAUAGCAUCAAUUGGGUACGACAGGCUCCGGGGAAAGGUUUGGAGUGGGUGGGCUGGAUUA
AUACUGAAACUCGCGAGCCCGCAUACGCUCAGGGAUUUACGGGUCGCUUUACCUUUUCAGCGGA
CACAAGUAAAAGCAUGGCAUACCUCCAGAUCAACUCACUGCGCGCGGAAGAUACCGCGGUGUAC
UAUUGCGCCCUGGAUUAUCUUUACUCUCUCGAUUUCUGGGGCCAAGGCACGCUCGUGACAGUAU
CCUCCUAGCUAUGAAGAAGGAAGGCAUCCAGACCAGAAACCGAAAAAUGUCUAGCAAAUCCAAA
AAGUGCAAAAAAGUGCAUGACUCACUGGAGGACUUCCCCAAGAACAGCUCGUUUAACCCGGCCG
CCCUCUCCAGACACAUGUCCUCCCUGAGCCACAUCUCGCCCUUCAGCCACUCCAGCCACAUGCU
GACCACGCCCACGCCGAUGCACCCGCCAUCCAGC
SEQ ID NO: 107 (nt, IL12)
AGACCCAAGCUGGCUAGCUCUAAAGAAGCCCCUGGGAGCACAGCUCAUCACCGCCGCCACCAUG
UGUCAUCAACAACUGGUAAUUUCAUGGUUUUCACUCGUAUUCCUGGCAUCACCUCUUGUUGCGA
UCUGGGAACUGAAGAAGGAUGUCUACGUUGUCGAGUUGGAUUGGUAUCCUGAUGCCCCAGGUGA
GAUGGUUGUGUUGACUUGUGAUACACCUGAAGAGGACGGUAUUACUUGGACACUCGACCAGUCC
UCCGAGGUCUUGGGCAGCGGUAAAACCCUUACCAUCCAGGUGAAGGAGUUCGGAGAUGCUGGAC
AGUACACAUGUCAUAAGGGGGGCGAAGUCCUGUCUCACUCCCUCUUGCUGUUGCAUAAAAAGGA
GGACGGGAUCUGGUCUACAGAUAUUCUUAAAGACCAAAAGGAACCCAAAAACAAAACAUUCCUC
AGAUGCGAAGCCAAAAAUUACUCCGGUAGAUUUACAUGUUGGUGGCUUACAACAAUCUCCACAG
ACCUCACAUUCUCCGUCAAAAGCUCCAGAGGAUCAAGCGAUCCACAGGGAGUCACUUGCGGCGC
UGCAACACUCUCCGCAGAGAGAGUGCGCGGCGACAAUAAGGAAUACGAAUACUCUGUUGAGUGU
CAAGAGGAUAGUGCUUGUCCAGCAGCCGAAGAAUCCCUGCCAAUCGAAGUCAUGGUGGACGCUG
UCCACAAGCUGAAAUACGAAAAUUACACCAGUAGUUUCUUUAUCCGCGACAUCAUUAAGCCCGA
CCCUCCAAAGAAUCUUCAGCUUAAACCACUCAAGAACUCAAGACAGGUUGAAGUCUCAUGGGAA
UAUCCAGAUACAUGGUCAACCCCUCAUUCUUAUUUCAGUCUUACUUUUUGUGUGCAGGUUCAAG
GAAAGUCAAAGAGAGAGAAGAAAGACAGAGUUUUUACUGAUAAAACAAGUGCAACAGUGAUCUG
UAGGAAGAAUGCAAGCAUUAGUGUCCGGGCUCAAGACAGGUACUAUAGUAGCUCUUGGUCAGAG
UGGGCAUCAGUGCCAUGUUCAGGCGGAGGCGGAAGCGGCGGAGGCGGAUCCGGAGGCGGAGGCA
GCGGAGGCGGAGGCUCCAGGAACUUGCCCGUUGCAACUCCCGAUCCCGGUAUGUUCCCUUGCCU
GCAUCACUCUCAAAACUUGUUGCGGGCUGUUUCUAACAUGCUCCAGAAGGCAAGGCAGACACUG
GAAUUUUACCCUUGCACAUCCGAAGAGAUCGACCACGAGGACAUUACAAAAGAUAAAACUAGCA
CUGUUGAGGCCUGCUUGCCCCUUGAGUUGACCAAAAACGAGUCCUGUCUGAACUCACGGGAAAC
AUCCUUCAUCACUAAUGGGAGCUGUUUGGCUUCAAGAAAAACAUCCUUCAUGAUGGCCCUUUGC
UUGAGCUCUAUCUACGAGGACUUGAAAAUGUACCAGGUCGAGUUCAAGACAAUGAACGCAAAAC
UGUUGAUGGACCCCAAGAGACAAAUUUUCCUGGAUCAAAAUAUGUUGGCCGUUAUUGACGAGCU
GAUGCAAGCACUGAACUUUAACAGUGAAACAGUGCCUCAGAAAUCUUCCCUGGAGGAACCAGAC
UUUUACAAGACCAAAAUCAAAUUGUGCAUUCUCCUGCACGCCUUCCGGAUCAGAGCAGUGACUA
UUGACCGCGUCAUGUCUUAUCUGAACGCAAGUUGACUAUGAAGAAGGAAGGCAUCCAGACCAGA
AACCGAAAAAUGUCUAGCAAAUCCAAAAAGUGCAAAAAAGUGCAUGACUCACUGGAGGACUUCC
CCAAGAACAGCUCGUUUAACCCGGCCGCCCUCUCCAGACACAUGUCCUCCCUGAGCCACAUCUC
GCCCUUCAGCCACUCCAGCCACAUGCUGACCACGCCCACGCCGAUGCACCCGCCAUCCAGC
SEQ ID NO: 108 (nt, CXCR4)
AGACCCAAGCUGGCUAGCUCUAAAGAAGCCCCUGGGAGCACAGCUCAUCACCGCCGCCACCAUG
UCCAUUCCUUUGCCUCUUUUGCAGAUAUACACUUCAGAUAACUACACCGAGGAAAUGGGCUCAG
GGGACUAUGACUCCAUGAAGGAACCCUGUUUCCGUGAAGAAAAUGCUAAUUUCAAUAAAAUCUU
CCUGCCCACCAUCUACUCCAUCAUCUUCUUAACUGGCAUUGUGGGCAAUGGAUUGGUCAUCCUG
GUCAUGGGUUACCAGAAGAAACUGAGAAGCAUGACGGACAAGUACAGGCUGCACCUGUCAGUGG
CCGACCUCCUCUUUGUCAUCACGCUUCCCUUCUGGGCAGUUGAUGCCGUGGCAAACUGGUACUU
UGGGAACUUCCUAUGCAAGGCAGUCCAUGUCAUCUACACAGUCAACCUCUACAGCAGUGUCCUC
AUCCUGGCCUUCAUCAGUCUGGACCGCUACCUGGCCAUCGUCCACGCCACCAACAGUCAGAGGC
CAAGGAAGCUGUUGGCUGAAAAGGUGGUCUAUGUUGGCGUCUGGAUCCCUGCCCUCCUGCUGAC
UAUUCCCGACUUCAUCUUUGCCAACGUCAGUGAGGCAGAUGACAGAUAUAUCUGUGACCGCUUC
UACCCCAAUGACUUGUGGGUGGUUGUGUUCCAGUUUCAGCACAUCAUGGUUGGCCUUAUCCUGC
CUGGUAUUGUCAUCCUGUCCUGCUAUUGCAUUAUCAUCUCCAAGCUGUCACACUCCAAGGGCCA
CCAGAAGCGCAAGGCCCUCAAGACCACAGUCAUCCUCAUCCUGGCUUUCUUCGCCUGUUGGCUG
CCUUACUACAUUGGGAUCAGCAUCGACUCCUUCAUCCUCCUGGAAAUCAUCAAGCAAGGGUGUG
AGUUUGAGAACACUGUGCACAAGUGGAUUUCCAUCACCGAGGCCCUAGCUUUCUUCCACUGUUG
UCUGAACCCCAUCCUCUAUGCUUUCCUUGGAGCCAAAUUUAAAACCUCUGCCCAGCACGCACUC
ACCUCUGUGAGCAGAGGGUCCAGCCUCAAGAUCCUCUCCAAAGGAAAGCGAGGUGGACAUUCAU
CUGUUUCCACUGAGUCUGAGUCUUCAAGUUUUCACUCCAGCUAACUAUGAAGAAGGAAGGCAUC
CAGACCAGAAACCGAAAAAUGUCUAGCAAAUCCAAAAAGUGCAAAAAAGUGCAUGACUCACUGG
AGGACUUCCCCAAGAACAGCUCGUUUAACCCGGCCGCCCUCUCCAGACACAUGUCCUCCCUGAG
CCACAUCUCGCCCUUCAGCCACUCCAGCCACAUGCUGACCACGCCCACGCCGAUGCACCCGCCA
UCCAGC
SEQ ID NO: 109 (AA, Signal sequence)
MGWSCIILFLVATATGVHS
SEQ ID NO: 110 (AA, VHCD3)
DIKLQQSGAELARPGASVKMSCKTSGYTFTRYTMHWVKQRPGQGLEWIGYINPSRGYTNYNQKF
KDKATLTTDKSSSTAYMQLSSLTSEDSAVYYCARYYDDHYCLDYWGQGTTLTVSS
SEQ ID NO: 111 (AA, VLCD3)
DIQLTQSPAIMSASPGEKVTMTCRASSSVSYMNWYQQKSGTSPKRWIYDTSKVASGVPYRFSGS
GSGTSYSLTISSMEAEDAATYYCQQWSSNPLTFGAGTKLELK
SEQ ID NO: 112 (AA, QKT3-6 VHCD3)
QVQLVESGGGVVQPGRSLRLSCSSSGYTETRYTMHWVRQAPGKGLEWIGINPSRGYTNYNQKVK
DRFTISTDKSKSTAFLQMDSLRPEDTAVYYCARYYDDHYCLDYWGQGTPVTVSS
SEQ ID NO: 113 (AA, QKT3-6 VLCD3)
DIQMTQSPSSLSASVGDRVTITCSASSSVSYMNWYQQTPGKAPKRWIYDTSKLASGVPSRFSGS
GSGTDYTFTISSLQPEDIATYYCQQWSSNPFTFGQGTKLQITR
SEQ ID NO: 114 (AA, LA CD3-VH)
QVQLVQSGAEVKKPGSSVKVSCKASGYKFTSYVMHWVRQAPGQGLEWMGYINPYNDVTKYNEKF
KGRATITADESTNTAYMELSSLRSEDTAFYFCARGSYYDYDGFVYWGQGTLVTVSS
SEQ ID NO: 115 (AA, LA CD3-VL)
DIQMTQSPSTLSASVGDRVTITCSATSSVSYMHWYQQKPGKAPKRLMYDTSKLASGVPARFIGS
GSGTEFTLTISSLQPDDFATYYCQQWSSNPLTFGGGTKVEIK
SEQ ID NO: 116 (AA, HA CD3-VH)
QVQLVQSGAEVKKPGSSVKVSCKASGYKFTSYVMHWVKQAPGQGLEWIGYINPYNDVTKYNEKF
KGKATLTADESTNTAYMELSSLRSEDTAVHYCARGSYYDYDGFVYWGQGTLVTVSS
SEQ ID NO: 117 (AA, HA CD3-VL)
DIQMTQSPSTLSASVGDRVTMTCSATSSVSYMHWYQQKPGKAPKRWIYDTSKLASGVPARFIGS
GSGTEFTLTISSLQPDDFATYYCQQWSSNPLTFGGGTKVEIK
SEQ ID NO: 118 (AA, Troodon-66 CD3-VH)
QVQLQQSGAELARPGASVKMSCKTSGYTFTRYTMHWVKQRPGQGLEWIGYINPSRGYTNYNQKF
KDKATLITDKSSSTAYMQLSSLTSEDSAVYYCARYYDDHYSLDYWGQGTTLTVSS
SEQ ID NO: 119 (AA, Troodon-66 CD3-VL)
QIVLTQSPAIMSASPGEKVTMTCRASSSVSYMNWYQQKSGTSPKRWIYDTSKVASGVPYRFSGS
GSGTSYSLTISSMEAEDAATYYCQQWSSNPLTFGAGTKLELK
SEQ ID NO: 120 (nt, TEV 5′ UTR)
UCUCAACACAACAUAUACAAAACAAACGAAUCUCAAGCAAUCAAGCAUUCUACUUCUAUUGCAG
CAAUUUAAAUCAUUUCUUUUAAAGCAAAAGCAAUUUUCUGAAAAUUUUCACCAUUUACGAACGA
UAGC
SEQ ID NO: 121 (nt, FI 3′ UTR)
CUGGUACUGCAUGCACGCAAUGCUAGCUGCCCCUUUCCCGUCCUGGGUACCCCGAGUCUCCCCC
GACCUCGGGUCCCAGGUAUGCUCCCACCUCCACCUGCCCCACUCACCACCUCUGCUAGUUCCAG
ACACCUCCCAAGCACGCAGCAAUGCAGCUCAAAACGCUUAGCCUAGCCACACCCCCACGGGAAA
CAGCAGUGAUUAACCUUUAGCAAUAAACGAAAGUUUAACUAAGCUAUACUAACCCCAGGGUUGG
UCAAUUUCGUGCCAGCCACACC
SEQ ID NO: 122 (AA, anti-human serum albumin nanobody)
TRVDQTPRTATRETGESLTINCVLIDTSYPLYSTYWYRKNPGSSNKEQISISGRYVESVNKGTK
SFSLRIKDLIVADSATYICRAMGTNIWTGDGAGTVLTVN
SEQ ID NO: 123 (AA, rabbit anti-human serum albumin, heavy
chain)
EVQLLESGGGLVQPGGSLRLSCAVSGIDLSNYAINWVRQAPGKGLEWIGIIWASGTTFYATWAK
GRFTISRDNSKNTVYLQMNSLRAEDTAVYYCARTVPGYSTAPYFDLWGQGTLVTVSS
SEQ ID NO: 124 (AA, rabbit anti-human serum albumin, light
chain)
DIQMTQSPSSVSASVGDRVTIICQSSPSVWSNFLSWYQQKPGKAPKLLIYEASKLTSGVPSRFS
GSGSGTDFTLTISSLQPEDFATYYCGGGYSSISDTTFGGGTKVEIK
SEQ ID NO: 125 (AA, human IgG1 Fc region)
EPKSCDKTHTCPPCPAPELLGGPSVFLFPPKPKDTLMISRTPEVTCVVVDVSHEDPEVKENWYV
DGVEVHNAKTKPREEQYNSTYRVVSVLIVLHQDWLNGKEYKCKVSNKALPAPIEKTISKAKGQP
REPQVYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK
SEQ ID NO: 126 (nt, 5′ UTR)
AGACCCAAGCUGGCUAGCUCUAAAGAAGCCCCUGGGAGCACAGCUCAUCACC
SEQ ID NO: 127 (nt, 5′ UTR with Kozak sequence)
AGACCCAAGCUGGCUAGCUCUAAAGAAGCCCCUGGGAGCACAGCUCAUCACCGCCACC
SEQ ID NO: 128 (nt, 3′ UTR)
CUAUGAAGAAGGAAGGCAUCCAGACCAGAAACCGAAAAAUGUCUAGCAAAUCCAAAAAGUGCAA
AAAAGUGCAUGACUCACUGGAGGACUUCCCCAAGAACAGCUCGUUUAACCCGGCCGCCCUCUCC
AGACACAUGUCCUCCCUGAGCCACAUCUCGCCCUUCAGCCACUCCAGCCACAUGCUGACCACGC
CCACGCCGAUGCACCCGCCAUCCAGC
SEQ ID NO: 129 (AA, Pterodactyl-4)
MYRMQLLSCIALSLALVINSDIVLTQSPPSLAMSLGKRATISCRASESVTILGSHLIHWYQQKP
GQPPTLLIQLASNVQTGVPARFSGSGSRTDFTLTIDPVEEDDVAVYYCLQSRTIPRTFGGGTKL
EIKGSTSGSGKPGSGEGSTKGQIQLVQSGPELKKPGETVKISCKASGYTFTDYSINWVKRAPGK
GLKWMGWINTETREPAYAYDFRGRFAFSLETSASTAYLQINNLKYEDTATYFCALDYSYAMDYW
GQGTSVTVSSGGGGSQVQLQQSGAELARPGASVKMSCKTSGYTFTRYTMHWVKQRPGQGLEWIG
YINPSRGYTNYNQKFKDKATLITDKSSSTAYMQLSSLTSEDSAVYYCARYYDDHYSLDYWGQGT
TLTVSSGGGGSGGGGSGGGGSGGGGSQIVLTQSPAIMSASPGEKVIMTCRASSSVSYMNWYQQK
SGTSPKRWIYDTSKVASGVPYRFSGSGSGTSYSLTISSMEAEDAATYYCQQWSSNPLTFGAGTK
LELKSSGGSGGSGGSGGSGGSGGSGGSGGSGGSGSDIVLTQSPPSLAMSLGKRATISCRASESV
TILGSHLIHWYQQKPGQPPTLLIQLASNVQTGVPARFSGSGSRTDFTLTIDPVEEDDVAVYYCL
QSRTIPRTFGGGTKLEIKGSTSGSGKPGSGEGSTKGQIQLVQSGPELKKPGETVKISCKASGYT
FTDYSINWVKRAPGKGLKWMGWINTETREPAYAYDFRGRFAFSLETSASTAYLQINNLKYEDTA
TYFCALDYSYAMDYWGQGTSVTVSS
SEQ ID NO: 130 (AA, Pterodactyl-5)
MYRMQLLSCIALSLALVINSDIVLTQSPASLAVSPGQRATITCRASESVSFLGINLIHWYQQKP
GQPPKLLIYSASNLQSGVPARFSGSGSGTDFTLTISSVEPEDTANYYCLQSRTLPRTFGQGTKV
EIKGSTSGSGKPGSGEGSTKGQIQLVQSGPELKKPGGSVKISCKASGYTFTSYSINWVRQAPGK
GLEWVGWINTETREPAYAQGFTGRFTFSADTSKSMAYLQINSLRAEDTAVYYCALDYLYSLDFW
GQGTLVTVSSGGGGSQVQLVQSGAEVKKPGASVKVSCKASGYTFTRYTMHWVRQAPGQGLEWIG
YINPSKGYTNYNQKFKGRVTMTADKSTSTAYMELSSLRSEDTAVYYCARYYDDHYSLDYWGQGT
LVTVSSGGGGSGGGGSGGGGSGGGGSEIVMTQSPATLSLSPGERVTLSCRASSSVSYMNWYQQK
SGTSPKRWIYDTSKVASGVPYRFSGSGSGTDFTLTISSLQAEDEATYYCQQWSSNPLTFGQGTK
LEIKSSGGSGGSGGSGGSGGSGGSGGSGGSGGSGSDIVLTQSPASLAVSPGQRATITCRASESV
TILGSHLIHWYQQKPGQPPKLLINLASNVNTGVPARFSGSGSGTDFTLTISSVEPEDIANYYCL
QSRTLPRTFGQGTKVEIKGSTSGSGKPGSGEGSTKGQIQLVQSGPELKKPGGSVKISCKASGYT
FTDYSINWVRQAPGKGLEWVGWINTETREPAYAYDFTGRFTFSADTSKSMAYLQINSLRAEDTA
VYYCALDYTYGMDYWGQGTLVTVSS
SEQ ID NO: 131 (AA, Pterodactyl-6)
MYRMQLLSCIALSLALVINSDIVLTQSPPSLAMSLGKRATISCRASESVTILGSHLIHWYQQKP
GQPPTLLIQLASNVQTGVPARFSGSGSRTDFTLTIDPVEEDDVAVYYCLQSRTIPRTFGGGTKL
EIKGSTSGSGKPGSGEGSTKGQIQLVQSGPELKKPGETVKISCKASGYTFTDYSINWVKRAPGK
GLKWMGWINTETREPAYAYDERGRFAFSLETSASTAYLQINNLKYEDTATYFCALDYSYAMDYW
GQGTSVTVSSGGGGSQVQLVQSGAEVKKPGASVKVSCKASGYTFTRYTMHWVRQAPGQGLEWIG
YINPSKGYTNYNQKFKGRVTMTADKSTSTAYMELSSLRSEDTAVYYCARYYDDHYSLDYWGQGT
LVTVSSGGGGSGGGGSGGGGSGGGGSEIVMTQSPATLSLSPGERVTLSCRASSSVSYMNWYQQK
SGTSPKRWIYDTSKVASGVPYRFSGSGSGTDFTLTISSLQAEDEATYYCQQWSSNPLTFGQGTK
LEIKSSGGSGGSGGSGGSGGSGGSGGSGGSGGSGSDIVLTQSPPSLAMSLGKRATISCRASESV
TILGSHLIHWYQQKPGQPPTLLIQLASNVQTGVPARFSGSGSRTDFTLTIDPVEEDDVAVYYCL
QSRTIPRTFGGGTKLEIKGSTSGSGKPGSGEGSTKGQIQLVQSGPELKKPGETVKISCKASGYT
FTDYSINWVKRAPGKGLKWMGWINTETREPAYAYDERGRFAFSLETSASTAYLQINNLKYEDTA
TYFCALDYSYAMDYWGQGTSVTVSS
SEQ ID NO: 132 (AA, Pterodactyl-11)
MYRMQLLSCIALSLALVINSDIVLTQSPASLAVSPGQRATITCRASESVTILGSHLIHWYQQKP
GQPPKLLINLASNVNTGVPARFSGSGSGTDFTLTISSVEPEDTANYYCLQSRTLPRTFGQGTKV
EIKGSTSGSGKPGSGEGSTKGQIQLVQSGPELKKPGGSVKISCKASGYTFTDYSINWVRQAPGK
GLEWVGWINTETREPAYAYDFTGRFTFSADTSKSMAYLQINSLRAEDTAVYYCALDYTYGMDYW
GQGTLVTVSSGGGGSQVQLVQSGAEVKKPGASVKVSCKASGYTFTRYTMHWVRQAPGQGLEWMG
YINPSKGYTNYNQKFKGRVTITADKSTSTAYMELSSLRSEDTAVYYCARYYDDHYSLDYWGQGT
LVTVSSGGGGSGGGGSGGGGSGGGGSEIVMTQSPATLSVSPGERATLSCRASSSVSYLNWYQQK
PGTAPKRWIYDTSKVASGVPYRFSGSGSGIDFTLTISSLQAEDEATYYCQQWSSNPLTFGQGTK
LEIK
SEQ ID NO: 133 (AA, Pterodactyl-12)
MYRMQLLSCIALSLALVINSDIVLTQSPASLAVSPGQRATITCRASESVSFLGINLIHWYQQKP
GQPPKLLIYSASNLQSGVPARFSGSGSGTDFTLTISSVEPEDTANYYCLQSRTLPRTFGQGTKV
EIKGSTSGSGKPGSGEGSTKGQIQLVQSGPELKKPGGSVKISCKASGYTFTSYSINWVRQAPGK
GLEWVGWINTETREPAYAQGFTGRFTFSADTSKSMAYLQINSLRAEDTAVYYCALDYLYSLDFW
GQGTLVTVSSGGGGSQVQLVQSGAEVKKPGASVKVSCKASGYTFTRYTMHWVRQAPGQGLEWMG
YINPSKGYTNYNQKFKGRVTITADKSTSTAYMELSSLRSEDTAVYYCARYYDDHYSLDYWGQGT
LVTVSSGGGGSGGGGSGGGGSGGGGSEIVMTQSPATLSVSPGERATLSCRASSSVSYLNWYQQK
PGTAPKRWIYDISKVASGVPYRFSGSGSGTDFTLTISSLQAEDEATYYCQQWSSNPLTFGQGTK
LEIKSSGGSGGSGGSGGSGGSGGSGGSGGSGGSGSDIVLTQSPASLAVSPGQRATITCRASESV
TILGSHLIHWYQQKPGQPPKLLINLASNVNTGVPARFSGSGSGTDFTLTISSVEPEDTANYYCL
QSRTLPRTFGQGTKVEIKGSTSGSGKPGSGEGSTKGQIQLVQSGPELKKPGGSVKISCKASGYT
FTDYSINWVRQAPGKGLEWVGWINTETREPAYAYDFTGRFTFSADTSKSMAYLQINSLRAEDTA
VYYCALDYTYGMDYWGQGTLVTVSS
SEQ ID NO: 134 (AA, Pterodactyl-13)
MYRMQLLSCIALSLALVINSDIVLTQSPASLAVSPGQRATITCRASESVTILGSHLIHWYQQKP
GQPPKLLINLASNVNTGVPARFSGSGSGTDFTLTISSVEPEDTANYYCLQSRTLPRTFGQGTKV
EIKGSTSGSGKPGSGEGSTKGQIQLVQSGPELKKPGGSVKISCKASGYTFTDYSINWVRQAPGK
GLEWVGWINTETREPAYAYDFTGRFTFSADTSKSMAYLQINSIRAEDTAVYYCALDYTYGMDYW
GQGTLVTVSSGGGGSQVQLVQSGAEVKKPGASVKVSCKASGYTFTRYTMHWVRQAPGQGLEWMG
YINPSKGYTNYNQKFKGRVTITADKSTSTAYMELSSLRSEDTAVYYCARYYDDHYSLDYWGQGT
LVTVSSGGGGSGGGGSGGGGSGGGGSEIVMTQSPATLSVSPGERATLSCRASSSVSYLNWYQQK
PGTAPKRWIYDTSKVASGVPYRFSGSGSGTDFTLTISSLQAEDEATYYCQQWSSNPLTFGQGTK
LEIKSSGGSGGSGGSGGSGGSGGSGGSGGSGGSGSDIVLTQSPASLAVSPGQRATITCRASESV
TILGSHLIHWYQQKPGQPPKLLINLASNVNTGVPARFSGSGSGTDFTLTISSVEPEDTANYYCL
QSRTLPRTFGQGTKVEIKGSTSGSGKPGSGEGSTKGQIQLVQSGPELKKPGGSVKISCKASGYT
FTDYSINWVRQAPGKGLEWVGWINTETREPAYAYDFTGRFTFSADTSKSMAYLQINSLRAEDTA
VYYCALDYTYGMDYWGQGTLVTVSS
SEQ ID NO: 135 (AA, Pterodactyl-15)
MYRMQLLSCIALSLALVINSDIVLTQSPASLAVSPGQRATISCRASESVSFLGINLIHWYQQKP
GQPPKLLIYLASNLETGVPARFSGSGSGIDFTLTISSVEPEDTANYYCLQSRTLPRTFGQGTKV
EIKGSTSGSGKPGSGEGSTKGQIQLVQSGPELKKPGGSVKISCKASGYTFTSYSINWVRQAPGK
GLEWVGWINTETREPAYAQGFTGRFTFSADTSKSMAYLQINSLRAEDTAVYYCALDYLYSLDFW
GQGTLVTVSSGGGGSQVQLVQSGAEVKKPGASVKVSCKASGYTFTRYTMHWVRQAPGQGLEWMG
YINPSKGYTNYNQKFKGRVTITADKSTSTAYMELSSLRSEDIAVYYCARYYDDHYSLDYWGQGT
LVTVSSGGGGSGGGGSGGGGSGGGGSEIVMTQSPATLSVSPGERATLSCRASSSVSYLNWYQQK
PGTAPKRWIYDTSKVASGVPYRFSGSGSGTDFTLTISSLQAEDEATYYCQQWSSNPLTFGQGTK
LEIKSSGGSGGSGGSGGSGGSGGSGGSGGSGGSGSDIVLTQSPASLAVSPGQRATISCRASESV
SFLGINLIHWYQQKPGQPPKLLIYLASNLETGVPARFSGSGSGTDFTLTISSVEPEDTANYYCL
QSRTLPRTFGQGTKVEIKGSTSGSGKPGSGEGSTKGQIQLVQSGPELKKPGGSVKISCKASGYT
FTSYSINWVRQAPGKGLEWVGWINTETREPAYAQGFTGRFTFSADTSKSMAYLQINSLRAEDTA
VYYCALDYLYSLDFWGQGTLVTVSS
SEQ ID NO: 136 (AA, Pterodactyl-16)
MYRMQLLSCIALSLALVINSDIVLTQSPASLAVSPGQRATITCRASESVTILGSHLIHWYQQKP
GQPPKLLINLASNVNTGVPARFSGSGSGTDFTLTISSVEPEDTANYYCLQSRTLPRTFGQGTKV
EIKGSTSGSGKPGSGEGSTKGQIQLVQSGPELKKPGGSVKISCKASGYTFTDYSINWVRQAPGK
GLEWVGWINTETREPAYAYDFTGRFTFSADTSKSMAYLQINSLRAEDTAVYYCALDYTYGMDYW
GQGTLVTVSSGGGGSQVQLVQSGAEVKKPGASVKVSCKASGYTFTRYTMHWVRQAPGQGLEWIG
YINPSKGYTNYNQKFKGRVTMTADKSTSTAYMELSSLRSEDTAVYYCARYYDDHYSLDYWGQGT
LVTVSSGGGGSGGGGSGGGGSGGGGSEIVMTQSPATLSLSPGERVTLSCRASSSVSYMNWYQQK
SGTSPKRWIYDTSKVASGVPYRFSGSGSGTDFTLTISSLQAEDEATYYCQQWSSNPLTFGQGTK
LEIKSSGGSGGSGGSGGSGGSGGSGGSGGSGGSGSDIVLTQSPASLAVSPGQRATITCRASESV
TILGSHLIHWYQQKPGQPPKLLINLASNVNTGVPARFSGSGSGTDFTLTISSVEPEDTANYYCL
QSRTLPRTFGQGTKVEIKGSTSGSGKPGSGEGSTKGQIQLVQSGPELKKPGGSVKISCKASGYT
FTDYSINWVRQAPGKGLEWVGWINTETREPAYAYDFTGRFTFSADTSKSMAYLQINSLRAEDTA
VYYCALDYTYGMDYWGQGTLVTVSS
SEQ ID NO: 137 (AA, Pterodactyl-17)
MYRMQLLSCIALSLALVINSDIVLTQSPASLAVSPGQRATISCRASESVSFLGINLIHWYQQKP
GQPPKLLIYLASNLETGVPARFSGSGSGTDFTLTISSVEPEDTANYYCLQSRTLPRTFGQGTKV
EIKGSTSGSGKPGSGEGSTKGQIQLVQSGPELKKPGGSVKISCKASGYTFTSYSINWVRQAPGK
GLEWVGWINTETREPAYAQGFTGRFTFSADTSKSMAYLQINSLRAEDTAVYYCALDYLYSLDFW
GQGTLVTVSSGGGGSQVQLVQSGAEVKKPGASVKVSCKASGYTFTRYTMHWVRQAPGQGLEWIG
YINPSKGYTNYNQKFKGRVTMTADKSTSTAYMELSSLRSEDTAVYYCARYYDDHYSLDYWGQGT
LVTVSSGGGGSGGGGSGGGGSGGGGSEIVMTQSPATLSLSPGERVTLSCRASSSVSYMNWYQQK
SGTSPKRWIYDTSKVASGVPYRFSGSGSGTDFTLTISSLQAEDEATYYCQQWSSNPLTFGQGTK
LEIKSSGGSGGSGGSGGSGGSGGSGGSGGSGGSGSDIVLTQSPASLAVSPGQRATISCRASESV
SFLGINLIHWYQQKPGQPPKLLIYLASNLETGVPARFSGSGSGTDFTLTISSVEPEDTANYYCL
QSRILPRIFGQGTKVEIKGSTSGSGKPGSGEGSTKGQIQLVQSGPELKKPGGSVKISCKASGYT
FTSYSINWVRQAPGKGLEWVGWINTETREPAYAQGFTGRFTFSADTSKSMAYLQINSLRAEDTA
VYYCALDYLYSLDFWGQGTLVTVSS
SEQ ID NO: 138 (AA, Zephyrosaurus-299 bispecific antibody)
MYRMQLLSCIALSLALVINSDIVLTQSPASLAVSPGQRATISCRASESVSFLGINLIHWYQQKP
GQPPKLLIYLASNLETGVPARFSGSGSGIDFTLTISSVEPEDTANYYCLQSRTLPRTFGQGTKV
EIKGSTSGSGKPGSGEGSTKGQIQLVQSGPELKKPGGSVKISCKASGYTFTSYSINWVRQAPGK
GLEWVGWINTETREPAYAQGFTGRFTFSADTSKSMAYLQINSLRAEDTAVYYCALDYLYSLDFW
GQGTLVTVSSGGGGSQVQLVQSGAEVKKPGASVKVSCKASGYTFTRYTMHWVRQAPGQGLEWIG
YINPSKGYTNYNQKFKGRVTMTADKSTSTAYMELSSLRSEDTAVYYCARYYDDHYSLDYWGQGT
LVTVSSGGGGSGGGGSGGGGSGGGGSEIVMTQSPATLSLSPGERVILSCRASSSVSYMNWYQQK
SGTSPKRWIYDTSKVASGVPYRFSGSGSGTDFTLTISSLQAEDEATYYCQQWSSNPLTFGQGTK
LEIKSSGGSGGSGGSGGSGGSGGSGGSGGSGGSGSDIVLTQSPASLAVSPGQRATISCRASESV
SFIGINLIHWYQQKPGQPPKLLIYLASNLETGVPARFSGSGSGTDFTLTISSVEPEDTANYYCL
QSRTLPRTFGQGTKVEIKGSTSGSGKPGSGEGSTKGQIQLVQSGPELKKPGGSVKISCKASGYT
FTSYSINWVRQAPGKGLEWVGWINTETREPAYAQGFTGRFTFSADTSKSMAYLQINSLRAEDTA
VYYCALDYLYSLDFWGQGTLVTVSS
SEQ ID NO: 139 (AA, human IL-2 signal sequence)
MYRMQLLSCIALSLALVINS
SEQ ID NO: 140 (AA, 13′-17′ anti-BCMA scFv)
DIVLTQSPASLAVSPGQRATISCRASESVSFLGINLIHWYQQKPGQPPKLLIYLASNLETGVPA
RFSGSGSGTDFTLTISSVEPEDTANYYCLQSRTLPRTFGQGTKVEIKGSTSGSGKPGSGEGSTK
GQIQLVQSGPELKKPGGSVKISCKASGYTFTSYSINWVRQAPGKGLEWVGWINTETREPAYAQG
FTGRFTFSADTSKSMAYLQINSIRAEDTAVYYCALDYLYSLDFWGQGTLVTVSS
SEQ ID NO: 141 (AA, C-term Myc-Flag tag)
GGSLEQKLISEEDLAANDILDYKDDDDKV

In some embodiments, this disclosure contemplates any of the scFv sequences provided herein (e.g., SEQ ID NOs: 1-64) having 1, 2, or 3, but no more than 3 conservative amino acid substitution. Accordingly, in some embodiments, any of the scFv sequences provided herein may comprise 1, 2 or 3 conservative amino acid substitution. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Where an amino acid sequence is recited or referenced, the skilled person will understand that a corresponding polynucleotide sequence can be readily obtained or deduced that encodes the amino acid sequence. Generally, naturally occurring polynucleotide sequences (or fragments thereof) that encode a naturally occurring protein of interest (or fragment thereof) can be obtained from public sequence databases, e.g., GenBank.

### DETAILED DESCRIPTION OF CERTAIN EMBODIMENTS

### Anti-BCMA, Anti-CD3 Multispecific Antibodies, Such as Bispecific and Trispecific Antibodies

In one aspect, the invention provides a bispecific antibody that binds each of: BCMA and CD3. This bispecific antibody has the capacity to bind a T cell, e.g., a host T cell, to a BCMA+ cell, e.g., a host myeloma cell or a pathogenic antibody-producing plasma cell. This binding induces a cell-mediated immune response intended to kill the unwanted BCMA+ cell. In some embodiments, the anti-BCMA, anti-CD3 bispecific antibody described herein is bivalent, trivalent, or tetravalent.

In some embodiments, the bispecific antibody comprises:
a. an anti-CD3 binding antibody portion comprising a sequence selected from the group consisting of SEQ ID NOs: 2-38; and
b. an anti-BCMA binding antibody portion comprising a sequence selected from the group consisting of SEQ ID NOs: 39-64.

In some embodiments, the bispecific antibody can be a variant of that described above. For example, the bispecific antibody can include one or more linkers between the anti-CD3 binding antibody portion and the anti-BCMA binding antibody portion. In some embodiments, the one or more of the linkers (as identified herein) of one or more of the antibody portions can be substituted by another suitable linker, e.g., selected from among SEQ ID NOs: 65-68 and 70-73, and Sequence A.

In some embodiments, the bispecific antibody further comprises a linker that is between the anti-BCMA binding antibody portion and the anti-CD3 binding antibody portion. In some embodiments, the linker comprises a sequence selected from the group consisting of SEQ ID NOs: 65-68 and 70-73, and Sequence A. In some embodiments, the linker consists of a sequence selected from the group consisting of SEQ ID NOs: 65-68 and 70-73, and Sequence A.

In some embodiments, a linker that is within the anti-CD3 binding antibody portion, within the anti-BCMA binding antibody portion, or between two such antibody portions is substituted by a linker comprising a sequence selected from the group consisting of SEQ ID NOs: 65-68 and 70-73, and Sequence A. In some embodiments, the linker comprises the sequence of SEQ ID: NO 65. In some embodiments, the linker comprises the sequence of SEQ ID NO: 66. In some embodiments, the linker comprises the sequence of SEQ ID NO: 67. In some embodiments, the linker comprises the sequence of SEQ ID NO: 68. In some embodiments, the linker comprises the sequence of Sequence A. In some embodiments, the linker comprises the sequence of SEQ ID NO: 70. In some embodiments, the linker comprises the sequence of SEQ ID NO: 71. In some embodiments, the linker comprises the sequence of SEQ ID NO: 72. In some embodiments, the linker comprises the sequence of SEQ ID NO: 73. In some embodiments, the linker connects a light chain and heavy chain. In some embodiments that linker connects two or more separate antibody portions.

In some embodiments, a linker that is within the anti-CD3 binding antibody portion, within the anti-BCMA binding antibody portion, or between two such antibody portions is substituted by a linker that is a full or partial sequence of a serum albumin protein. In some such embodiments, the serum albumin protein is human serum albumin. In some such embodiments, the linker comprises a sequence of SEQ ID NO: 74. In some such embodiments, the linker comprises a portion of the sequence of SEQ ID NO: 74, wherein that portion comprises at least 300 consecutive amino acids of that sequence. In some such embodiments, the linker consists of a sequence of SEQ ID NO: 74. In some embodiments, the linker connects a light chain and heavy chain. In some embodiments that linker connects two or more separate antibody portions.

In some embodiments of the multispecific antibody, such as a bispecific antibody, the anti-BCMA binding antibody portion or portions is located N-terminal with respect to the anti-CD3 binding antibody portion or portions. In some embodiments of the multispecific antibody, such as a bispecific antibody, the anti-CD3 binding antibody portion is located N-terminal with respect to the anti-BCMA binding antibody portion.

In some embodiments, the bispecific antibody comprises: an anti-BCMA antibody portion comprising:
a. an anti-CD3 binding antibody portion comprising a sequence selected from the group consisting of SEQ ID NOs: 2-38; and
b. an anti-BCMA binding antibody portion known in the art.

In some embodiments, the bispecific antibody is a variant of that described above. For example, the bispecific antibody can include one or more linkers between the anti-CD3 binding antibody portion and the anti-BCMA binding antibody portion. In some embodiments, the one or more of the linkers (as identified herein) of one or more of the antibody portions is substituted by another suitable linker, e.g., selected from among SEQ ID NOs: 65-68 and 70-73, and Sequence A.

In some embodiments, the bispecific antibody comprises: an anti-BCMA antibody portion comprising:
a. an anti-CD3 binding antibody portion known in the art; and
b. an anti-BCMA binding antibody portion comprising a sequence selected from the group consisting of SEQ ID NOs: 39-64.

In some embodiments, the bispecific antibody is a variant of that described above. For example, the bispecific antibody can include one or more linkers between the anti-CD3 binding antibody portion and the anti-BCMA binding antibody portion. In some embodiments, the one or more of the linkers (as identified herein) of one or more of the antibody portions is substituted by another suitable linker, e.g., selected from among SEQ ID NOs: 65-68 and 70-73, and Sequence A.

In some embodiments, wherein the bispecific antibody is bivalent, it comprises exactly one anti-CD3 binding antibody portion comprising a sequence selected from the group consisting of SEQ ID NOs: 2-38. In some embodiments, wherein the bispecific antibody is bivalent, it comprises exactly one anti-BCMA binding antibody portion comprising a sequence selected from the group consisting of SEQ ID NOs: 39-64. In some embodiments, wherein the bispecific antibody is bivalent, it comprises exactly one anti-CD3 binding antibody portion comprising a sequence selected from the group consisting of SEQ ID NOs: 2-38, and exactly one anti-BCMA binding antibody portion comprising a sequence selected from the group consisting of SEQ ID NOs: 39-64. In some embodiments, wherein the bispecific antibody is bivalent, it comprises exactly one anti-CD3 binding antibody portion comprising a sequence selected from the group consisting of SEQ ID NOs: 2-38 and exactly one anti-BCMA binding antibody portion known in the art. In some embodiments, wherein the bispecific antibody is bivalent, it comprises exactly one anti-CD3 binding antibody portion known in the art and exactly one anti-BCMA binding antibody portion comprising a sequence selected from the group consisting of SEQ ID NOs: 39-64. In any of the foregoing embodiments of this paragraph, the bispecific antibody can further comprise a linker that is between the anti-BCMA binding antibody portion and the anti-CD3 binding antibody portion. In some embodiments, the linker comprises an amino acid sequence selected from the group consisting of SEQ ID NOs: 65-68 and 70-73, and Sequence A. In some embodiments, the linker consists of an amino acid sequence selected from the group consisting of SEQ ID NOs: 65-68 and 70-73, and Sequence A. In some embodiments, the linker comprises a full or partial sequence of a serum albumin protein. In some such embodiments, the linker comprises a sequence of SEQ ID NO: 74. In some such embodiments, the linker comprises a portion of the sequence of SEQ ID NO: 74, wherein that portion comprises at least 300 consecutive amino acids of that sequence. In some such embodiments, the linker consists of a sequence of SEQ ID NO: 74.

In some embodiments, the linker may be substituted by any other suitable linker. For the purposes of the invention, the linkers of SEQ ID NOs: 65-68 and 70-73, and Sequence A, are considered interchangeable.

In some embodiments, wherein the bispecific antibody is trivalent, it comprises exactly two anti-CD3 binding antibody portions, each comprising a sequence selected from the group consisting of SEQ ID NOs: 2-38. In some embodiments, wherein the bispecific antibody is trivalent, it comprises exactly two anti-BCMA binding antibody portions, each comprising a sequence selected from the group consisting of SEQ ID NOs: 39-64. In some embodiments, wherein the bispecific antibody is trivalent, it comprises exactly one anti-CD3 binding antibody portion comprising a sequence selected from the group consisting of SEQ ID NOs: 2-38 and exactly two anti-BCMA binding antibody portions, each comprising a sequence selected from the group consisting of SEQ ID NOs: 39-64. In some embodiments, wherein the bispecific antibody is trivalent, it comprises exactly two anti-CD3 binding antibody portions, each comprising a sequence selected from the group consisting of SEQ ID NOs: 2-38, and exactly one anti-BCMA binding antibody portion comprising a sequence selected from the group consisting of SEQ ID NOs: 39-64. In some embodiments, wherein the bispecific antibody is trivalent, it comprises exactly one anti-CD3 binding antibody portion comprising a sequence selected from the group consisting of SEQ ID NOs: 2-38. In some embodiments, wherein the bispecific antibody is trivalent, it comprises exactly one anti-BCMA binding antibody portions comprising a sequence selected from the group consisting of SEQ ID NOs: 39-64. In some embodiments, wherein the bispecific antibody is trivalent, it comprises exactly one anti-CD3 binding antibody portion comprising a sequence selected from the group consisting of SEQ ID NOs: 2-38 and exactly one anti-BCMA binding antibody portion comprising a sequence selected from the group consisting of SEQ ID NOs: 39-64. In any of the foregoing embodiments, where two sequences are simultaneously selected from the same Markush group, the two sequences can be the same or different. In any of the foregoing embodiments of this paragraph, where one or more anti-BCMA or anti-CD3 binding antibody portions is not identified by reference to a specific sequence, that anti-BCMA or anti-CD3 binding antibody portion can be an antibody portion known in the art. In any of the foregoing embodiments of this paragraph, the bispecific antibody can further comprise one or two linkers, wherein each linker connects two binding antibody portions. In some embodiments, the linkers comprise an amino acid sequence selected from the group consisting of SEQ ID NOs: 65-68 and 70-73, and Sequence A. In some embodiments, the linkers consist of an amino acid sequence selected from the group consisting of SEQ ID NOS: 65-68 and 70-74, and Sequence A. In some embodiments, the linkers comprise a full or partial sequence of a serum albumin protein. In some such embodiments, the serum albumin protein is human serum albumin.

In some embodiments, wherein the bispecific antibody is tetravalent, it comprises exactly two anti-CD3 binding antibody portions, each comprising a sequence selected from the group consisting of SEQ ID NOs: 2-38. In some embodiments, wherein the bispecific antibody is tetravalent, it comprises exactly two anti-BCMA binding antibody portions, each comprising a sequence selected from the group consisting of SEQ ID NOs: 39-64. In some embodiments, wherein the bispecific antibody is tetravalent, it comprises exactly two anti-CD3 binding antibody portions, each comprising a sequence selected from the group consisting of SEQ ID NOs: 2-38, and exactly two anti-BCMA binding antibody portions, each comprising a sequence selected from the group consisting of SEQ ID NOs: 39-64. In some embodiments, wherein the bispecific antibody is tetravalent, it comprises exactly one anti-CD3 binding antibody portion comprising a sequence selected from the group consisting of SEQ ID NOs: 2-38 and exactly two anti-BCMA binding antibody portions, each comprising a sequence selected from the group consisting of SEQ ID NOs: 39-64. In some embodiments, wherein the bispecific antibody is tetravalent, it comprises exactly two anti-CD3 binding antibody portions, each comprising a sequence selected from the group consisting of SEQ ID NOs: 2-38, and exactly one anti-BCMA binding antibody portion comprising a sequence selected from the group consisting of SEQ ID NOs: 39-64. In some embodiments, wherein the bispecific antibody is tetravalent, it comprises exactly one anti-CD3 binding antibody portion comprising a sequence selected from the group consisting of SEQ ID NOs: 2-38. In some embodiments, wherein the bispecific antibody is tetravalent, it comprises exactly one anti-BCMA binding antibody portions comprising a sequence selected from the group consisting of SEQ ID NOs: 39-64. In some embodiments, wherein the bispecific antibody is tetravalent, it comprises exactly one anti-CD3 binding antibody portion comprising a sequence selected from the group consisting of SEQ ID NOs: 2-38 and exactly one anti-BCMA binding antibody portion comprising a sequence selected from the group consisting of SEQ ID NOs: 39-64. In any of the foregoing embodiments, where two sequences are simultaneously selected from the same Markush group, the two sequences can be the same or different. In any of the foregoing embodiments of this paragraph, where one or more anti-BCMA or anti-CD3 binding antibody portions is not identified by reference to a specific sequence, that anti-BCMA or anti-CD3 binding antibody portion can be an antibody portion known in the art. In any of the foregoing embodiments of this paragraph, the bispecific antibody can further comprise one or more linkers, wherein each linker connects two binding antibody portions. In some embodiments, the linkers comprise an amino acid sequence selected from the group consisting of SEQ ID NOs: 65-68 and 70-73, and Sequence A. In some embodiments, the linkers consist of an amino acid sequence selected from the group consisting of SEQ ID NOs: 65-68 and 70-73, Sequence A. In some embodiments, the linkers comprise a full or partial sequence of a serum albumin protein. In some such embodiments, the serum albumin protein is human serum albumin. In some such embodiments, the linker comprises a sequence of SEQ ID NO: 74. In some such embodiments, the linker comprises a portion of the sequence of SEQ ID NO: 74, wherein that portion comprises at least 300 consecutive amino acids of that sequence. In some such embodiments, the linker consists of a sequence of SEQ ID NO: 74.

In any of the embodiments, a linker may generally be substituted by any other suitable linker. For the purposes of the invention, the linkers of SEQ ID NOs: 65-68 and 70-73, Sequence A, are considered interchangeable.

In some embodiments, provided is an anti-BCMA, anti-CD3 bispecific antibody comprising an amino acid sequence selected from the group consisting of SEQ ID NOs: 75-101. In some embodiments, provided is an anti-BCMA, anti-CD3 bispecific antibody consisting of an amino acid sequence selected from the group consisting of SEQ ID NOs: 75-101.

In some embodiments, provided is a bivalent anti-BCMA, anti-CD3 bispecific antibody comprising an amino acid sequence selected from the group consisting of SEQ ID NOs: 75-87. In some embodiments, provided is a bivalent anti-BCMA, anti-CD3 bispecific antibody consisting of an amino acid sequence selected from the group consisting of SEQ ID NOs: 75-87. In some embodiments, the antibody comprises the sequence of SEQ ID NO: 75. In some embodiments, the antibody comprises the sequence of SEQ ID NO: 76. In some embodiments, the antibody comprises the sequence of SEQ ID NO: 77. In some embodiments, the antibody comprises the sequence of SEQ ID NO: 78. In some embodiments, the antibody comprises the sequence of SEQ ID NO: 79. In some embodiments, the antibody comprises the sequence of SEQ ID NO: 80. In some embodiments, the antibody comprises the sequence of SEQ ID NO: 81. In some embodiments, the antibody comprises the sequence of SEQ ID NO: 82. In some embodiments, the antibody comprises the sequence of SEQ ID NO: 83. In some embodiments, the antibody comprises the sequence of SEQ ID NO: 84. In some embodiments, the antibody comprises the sequence of SEQ ID NO: 85. In some embodiments, the antibody comprises the sequence of SEQ ID NO: 86. In some embodiments, the antibody comprises the sequence of SEQ ID NO: 87. In some embodiments, variants or homologues of the bivalent anti-BCMA, anti-CD3 bispecific antibodies are provided which are at least about 70% identical, at least about 80% identical, at least about 90% identical, at least about 95% identical, at least about 98% identical, at least about 99% identical, at least about 99.5% identical, or at least about 99.9% to SEQ ID NOs: 75-87.

In any of the embodiments, a linker may generally be substituted by any other suitable linker. For the purposes of the invention, the linkers of SEQ ID NOs: 65-68 and 70-73, Sequence A, are considered interchangeable.

In some embodiments, provided is a trivalent anti-BCMA, anti-CD3 bispecific antibody comprising an amino acid sequence selected from the group consisting of SEQ ID NOs: 88-101. In some embodiments, provided is a bivalent anti-BCMA, anti-CD3 bispecific antibody consisting of an amino acid sequence selected from the group consisting of SEQ ID NOs: 88-101. In some embodiments, the antibody comprises the sequence of SEQ ID NO: 88. In some embodiments, the antibody comprises the sequence of SEQ ID NO: 89. In some embodiments, the antibody comprises the sequence of SEQ ID NO: 90. In some embodiments, the antibody comprises the sequence of SEQ ID NO: 91. In some embodiments, the antibody comprises the sequence of SEQ ID NO: 92. In some embodiments, the antibody comprises the sequence of SEQ ID NO: 93. In some embodiments, the antibody comprises the sequence of SEQ ID NO: 94. In some embodiments, the antibody comprises the sequence of SEQ ID NO: 95. In some embodiments, the antibody comprises the sequence of SEQ ID NO: 96. In some embodiments, the antibody comprises the sequence of SEQ ID NO: 97. In some embodiments, the antibody comprises the sequence of SEQ ID NO: 98. In some embodiments, the antibody comprises the sequence of SEQ ID NO: 99. In some embodiments, the antibody comprises the sequence of SEQ ID NO: 100. In some embodiments, the antibody comprises the sequence of SEQ ID NO: 101. In some embodiments, variants or homologues of the trivalent anti-BCMA, anti-CD3 bispecific antibodies are provided which are at least about 70% identical, at least about 80% identical, at least about 90% identical, at least about 95% identical, at least about 98% identical, at least about 99% identical, at least about 99.5% identical, or at least about 99.9% to SEQ ID NOs: 88-101.

In any of the embodiments, a linker may generally be substituted by any other suitable linker. For the purposes of the invention, the linkers of SEQ ID NOs: 65-68 and 70-73, Sequence A, are considered interchangeable.

The invention also provides trispecific antibodies that bind BCMA, CD3, and a third antigen. Thus, in some embodiments, provided is a trispecific antibody comprising an anti-BCMA binding antibody portion, an anti-CD3-binding antibody portion, and an antibody portion that binds a third antigen that is not BCMA or CD3. In some embodiments, the third antigen is serum albumin (SA); in some such embodiments, the third antibody portion is an anti-SA binding antibody portion; and in some such embodiments, the third antibody portion comprises the amino acid sequence of SEQ ID NO: 122, SEQ ID NO: 123, SEQ ID NO: 124, or both SEQ ID NO: 123 and SEQ ID NO: 124. In some embodiments, the third antigen is a cell surface protein, for example, an antigen selected from the group consisting of CD38, CD138, CD19, CD20, CD229, CD56, NKG2D, 2B4, NKp46, NKp44, CD14, CCR2, CCR5, FcRN, and CX3CR1. In some embodiments, the third antigen is selected from the group consisting of CD38, CD138, CD19, CD20, and CD229. In some embodiments, the third antigen is selected from the group consisting of CD56, NKG2D, 2B4, NKp46, and NKp44. In some embodiments, the third antigen is selected from the group consisting of CD14, CCR2, CCR5, and CX3CR1. In some embodiments, the third antigen is CD38. In some embodiments, the third antigen is CD138. In some embodiments, the third antigen is CD19. In some embodiments, the third antigen is CD20. In some embodiments, the third antigen is CD229. In some embodiments, the third antigen is CD56. In some embodiments, the third antigen is NKG2D. In some embodiments, the third antigen is 2B4. In some embodiments, the third antigen is NKp46. In some embodiments, the third antigen is NKp44. In some embodiments, the third antigen is CD14. In some embodiments, the third antigen is CCR2. In some embodiments, the third antigen is CCR5. In some embodiments, the third antigen is CX3CR1. Sequences of, or comprising, these antigens are available, by animal species and where applicable by subtype, in publicly accessible databases such as UniProt/SwissProt. The anti-CD3 binding antibody portions can comprise a sequence selected from the group consisting of SEQ ID: 2-38. The anti-BCMA binding antibody portion can comprise a sequence selected from the group consisting of SEQ ID NOs: 39-64. In some embodiments, the trispecific antibody comprises an anti-CD3 binding antibody portion comprising a sequence selected from the group consisting of SEQ ID NOs: 2-38, and an anti-BCMA binding antibody portion comprising a sequence selected from the group consisting of SEQ ID: NOs 39-64. In any of the foregoing embodiments of this paragraph, the trispecific antibody can further comprise one or more linkers between binding antibody portions. In some embodiments, the linker(s) comprises an amino acid sequence selected from the group consisting of SEQ ID NOs: 65-68 and 70-73, Sequence A. In some embodiments, the linker(s) consists of an amino acid sequence selected from the group consisting of SEQ ID NOs: 65-68 and 70-73, Sequence A. In some embodiments, the linker(s) comprises an amino acid sequence selected from the group consisting of SEQ ID: 88. In some embodiments, the linkers comprise a full or partial sequence of a serum albumin protein, provided that in some cases, inclusion of anti-HSA antibody portion can be incompatible with inclusion of an HSA linker. In some such embodiments, the serum albumin protein is human serum albumin. In some such embodiments, the linker comprises a sequence of SEQ ID NO: 74. In some such embodiments, the linker comprises a portion of the sequence of SEQ ID NO: 74, wherein that portion comprises at least 300 consecutive amino acids of that sequence. In some such embodiments, the linker consists of a sequence of SEQ ID NO: 74.

In any of the embodiments, a linker may generally be substituted by any other suitable linker. For the purposes of the invention, the linkers of SEQ ID NOs: 65-68 and 70-73, Sequence A, are considered interchangeable. In any instance where two or more linkers in a protein are simultaneously substituted, the linker to be used may be independently selected from the linkers of SEQ ID NOs: 65-68 and 70-73, Sequence A.

### Novel Anti-BCMA Binding Antibody Portions

In another aspect, the invention also provides a protein comprising an anti-BCMA binding antibody portion comprising the sequence of SEQ ID NO: 41. The invention also provides a protein comprising an anti-BCMA binding antibody portion comprising a variant of SEQ ID NO: 41, wherein the linker GSTSGSGKPGSGEGSTKG (SEQ ID NO: 71) is substituted with another suitable linker, e.g., a linker of any of SEQ ID NOs: 65-73. For example, in some embodiments, the linker GSTSGSGKPGSGEGSTKG (SEQ ID NO: 71) is substituted with the linker of SEQ ID NO: 65. In some embodiments, the linker GSTSGSGKPGSGEGSTKG (SEQ ID NO: 71) is substituted with the linker of SEQ ID NO: 66. In some embodiments, the linker GSTSGSGKPGSGEGSTKG (SEQ ID NO: 71) is substituted with the linker of SEQ ID NO: 67. In some embodiments, the linker GSTSGSGKPGSGEGSTKG (SEQ ID NO: 71) is substituted with the linker of SEQ ID NO: 68. In some embodiments, the linker

GSTSGSGKPGSGEGSTKG (SEQ ID NO: 71) is substituted with the linker of Sequence A. In some embodiments, the linker GSTSGSGKPGSGEGSTKG (SEQ ID NO: 71) is substituted with the linker of SEQ ID NO: 70. In some embodiments, the linker GSTSGSGKPGSGEGSTKG (SEQ ID NO: 71) is the linker of SEQ ID NO: 71. In some embodiments, the linker GSTSGSGKPGSGEGSTKG (SEQ ID NO: 71) is substituted with the linker of SEQ ID NO: 72. In some embodiments, the linker GSTSGSGKPGSGEGSTKG (SEQ ID NO: 71) is substituted with the linker of SEQ ID NO: 73. In some embodiments, the protein is an antibody, e.g., a therapeutic monoclonal antibody. In some embodiments, the protein is a bispecific or multispecific antibody. In some embodiments, the protein is a chimeric antigen receptor further comprising a transmembrane domain and a cytoplasmic domain; the selection and construction of such domains is known in the art.

### Novel Anti-CD3 Binding Antibody Portions

In one aspect, the invention provides a protein comprising an anti-CD3 binding antibody portion comprising a sequence selected from the group consisting of SEQ ID NOs: 2-38. The invention also provides a protein comprising an anti-CD3 binding antibody portion comprising a variant of any of SEQ ID NOs: 2-38, wherein the linker, e.g., GGGGSGGGGSGGGGSGGGGS (SEQ ID NO: 68), is substituted with another suitable linker, e.g., a linker of any of SEQ ID NOs: 65-73. For example, in some embodiments, the linker is the linker of SEQ ID NO: 65. For example, in some embodiments, the linker is the linker of SEQ ID NO: 66. For example, in some embodiments, the linker is the linker of SEQ ID NO: 67. For example, in some embodiments, the linker is the linker of SEQ ID NO: 68. For example, in some embodiments, the linker is the linker of Sequence A. For example, in some embodiments, the linker is the linker of SEQ ID NO: 70. For example, in some embodiments, the linker is the linker of SEQ ID NO: 71. For example, in some embodiments, the linker is the linker of SEQ ID NO: 72. For example, in some embodiments, the linker is the linker of SEQ ID NO: 73. In some embodiments, the protein is an antibody, e.g., a therapeutic monoclonal antibody. In some embodiments, the protein is a bispecific or multispecific antibody.

### Nucleic Acid Constructs

In some embodiments, the present invention encompasses a nucleic acid molecule (e.g., DNA or RNA) that encodes any of the novel proteins or protein fragments described herein, e.g., one or more proteins or protein fragments comprising, or consisting of, a sequence selected from the group consisting of SEQ ID NOs: 2-68, 70-105, and 110-138, Sequence A.

In some embodiments, the nucleic acid molecule is a DNA.

In some embodiments, the nucleic acid molecular is an mRNA construct suitable for expression in, transfection into, or a modification of, a cell, wherein the mRNA construct encodes one or more amino acid sequences (or sequence fragments) selected from the group consisting of SEQ ID NOs: 2-68, 70-105, and 110-138, Sequence A. In some embodiments, the mRNA construct encodes an anti-BCMA, anti-CD3 bispecific antibody comprising a sequence selected from the group consisting of SEQ ID NOs: 89-115.

One method for generating mRNA for use in transfection involves in vitro transcription (IVT) of a template with specially designed primers. Optionally, the mRNA is 3′ polyadenylated by methods known in the art and may comprise, for example, a 3′ polyadenine tail of about 25, 50, 100, 150, 250, 500, or 1000 adenine nucleotides.

In some embodiments, the nucleic acid is a self-amplifying RNA (saRNA). In some embodiments, the RNA, e.g., mRNA, comprises pseudouridine. In some embodiments, the RNA is artificially enriched in pseudouridine. In some embodiments, substantially all the uridine nucleotides (e.g., greater than 90%, 95%, 97%, 99% or 99.9%) of the RNA are substituted with pseudouridine. Methods for incorporating pseudouridine into an RNA are known in the art.

Generally, the mRNA construct will include 5′ and 3′ untranslated regions (“UTRs”). Such UTRs are known in the art and include, for example, those UTRs that occur naturally in IgG, alpha-fetoprotein, or actin genes. Exemplary UTRs for use in the current invention are provided (as RNA sequences) in SEQ ID NOs: 126, 127 and 128.

In some embodiments, the mRNA construct can encode more than one open reading frame and can optionally include an Internal Ribosome Entry Site (IRES).

### Engineered Cells

Another aspect, the invention provides cells, e.g., mammalian cells, e.g., human cells, modified to express one or more of the inventive multispecific antibodies, such as anti-BCMA, anti-CD3 bispecific antibodies, disclosed herein. In some embodiments, the cells are modified to secrete one or more of the inventive anti-BCMA, anti-CD3 bispecific antibodies disclosed herein. In some embodiments, the cells are further modified to express and/or secrete one or more other proteins, e.g., IL-12 and/or CXCR4. While some such proteins are secreted, others (e.g., CXCR4) are membrane bound, and others are intracellular.

### Methods to Obtain and Modify Cells

In some embodiments, the cells to be engineered are homospecific, i.e., obtained from a member of the same species for which administration of the modified cells is ultimately intended. Thus, cells are obtained from a human if they are to be modified for subsequent administration to a human. In some embodiments, the cells are derived from an individual different than the intended recipient of the modified cells (i.e., clinical heterologous use). In some other embodiments, the cells are derived from the same individual for whom they are intended once modified (i.e., autologous use). Homospecific cells can be obtained by methods known in the art and from several sources, including peripheral blood mononuclear cells, bone marrow, adipose tissue, lymph node tissue, umbilical cord, cord blood, thymus tissue, tissue from a site of infection, ascites, pleural effusion, spleen tissue, and tumors. Homospecific cells, including stem cells, may be generated from induced pluripotent stem cells or hematopoietic stem cells or progenitor cells. In some embodiments, any cell line available in the art, including but not limited to, stem cells lines, may be used.

In some embodiments, MSCs (mesenchymal stromal cells) for use in the present invention are isolated from bone marrow mononuclear cells, which can be obtained, for example, by bone marrow aspiration; umbilical cord tissue; adipose tissue; and/or a tooth or teeth.

Biological methods for introducing a polynucleotide of interest into a host cell include the use of DNA and RNA vectors. Viral vectors, and especially retroviral vectors, are a widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362, incorporated herein by reference.

Polynucleotides, e.g., exogenous polynucleotides, e.g., exogenous RNA or exogenous DNA, can be introduced into target cells by commercially available methods which include, but are not limited to: electroporation (AMAXA® NUCLEOFECTOR-II® (AMAXA Biosystems, Cologne, Germany)), (ECM 830 (BTX) (Harvard Instruments, Boston, Mass.) or the Gene Pulser II® (BioRad, Denver, Colo.), MULTIPORATOR® (Eppendorf, Hamburg Germany), mechanical membrane disruption (e.g., cell squeezing, see U.S. Pat. Pub. No. 2014/287509A1), cationic liposome mediated transfection using lipofection, polymer encapsulation, peptide mediated transfection, or biolistic particle delivery systems such as “gene guns” (scc, for example, Nishikawa, et al., Hum Gene Ther., 12 (8): 861-70 (2001).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipid formulations can be used to introduce nucleic acids into a host cell (in vitro, ex vivo, or in vivo). In another aspect, any novel nucleic acid described herein may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

### Engineered Cells Expressing an Anti-BCMA, Anti-CD3 Bispecific Antibody

In some embodiments, provided is a cell engineered to express an anti-BCMA, anti-CD3 bispecific antibody wherein the bispecific antibody comprises a sequence selected from the group consisting of SEQ ID NOs 2-38. In some embodiments, provided is a cell comprising an mRNA construct that encodes an amino acid sequence selected from the group consisting of SEQ ID NOs 2-38. In some embodiments, the mRNA construct is an exogenous RNA. In some embodiments, provided is a cell comprising a DNA construct that encodes an amino acid sequence selected from the group consisting of SEQ ID NOs 2-38. In some embodiments, the DNA construct is an exogenous DNA. In some embodiments, provided is a cell comprising a nucleic acid vector that encodes an amino acid sequence selected from the group consisting of SEQ ID NOs 2-38. In some embodiments, the cell is engineered to express a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 2-38.

In some embodiments, provided is a cell engineered to express an anti-BCMA, anti-CD3 bispecific antibody wherein the bispecific antibody comprises a sequence selected from the group consisting of SEQ ID NOs 39-64. In some embodiments, provided is a cell comprising an mRNA construct that encodes an amino acid sequence selected from the group consisting of SEQ ID NOs 39-64. In some embodiments, provided is a cell comprising a DNA construct that encodes an amino acid sequence selected from the group consisting of SEQ ID NOs 39-64. In some embodiments, provided is a cell comprising a nucleic acid vector that encodes an amino acid sequence selected from the group consisting of SEQ ID NOs 39-64. In some embodiments, the cell is engineered to express a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 39-64.

In some embodiments, provided is a cell engineered to express an anti-BCMA, anti-CD3 bispecific antibody wherein the bispecific antibody comprises a sequence selected from the group consisting of SEQ ID NOs 75-101. In some embodiments, provided is a cell comprising an mRNA construct that encodes an amino acid sequence selected from the group consisting of SEQ ID NOs 75-101. In some embodiments, provided is a cell comprising a DNA construct that encodes an amino acid sequence selected from the group consisting of SEQ ID NOs 75-101. In some embodiments, provided is a cell comprising a nucleic acid vector that encodes an amino acid sequence selected from the group consisting of SEQ ID NOs 75-101. In some embodiments, the cell is engineered to express a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 75-101.

In some embodiments, provided is a cell engineered to express an anti-BCMA, anti-CD3 bispecific antibody wherein the bispecific antibody comprises a sequence selected from the group consisting of SEQ ID NOs 75-87. In some embodiments, provided is a cell comprising an mRNA construct that encodes an amino acid sequence selected from the group consisting of SEQ ID NOs 75-87. In some embodiments, provided is a cell comprising a DNA construct that encodes an amino acid sequence selected from the group consisting of SEQ ID NOs 75-87. In some embodiments, provided is a cell comprising a nucleic acid vector that encodes an amino acid sequence selected from the group consisting of SEQ ID NOs 75-87. In some embodiments, the cell is engineered to express a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 75-87.

In some embodiments, provided is a cell engineered to express an anti-BCMA, anti-CD3 bispecific antibody wherein the bispecific antibody comprises a sequence selected from the group consisting of SEQ ID NOs 88-101. In some embodiments, provided is a cell comprising an mRNA construct that encodes an amino acid sequence selected from the group consisting of SEQ ID NOs 88-101. In some embodiments, provided is a cell comprising a DNA construct that encodes an amino acid sequence selected from the group consisting of SEQ ID NOs 88-101. In some embodiments, provided is a cell comprising a nucleic acid vector that encodes an amino acid sequence selected from the group consisting of SEQ ID NOs 88-101. In some embodiments, the cell is engineered to express a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 88-101.

In some embodiments, provided is a cell engineered to express an anti-BCMA, anti-CD3 bispecific antibody wherein the bispecific antibody comprises a sequence selected from the group consisting of SEQ ID NOs 88-101.

In some embodiments, provided is a cell comprising an mRNA construct that encodes an amino acid sequence selected from the group consisting of SEQ ID NOs 39-63 and 75-101.

In some embodiments, provided is a cell comprising an mRNA construct that encodes an amino acid sequence that is variant of a sequence selected from the group consisting of SEQ ID NOs 39-64 and 75-101, wherein each linker in such sequence is optionally substituted with a linker selected from the group consisting of SEQ ID NOs: 65-68 and 70-73, Sequence A.

### Engineered Cells Expressing an Anti-BCMA, Anti-CD3 Bispecific Antibody and IL-12

In some embodiments, the cell modified to express an anti-BCMA, anti-CD3 bispecific antibody is further modified to express IL-12. In its natural state, IL-12 consists of two subunits exemplified by the human amino acid sequences of SEQ ID NO: 103 (alpha subunit) and SEQ ID NO: 104 (beta subunit). In some embodiments, the cell is modified to secrete IL-12 by co-expression of its separate alpha and beta subunits. In some embodiments, the cell is modified to secrete IL-12 by expression of a fusion protein that comprises both the alpha (p35) and beta (p40) subunits of IL-12, optionally connected by a linker, e.g., a linker of any of SEQ ID NOs: 79-87. An example of one such engineered protein, suitable for use in the present invention, is a protein of SEQ ID NO: 105.

Thus, in some embodiments, the cell is modified to express: an anti-BCMA, anti-CD3 bispecific antibody comprising a sequence selected from the group consisting of SEQ ID NOs: 39-63 and 75-101; and IL-12. In some embodiments, the cell is modified to express: an anti-BCMA, anti-CD3 bispecific antibody comprising a sequence selected from the group consisting of SEQ ID NOs: 39-63; and IL-12. In some embodiments, the cell is modified to express: an anti-BCMA, anti-CD3 bispecific antibody comprising a sequence selected from the group consisting of SEQ ID NOs 75-87; and IL-12. In some embodiments, the cell is modified to express: an anti-BCMA, anti-CD3 bispecific antibody comprising a sequence selected from the group consisting of SEQ ID NOs 88-101; and IL-12. In some embodiments, the cell is modified to express IL-12 and a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 39-63 or 75-101.

In some embodiments, provided is a cell comprising one or more mRNA constructs that collectively encode proteins comprising: an anti-BCMA, anti-CD3 sequence selected from the group consisting of SEQ ID NOs: 39-63; and a protein that is IL-12. In some embodiments, provided is a cell comprising one or more mRNA constructs that collectively encode proteins comprising: a sequence selected from the group consisting of SEQ ID NOs: 75-87; and a protein that is IL-12. In some embodiments, provided is a cell comprising one or more mRNA constructs that collectively encode proteins comprising: a sequence selected from the group consisting of SEQ ID NOs: 88-101; and a protein that is IL-12. In some embodiments, the cell comprises one or more RNA constructs that collectively encode a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 39-63 or 75-101, and a protein that is IL-12.

In some embodiments, provided is a cell comprising one or more mRNA constructs that collectively encode proteins comprising: a sequence selected from the group consisting of SEQ ID NOs: 39-63; SEQ ID NO. 103; and SEQ ID NO. 104. In some embodiments, provided is a cell comprising one or more mRNA constructs that collectively encode proteins comprising: a sequence selected from the group consisting of SEQ ID NOs: 75-87; SEQ ID NO. 103; and SEQ ID NO. 104. In some embodiments, provided is a cell comprising one or more mRNA constructs that collectively encode proteins comprising: a sequence selected from the group consisting of SEQ ID NOs: 88-101; SEQ ID NO. 103; and SEQ ID NO. 104. In some embodiments, the cell comprises one or more RNA constructs that collectively encode a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 39-63, 75-87, or 88-101 and SEQ ID NO: 103 and/or SEQ ID NO:104 or a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NO: 103 and/or SEQ ID NO:104.

In some embodiments, provided is a cell comprising one or more mRNA constructs that collectively encode proteins comprising: a sequence selected from the group consisting of SEQ ID NOs: 39-63; and SEQ ID NO. 105. In some embodiments, provided is a cell comprising one or more mRNA constructs that collectively encode proteins comprising: a sequence selected from the group consisting of SEQ ID NOs: 75-87; and SEQ ID NO. 105. In some embodiments, provided is a cell comprising one or more mRNA constructs that collectively encode proteins comprising: a sequence selected from the group consisting of SEQ ID NOs: 88-101; and SEQ ID NO. 105. In some embodiments, the cell comprises one or more RNA constructs that collectively encode a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 39-63, 75-87, or 88-101 and SEQ ID NO: 105 or a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 105.

### Engineered Cells Expressing an Anti-BCMA, Anti-CD3 Bispecific Antibody and CXCR4

In some embodiments, the cell is modified to express: an anti-BCMA, anti-CD3 bispecific antibody comprising a sequence selected from the group consisting of SEQ ID NOS: 39-63 and 75-101; and CXCR4. In some embodiments, the cell is modified to express: a bispecific antibody comprising a sequence selected from the group consisting of SEQ ID NOS: 39-63; and CXCR4. In some embodiments, the cell is modified to express: a bispecific antibody comprising a sequence selected from the group consisting of SEQ ID NOs 75-87; and CXCR4. In some embodiments, the cell is modified to express: a bispecific antibody comprising a sequence selected from the group consisting of SEQ ID NOs 88-101; and CXCR4. In some embodiments, the cell is modified to express a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 39-63, 75-101 and CXCR4.

In some embodiments, provided is a cell comprising one or more mRNA constructs that collectively encode proteins comprising: a sequence selected from the group consisting of SEQ ID NOs: 39-63; and a protein that is CXCR4. In some embodiments, provided is a cell comprising one or more mRNA constructs that collectively encode proteins comprising: a sequence selected from the group consisting of SEQ ID NOs: 75-87; and a protein that is CXCR4. In some embodiments, provided is a cell comprising one or more mRNA constructs that collectively encode proteins comprising: a sequence selected from the group consisting of SEQ ID NOs: 88-101; and a protein that is CXCR4. In some embodiments, the cell comprises one or more RNA constructs that collectively encode a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 39-63, 75-87, or 88-101 and a protein that is CXCR4.

In some embodiments, provided is a cell comprising one or more mRNA constructs that collectively encode proteins comprising: a sequence selected from the group consisting of SEQ ID NOs: 39-63; and SEQ ID NO. 102. In some embodiments, provided is a cell comprising one or more mRNA constructs that collectively encode proteins comprising: a sequence selected from the group consisting of SEQ ID NOs: 75-87; and SEQ ID NO. 102. In some embodiments, provided is a cell comprising one or more mRNA constructs that collectively encode proteins comprising: a sequence selected from the group consisting of SEQ ID NOs: 88-101; and SEQ ID NO. 102. In some embodiments, the cell comprises one or more RNA constructs that collectively encode a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 39-63, 75-87, or 88-101 and SEQ ID NO: 102 or a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 102.

### Engineered Cells Expressing an Anti-BCMA, Anti-CD3 Bispecific Antibody, IL-12, and CXCR4

In some embodiments, the cell modified to express an anti-BCMA, anti-CD3 bispecific antibody is further modified to express both IL-12 and CXCR4. Thus, in some embodiments, the cell is modified to express: an anti-BCMA, anti-CD3 bispecific antibody comprising a sequence selected from the group consisting of SEQ ID NOs: 39-63 and 75-101; IL-12; and CXCR4. In some embodiments, the cell is modified to express: an anti-BCMA, anti-CD3 bispecific antibody comprising a sequence selected from the group consisting of SEQ ID NOs: 39-63; IL-12; and CXCR4. In some embodiments, the cell is modified to express: an anti-BCMA, anti-CD3 bispecific antibody comprising a sequence selected from the group consisting of SEQ ID NOS 75-87; IL-12; and CXCR4. In some embodiments, the cell is modified to express: an anti-BCMA, anti-CD3 bispecific antibody comprising a sequence selected from the group consisting of SEQ ID NOs: 88-101; IL-12; and CXCR4. In some embodiments, the cell is modified to express a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 39-63, 75-101 and IL12 and CXCR4.

In some embodiments, provided is a cell comprising one or more mRNA constructs that collectively encode proteins comprising: an anti-BCMA, anti-CD3 sequence selected from the group consisting of SEQ ID NOs: 39-63; a protein that is IL-12; and a protein that is CXCR4. In some embodiments, provided is a cell comprising one or more mRNA constructs that collectively encode proteins comprising: a sequence selected from the group consisting of SEQ ID NOs: 75-87; a protein that is IL-12; and a protein that is CXCR4. In some embodiments, provided is a cell comprising one or more mRNA constructs that collectively encode proteins comprising: a sequence selected from the group consisting of SEQ ID NOs: 88-101; a protein that is IL-12; and a protein that is CXCR4. In some embodiments, the cell is modified to express a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 39-63, 75-101 and a protein that is IL-12 and a protein that is CXCR4.

In some embodiments, provided is a cell comprising one or more mRNA constructs that collectively encode proteins comprising: a sequence selected from the group consisting of SEQ ID NOs: 39-63; SEQ ID NO: 102; SEQ ID NO: 103; and SEQ ID NO: 104. In some embodiments, provided is a cell comprising one or more mRNA constructs that collectively encode proteins comprising: a sequence selected from the group consisting of SEQ ID NOs: 75-87; SEQ ID NO: 102; SEQ ID NO: 103; and SEQ ID NO: 104. In some embodiments, provided is a cell comprising one or more mRNA constructs that collectively encode proteins comprising: a sequence selected from the group consisting of SEQ ID NOs: 88-101; SEQ ID NO: 102; SEQ ID NO: 103; and SEQ ID NO: 104. In some embodiments, the cell is modified to express a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 39-63, 75-101 and SEQ ID NO: 102 or a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 102; a SEQ ID NO: 103 or a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 103, and a SEQ ID NO: 104 or a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 104.

In some embodiments, provided is a cell comprising one or more mRNA constructs that collectively encode proteins comprising: a sequence selected from the group consisting of SEQ ID NOs: 39-63; SEQ ID NO: 102; and SEQ ID NO. 105. In some embodiments, provided is a cell comprising one or more mRNA constructs that collectively encode proteins comprising: a sequence selected from the group consisting of SEQ ID NOs: 75-87; SEQ ID NO: 102; and SEQ ID NO. 105. In some embodiments, provided is a cell comprising one or more mRNA constructs that collectively encode proteins comprising: a sequence selected from the group consisting of SEQ ID NOs: 88-101; SEQ ID NO: 102; and SEQ ID NO. 105. In some embodiments, the cell is modified to express a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 39-63, 75-101 and SEQ ID NO: 102 or a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 102, and SEQ ID NO: 105 or a variant or homologue of the bivalent anti-BCMA, anti-CD3 bispecific antibody which are at least about 70% identical, at least about 80% identical, at least about 90% identical, at least about 95% identical, at least about 98% identical, at least about 99% identical, at least about 99.5% identical, or at least about 99.9% to SEQ ID NOs: 105.

In some embodiments the cell secretes sufficient anti-BCMA, anti-CD3 bispecific antibody to induce antimyeloma cytotoxicity, either in vitro in the presence of T cells, or in vivo. In some embodiments, the cell secretes the IL-12 protein(s); in some further embodiments, the co-secretion of IL-12 with the anti-BCMA, anti-CD3 bispecific antibody enhances the antimyeloma cytotoxicity, either in vitro in the presence of T cells, or in vivo. In some embodiments, co-expression of CXCR4 with the anti-BCMA, anti-CD3 bispecific antibody enhances the antimyeloma cytotoxicity, either in vitro in the presence of T cells, or in vivo. In some embodiments of this paragraph, the co-expression of IL-12 and/or CXCR4 synergistically increases antimyeloma cytotoxicity, either in vitro in the presence of T cells, or in vivo.

In some embodiments, the engineered cell can be a stem cell, e.g., an MSC, e.g., an umbilical cord-derived MSC or a bone marrow-derived MSC. In some embodiments, the mRNA is introduced by electroporation. In some embodiments, the mRNA is introduced by use of nanoparticles. In some embodiments, the mRNA is introduced by cell squeezing or mechanical membrane disruption.

In some embodiments, provided is an MSC modified to express an anti-BCMA, anti-CD3 bispecific antibody. In some embodiments, provided is an MSC modified to express an anti-BCMA, anti-CD3 bispecific antibody; and IL-12; and here the IL-12 can optionally be scIL-12. In some embodiments, provided is an MSC modified to express an anti-BCMA, anti-CD3 bispecific antibody; and CXCR4. In some embodiments, provided is an MSC modified to express an anti-BCMA, anti-CD3 bispecific antibody; CXCR4; and IL-12; and here the IL-12 can optionally be scIL-12.

In some embodiments, provided is an MSC comprising an exogenous nucleic acid encoding an anti-BCMA, anti-CD3 bispecific antibody. In some embodiments, provided is an MSC comprising an exogenous nucleic acid encoding an anti-BCMA, anti-CD3 bispecific antibody; and an exogenous nucleic acid encoding IL-12; and here the IL-12 can optionally be scIL-12. In some embodiments, provided is an MSC comprising an exogenous nucleic acid encoding an anti-BCMA, anti-CD3 bispecific antibody; and an exogenous nucleic acid encoding CXCR4. In some embodiments, provided is an MSC comprising an exogenous nucleic acid encoding an anti-BCMA, anti-CD3 bispecific antibody; an exogenous nucleic acid encoding CXCR4; and an exogenous nucleic acid encoding IL-12; and here the IL-12 can optionally be scIL-12. In any of the embodiments of this paragraph, where a genus of nucleic acids is described, the nucleic acid can be a species of that genus identified or recited in this application. e.g., in the sequence listing. In any of the embodiments of this paragraph, a nucleic acid can comprise, or can be, an RNA. In any of the embodiments of this paragraph, a nucleic acid can comprise, or can be, an mRNA. In any of the embodiments of this paragraph, a nucleic acid can comprise, or can be, an DNA.

### Therapeutic Uses of the Engineered Cells

In another aspect, the present invention provides a method for administering the cells, the multispecific antibodies, or composition comprising either the cells or the multispecific antibodies, as described herein. For example, the invention provides a method for stimulating a T cell-mediated immune response to a target cell population (e.g., myeloma cells or pathogenic plasma cells) in a mammal comprising the step of administering to the mammal an inventive cell described herein, e.g., an MSC engineered to express: (1) an anti-BCMA, anti-CD3 bispecific antibody; (2) IL-12; and (3) CXCR4. Alternatively, the present invention provides a method comprising the step of administering to the mammal the supernatant collected from, or proteins isolated from, an inventive cell described herein, e.g., a cell engineered to express: (1) an anti-BCMA, anti-CD3 bispecific antibody; (2) IL-12; and (3) CXCR4.

The engineered cells of the present invention, or a composition comprising such cells, or proteins isolated from such cells, may be used, or may be administered to a subject in need thereof, to provide anti-tumor immunity; to treat or prevent cancer; to treat or prevent autoimmune condition; or to treat or prevent an allergic condition. In some embodiments, the cancer is multiple myeloma, Hodgkin lymphoma, non-Hodgkin lymphoma, a leukemia, or glioblastoma. In some embodiments, the autoimmune condition is myasthenia gravis, neuromyelitis optica, chronic inflammatory demyelinating polyradiculoneuropathy (CIDP), systemic lupus erythematosus, rheumatoid arthritis, pemphigus, psoriasis, inflammatory bowel disease, celiac sprue, pernicious anemia, idiopathic thrombocytopenia purpura, scleroderma, Graves' disease, Sjögren syndrome, Goodpasture syndrome, or type 1 diabetes. In some embodiments, the allergic condition is anaphylaxis, asthma, food allergy, stinging insect allergy, drug allergy, allergic rhinitis, urticaria, angioedema, eczema, atopic dermatitis, contact dermatitis, and cosinophilic esophagitis.

The engineered cells of the present invention may be administered either alone, or as a composition (e.g., a pharmaceutical composition) in combination with diluents and/or with other components such as IL-2 or other cytokines or cell populations. Briefly, pharmaceutical compositions of the present invention may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions of the present invention can be formulated for intravenous administration.

Pharmaceutical compositions of the present invention may be administered as appropriate for the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the inventive engineered cells described herein may be administered at a dosage of 104 to 10⁹ cells/kg body weight, preferably 105 to 10⁷ cells/kg body weight, including all integer values within those ranges. T cell compositions may also be administered multiple times at these dosages. The cells can be administered by using infusion techniques that are commonly known in immunotherapy (scc, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

The administration of the subject compositions may be carried out in any convenient manner, including by aerosol inhalation, injection, ingestion, transfusion, implantation, or transplantation. The compositions described herein may be administered to a patient subcutaneously, intradermally, intratumorally, intranodally, intramedullary, intramuscularly, by intravenous (i.v.) injection, or intraperitoneally. In some embodiments, the immune cell (e.g., T cell) compositions of the present invention are administered to a patient by intradermal or subcutaneous injection. In another embodiment, the engineered cell compositions of the present invention are preferably administered by i.v. injection. The compositions of engineered cells may be injected directly into a tumor, lymph node, or site of disease.

In certain embodiments of the present invention, the engineered cells are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS (multiple sclerosis) patients or efalizumab treatment for psoriasis patients or other treatments for PML (progressive multifocal leukoencephalopathy) patients. In further embodiments, the cells of the invention may be used in combination with chemotherapy, radiation, immunosuppressive agents, such as cyclosporin, azathioprine, methotrexate, mycophenolate, and FK506, antibodies, or other immunoablative agents such as CAMPATH (alemtuzumab), anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). In a further embodiment, the cell compositions of the present invention are administered to a patient in conjunction with (e.g., before, simultaneously or following) bone marrow transplantation, white blood cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

Wherever in this disclosure there is a described a method of administering cells or a composition to a subject to treat or prevent a disease, likewise contemplated is the use of such cells or composition for the prevention or treatment of the disease.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXEMPLARY EMBODIMENTS

Some embodiments of the invention are:

Embodiment 1: A protein comprising the sequence of SEQ ID NO: 2, or an amino acid sequence that is at least about 80% identical to SEQ ID NO: 2.

Embodiment 2: A protein comprising the sequence of SEQ ID NO: 3, or an amino acid sequence that is at least about 80% identical to SEQ ID NO: 3.

Embodiment 3: A protein comprising the sequence of SEQ ID NO: 4, or an amino acid sequence that is at least about 80% identical to SEQ ID NO: 4.

Embodiment 4: A protein comprising the sequence of SEQ ID NO: 5, or an amino acid sequence that is at least about 80% identical to SEQ ID NO: 5.

Embodiment 5: A protein comprising the sequence of SEQ ID NO: 6, or an amino acid sequence that is at least about 80% identical to SEQ ID NO: 6.

Embodiment 6: A protein comprising the sequence of SEQ ID NO: 7, or an amino acid sequence that is at least about 80% identical to SEQ ID NO: 7.

Embodiment 7: A protein comprising the sequence of SEQ ID NO: 8, or an amino acid sequence that is at least about 80% identical to SEQ ID NO: 8.

Embodiment 8: A protein comprising the sequence of SEQ ID NO: 9, or an amino acid sequence that is at least about 80% identical to SEQ ID NO: 9.

Embodiment 9: A protein comprising the sequence of SEQ ID NO: 10, or an amino acid sequence that is at least about 80% identical to SEQ ID NO: 10.

Embodiment 10: A protein comprising the sequence of SEQ ID NO: 11, or an amino acid sequence that is at least about 80% identical to SEQ ID NO: 11.

Embodiment 11: A protein comprising the sequence of SEQ ID NO: 12, or an amino acid sequence that is at least about 80% identical to SEQ ID NO: 12.

Embodiment 12: A protein comprising the sequence of SEQ ID NO: 13, or an amino acid sequence that is at least about 80% identical to SEQ ID NO: 13.

Embodiment 13: A protein comprising the sequence of SEQ ID NO: 14, or an amino acid sequence that is at least about 80% identical to SEQ ID NO: 14.

Embodiment 14: A protein comprising the sequence of SEQ ID NO: 15, or an amino acid sequence that is at least about 80% identical to SEQ ID NO: 15.

Embodiment 15: A protein comprising the sequence of SEQ ID NO: 16, or an amino acid sequence that is at least about 80% identical to SEQ ID NO: 16.

Embodiment 16: A protein comprising the sequence of SEQ ID NO: 17, or an amino acid sequence that is at least about 80% identical to SEQ ID NO: 17.

Embodiment 17: A protein comprising the sequence of SEQ ID NO: 18, or an amino acid sequence that is at least about 80% identical to SEQ ID NO: 18.

Embodiment 18: A protein comprising the sequence of SEQ ID NO: 19, or an amino acid sequence that is at least about 80% identical to SEQ ID NO: 19.

Embodiment 19: A protein comprising the sequence of SEQ ID NO: 20, or an amino acid sequence that is at least about 80% identical to SEQ ID NO: 20.

Embodiment 20: A protein comprising the sequence of SEQ ID NO: 21, or an amino acid sequence that is at least about 80% identical to SEQ ID NO: 21.

Embodiment 21: A protein comprising the sequence of SEQ ID NO: 22, or an amino acid sequence that is at least about 80% identical to SEQ ID NO: 22.

Embodiment 22: A protein comprising the sequence of SEQ ID NO: 23, or an amino acid sequence that is at least about 80% identical to SEQ ID NO: 23.

Embodiment 23: A protein comprising the sequence of SEQ ID NO: 24, or an amino acid sequence that is at least about 80% identical to SEQ ID NO: 24.

Embodiment 24: A protein comprising the sequence of SEQ ID NO: 25, or an amino acid sequence that is at least about 80% identical to SEQ ID NO: 25.

Embodiment 25: A protein comprising the sequence of SEQ ID NO: 26, or an amino acid sequence that is at least about 80% identical to SEQ ID NO: 26.

Embodiment 26: A protein comprising the sequence of SEQ ID NO: 27, or an amino acid sequence that is at least about 80% identical to SEQ ID NO: 27.

Embodiment 27: A protein comprising the sequence of SEQ ID NO: 28, or an amino acid sequence that is at least about 80% identical to SEQ ID NO: 28.

Embodiment 28: A protein comprising the sequence of SEQ ID NO: 29, or an amino acid sequence that is at least about 80% identical to SEQ ID NO: 29.

Embodiment 29: A protein comprising the sequence of SEQ ID NO: 30, or an amino acid sequence that is at least about 80% identical to SEQ ID NO: 30.

Embodiment 30: A protein comprising the sequence of SEQ ID NO: 31, or an amino acid sequence that is at least about 80% identical to SEQ ID NO: 31.

Embodiment 31: A protein comprising the sequence of SEQ ID NO: 32, or an amino acid sequence that is at least about 80% identical to SEQ ID NO: 32.

Embodiment 32: A protein comprising the sequence of SEQ ID NO: 33, or an amino acid sequence that is at least about 80% identical to SEQ ID NO: 33.

Embodiment 33: A protein comprising the sequence of SEQ ID NO: 34, or an amino acid sequence that is at least about 80% identical to SEQ ID NO: 34.

Embodiment 34: A protein comprising the sequence of SEQ ID NO: 35, or an amino acid sequence that is at least about 80% identical to SEQ ID NO: 35.

Embodiment 35: A protein comprising the sequence of SEQ ID NO: 36, or an amino acid sequence that is at least about 80% identical to SEQ ID NO: 36.

Embodiment 36: A protein comprising the sequence of SEQ ID NO: 37, or an amino acid sequence that is at least about 80% identical to SEQ ID NO: 37.

Embodiment 37: A protein comprising the sequence of SEQ ID NO: 38, or an amino acid sequence that is at least about 80% identical to SEQ ID NO: 38.

Embodiment 38: A protein comprising a sequence selected from the group consisting of SEQ ID NOs: 2-37.

Embodiment 39: A protein comprising the sequence of SEQ ID NO: 41, or an amino acid sequence that is at least about 80% identical to SEQ ID NO: 41.

Embodiment 40: The protein of any one of Embodiments 1-38, the protein further comprising the sequence of SEQ ID NO: 39 or an amino acid sequence that is at least about 80% identical to SEQ ID NO: 39.

Embodiment 41: The protein of any one of Embodiments 1-38, the protein further comprising the sequence of SEQ ID NO: 40 or an amino acid sequence that is at least about 80% identical to SEQ ID NO: 40.

Embodiment 42: The protein of any one of Embodiments 1-38, the protein further comprising the sequence of SEQ ID NO: 41 or an amino acid sequence that is at least about 80% identical to SEQ ID NO: 41.

Embodiment 43: The protein of any one of Embodiments 1-38, the protein further comprising the sequence of SEQ ID NO: 42 or an amino acid sequence that is at least about 80% identical to SEQ ID NO: 42.

Embodiment 44: The protein of any one of Embodiments 1-38, the protein further comprising the sequence of SEQ ID NO: 43 or an amino acid sequence that is at least about 80% identical to SEQ ID NO: 43.

Embodiment 45: The protein of any one of Embodiments 1-38, the protein further comprising the sequence of SEQ ID NO: 44 or an amino acid sequence that is at least about 80% identical to SEQ ID NO: 44.

Embodiment 46: The protein of any one of Embodiments 1-38, the protein further comprising the sequence of SEQ ID NO: 45 or an amino acid sequence that is at least about 80% identical to SEQ ID NO: 45.

Embodiment 47: The protein of any one of Embodiments 1-38, the protein further comprising the sequence of SEQ ID NO: 46 or an amino acid sequence that is at least about 80% identical to SEQ ID NO: 46.

Embodiment 48: The protein of any one of Embodiments 1-38, the protein further comprising the sequence of SEQ ID NO: 47 or an amino acid sequence that is at least about 80% identical to SEQ ID NO: 47.

Embodiment 49: The protein of any one of Embodiments 1-38, the protein further comprising the sequence of SEQ ID NO: 48 or an amino acid sequence that is at least about 80% identical to SEQ ID NO: 48.

Embodiment 50: The protein of any one of Embodiments 1-38, the protein further comprising the sequence of SEQ ID NO: 49 or an amino acid sequence that is at least about 80% identical to SEQ ID NO: 49.

Embodiment 51: The protein of any one of Embodiments 1-38, the protein further comprising the sequence of SEQ ID NO: 50 or an amino acid sequence that is at least about 80% identical to SEQ ID NO: 50.

Embodiment 52: The protein of any one of Embodiments 1-38, the protein further comprising the sequence of SEQ ID NO: 51 or an amino acid sequence that is at least about 80% identical to SEQ ID NO: 51.

Embodiment 53: The protein of any one of Embodiments 1-38, the protein further comprising the sequence of SEQ ID NO: 52 or an amino acid sequence that is at least about 80% identical to SEQ ID NO: 52.

Embodiment 54: The protein of any one of Embodiments 1-38, the protein further comprising the sequence of SEQ ID NO: 53 or an amino acid sequence that is at least about 80% identical to SEQ ID NO: 53.

Embodiment 55: The protein of any one of Embodiments 1-38, the protein further comprising the sequence of SEQ ID NO: 54 or an amino acid sequence that is at least about 80% identical to SEQ ID NO: 54.

Embodiment 56: The protein of any one of Embodiments 1-38, the protein further comprising the sequence of SEQ ID NO: 55 or an amino acid sequence that is at least about 80% identical to SEQ ID NO: 55.

Embodiment 57: The protein of any one of Embodiments 1-38, the protein further comprising the sequence of SEQ ID NO: 56 or an amino acid sequence that is at least about 80% identical to SEQ ID NO: 56.

Embodiment 58: The protein of any one of Embodiments 1-38, the protein further comprising the sequence of SEQ ID NO: 57 or an amino acid sequence that is at least about 80% identical to SEQ ID NO: 57.

Embodiment 59: The protein of any one of Embodiments 1-38, the protein further comprising the sequence of SEQ ID NO: 58 or an amino acid sequence that is at least about 80% identical to SEQ ID NO: 58.

Embodiment 60: The protein of any one of Embodiments 1-38, the protein further comprising the sequence of SEQ ID NO: 59 or an amino acid sequence that is at least about 80% identical to SEQ ID NO: 59.

Embodiment 61: The protein of any one of Embodiments 1-38, the protein further comprising the sequence of SEQ ID NO: 60 or an amino acid sequence that is at least about 80% identical to SEQ ID NO: 60.

Embodiment 62: The protein of any one of Embodiments 1-38, the protein further comprising the sequence of SEQ ID NO: 61 or an amino acid sequence that is at least about 80% identical to SEQ ID NO: 61.

Embodiment 63: The protein of any one of Embodiments 1-38, the protein further comprising the sequence of SEQ ID NO: 62 or an amino acid sequence that is at least about 80% identical to SEQ ID NO: 62.

Embodiment 64: The protein of any one of Embodiments 1-38, the protein further comprising the sequence of SEQ ID NO: 63 or an amino acid sequence that is at least about 80% identical to SEQ ID NO: 63.

Embodiment 65: The protein of any one of Embodiments 1-38, the protein further comprising the sequence of SEQ ID NO: 64 or an amino acid sequence that is at least about 80% identical to SEQ ID NO: 64.

Embodiment 66: The protein of any one of Embodiments 1-38, the protein further comprising a sequence selected from the group consisting of SEQ ID NOs: 39-64.

Embodiment 67: A protein that is a variant of any one of Embodiments 1-66, wherein one or more linkers is each substituted by a linker independently selected from SEQ ID NOs: 65-68 and 70-73, Sequence A.

Embodiment 68: A protein comprising the sequence of SEQ ID NO: 75, or an amino acid sequence that is at least about 80% identical to SEQ ID NO: 75.

Embodiment 69: A protein comprising the sequence of SEQ ID NO: 76, or an amino acid sequence that is at least about 80% identical to SEQ ID NO: 76.

Embodiment 70: A protein comprising the sequence of SEQ ID NO: 77, or an amino acid sequence that is at least about 80% identical to SEQ ID NO: 77.

Embodiment 71: A protein comprising the sequence of SEQ ID NO: 78, or an amino acid sequence that is at least about 80% identical to SEQ ID NO: 78.

Embodiment 72: A protein comprising the sequence of SEQ ID NO: 79, or an amino acid sequence that is at least about 80% identical to SEQ ID NO: 79.

Embodiment 73: A protein comprising the sequence of SEQ ID NO: 80, or an amino acid sequence that is at least about 80% identical to SEQ ID NO: 80.

Embodiment 74: A protein comprising the sequence of SEQ ID NO: 81, or an amino acid sequence that is at least about 80% identical to SEQ ID NO: 81.

Embodiment 75: A protein comprising the sequence of SEQ ID NO: 82, or an amino acid sequence that is at least about 80% identical to SEQ ID NO: 82.

Embodiment 76: A protein comprising the sequence of SEQ ID NO: 83, or an amino acid sequence that is at least about 80% identical to SEQ ID NO: 83.

Embodiment 77: A protein comprising the sequence of SEQ ID NO: 84, or an amino acid sequence that is at least about 80% identical to SEQ ID NO: 84.

Embodiment 78: A protein comprising the sequence of SEQ ID NO: 85, or an amino acid sequence that is at least about 80% identical to SEQ ID NO: 85.

Embodiment 79: A protein comprising the sequence of SEQ ID NO: 86, or an amino acid sequence that is at least about 80% identical to SEQ ID NO: 86.

Embodiment 80: A protein comprising the sequence of SEQ ID NO: 87, or an amino acid sequence that is at least about 80% identical to SEQ ID NO: 87.

Embodiment 81: A protein comprising the sequence of SEQ ID NO: 88, or an amino acid sequence that is at least about 80% identical to SEQ ID NO: 80.

Embodiment 82: A protein comprising the sequence of SEQ ID NO: 89, or an amino acid sequence that is at least about 80% identical to SEQ ID NO: 89.

Embodiment 83: A protein comprising the sequence of SEQ ID NO: 90, or an amino acid sequence that is at least about 80% identical to SEQ ID NO: 90.

Embodiment 84: A protein comprising the sequence of SEQ ID NO: 91, or an amino acid sequence that is at least about 80% identical to SEQ ID NO: 91.

Embodiment 85: A protein comprising the sequence of SEQ ID NO: 92, or an amino acid sequence that is at least about 80% identical to SEQ ID NO: 92.

Embodiment 86: A protein comprising the sequence of SEQ ID NO: 93, or an amino acid sequence that is at least about 80% identical to SEQ ID NO: 93.

Embodiment 87: A protein comprising the sequence of SEQ ID NO: 94, or an amino acid sequence that is at least about 80% identical to SEQ ID NO: 94.

Embodiment 88: A protein comprising the sequence of SEQ ID NO: 95, or an amino acid sequence that is at least about 80% identical to SEQ ID NO: 95.

Embodiment 89: A protein comprising the sequence of SEQ ID NO: 96, or an amino acid sequence that is at least about 80% identical to SEQ ID NO: 96.

Embodiment 90: A protein comprising the sequence of SEQ ID NO: 97, or an amino acid sequence that is at least about 80% identical to SEQ ID NO: 97.

Embodiment 91: A protein comprising the sequence of SEQ ID NO: 98, or an amino acid sequence that is at least about 80% identical to SEQ ID NO: 98.

Embodiment 92: A protein comprising the sequence of SEQ ID NO: 99, or an amino acid sequence that is at least about 80% identical to SEQ ID NO: 99.

Embodiment 93: A protein comprising the sequence of SEQ ID NO: 100, or an amino acid sequence that is at least about 80% identical to SEQ ID NO: 100.

Embodiment 94: A protein comprising the sequence of SEQ ID NO: 101, or an amino acid sequence that is at least about 80% identical to SEQ ID NO: 101.

Embodiment 95: A protein comprising a sequence selected from the group consisting of SEQ ID NOs: 75-101.

Embodiment 96: A protein comprising a sequence selected from the group consisting of SEQ ID NOs: 75-87.

Embodiment 97: A protein comprising a sequence selected from the group consisting of SEQ ID NOs: 88-101.

Embodiment 98: The protein of any one of Embodiments 1-97, wherein the protein is an anti-BCMA, anti-CD3 bispecific antibody.

Embodiment 99: The protein of Embodiment 98, wherein the protein is a bivalent antibody.

Embodiment 100: The protein of Embodiment 98, wherein the protein is a trivalent antibody.

Embodiment 101: The protein of Embodiment 98, wherein the protein is a tetravalent antibody.

Embodiment 102: The protein of any one of Embodiments 1-97, wherein the protein is a trispecific binding that specifically binds to BCMA, CD3, and a third antigen.

Embodiment 103: The protein of Embodiment 102, wherein the third antigen is albumin.

Embodiment 104: A polynucleotide encoding the protein of any one of Embodiments 1-103.

Embodiment 105: A polynucleotide encoding two or more proteins selected from the group consisting of the proteins of Embodiments 1-97.

Embodiment 106: A mammalian cell comprising a first exogenous mRNA encoding a protein of any one of claims 1-103.

Embodiment 107: The mammalian cell of claim 106, wherein the cell is a mesenchymal stem cell.

Embodiment 108: An mRNA vector encoding the protein of any one of Embodiments 1-103.

Embodiment 109: An mRNA vector encoding one or more proteins selected from the group consisting of the proteins of Embodiments 1-103.

Embodiment 110: A DNA vector encoding the protein of any one of Embodiments 1-103.

Embodiment 111: A DNA vector encoding one or more proteins selected from the group consisting of the proteins of Embodiments 1-103.

Embodiment 112: A mammalian cell modified to express: (1) an anti-BCMA, anti-CD3 bispecific antibody; (2) IL-12 of a synthetic analog thereof; and (3) CXCR4 or a synthetic analog thereof.

Embodiment 113: A mammalian cell modified to express the protein of any one of Embodiments 1-103.

Embodiment 114: The cell of Embodiment 113, wherein the cell is further modified to express IL-12 or an analog thereof.

Embodiment 115: The cell of Embodiment 113, wherein the cell secretes the IL-12 or the analog thereof.

Embodiment 116: the cell of Embodiment 113, wherein the cell is further modified to express CXC4.

Embodiment 117: the cell of Embodiments 113, wherein the cell is further modified to express: IL-12 or an analog thereof; and CXC4.

Embodiment 118: the cell of Embodiment 117, wherein the cell secretes the IL-12 or the analog thereof.

Embodiment 119: A mammalian cell comprising a polynucleotide of Embodiment 104.

Embodiment 120: The mammalian cell of Embodiment 140, wherein the polynucleotide is an artificially synthesized mRNA.

Embodiment 121: A mammalian cell comprising a polynucleotide of Embodiment 105.

Embodiment 122: The mammalian cell of Embodiment 121, wherein the polynucleotide is an artificially synthesized mRNA.

Embodiment 123: The mammalian cell of any one of Embodiments 110-120, wherein the cell is modified by electroporation with one or more heterologous polynucleotides.

Embodiment 124: The mammalian cell of any one of Embodiments 112-123, wherein the cell is a stem cell.

Embodiment 125: The mammalian cell of any one of Embodiments 112-123, wherein the cell is a mesenchymal stem cell.

Embodiment 126: The mammalian cell of any one of Embodiments 112-123, wherein the cell is an umbilical cord mesenchymal stem cell.

Embodiment 127: The mammalian cell of any one of Embodiments 112-123, wherein the cell is a CD34+ stem cell.

Embodiment 128: The mammalian cell of any one of Embodiments 112-123, wherein the cell is a T cell.

Embodiment 129: The mammalian cell of any one of Embodiments 112-123, wherein the cell is a natural killer (NK) cell.

Embodiment 130: A mesenchymal stem cell modified to express an anti-BCMA, anti-CD3 bispecific antibody.

Embodiment 131: A mesenchymal stem cell modified to express: an anti-BCMA, anti-CD3 bispecific antibody; and IL-12.

Embodiment 132: A mesenchymal stem cell modified to express: an anti-BCMA, anti-CD3 bispecific antibody; IL-12; and CXCR4.

Embodiment 133: The cell of Embodiment 131 or 132, wherein the IL-12 is scIL-12.

Embodiment 134: A mesenchymal stem cell modified to express an anti-BCMA, anti-CD3 bispecific antibody.

Embodiment 135: A mesenchymal stem cell comprising: an exogenous nucleic acid encoding an anti-BCMA, anti-CD3 bispecific antibody; and an exogenous nucleic acid encoding IL-12.

Embodiment 136: A mesenchymal stem cell comprising: an exogenous nucleic acid encoding an anti-BCMA, anti-CD3 bispecific antibody; an exogenous nucleic acid encoding IL-12; and an exogenous nucleic acid encoding CXCR4.

Embodiment 137: The mesenchymal stem cell of either Embodiment 135 or 136, wherein the IL-12 is scIL-12.

Embodiment 138: The mesenchymal stem cell of any one of Embodiments 135-137, wherein the exogenous nucleic acids comprise mRNA.

Embodiment 139: The mesenchymal stem cell of any one of Embodiments 135-137, wherein the exogenous nucleic acids are mRNA.

Embodiment 140: The mesenchymal stem cell of any one of Embodiments 135-137, wherein the exogenous nucleic acids comprise DNA.

Embodiment 141: The mesenchymal stem cell of any one of Embodiments 135-137, wherein the exogenous nucleic acids are DNA.

Embodiment 142: The mesenchymal stem cell of any one of Embodiments 130-141, wherein the anti-BCMA, anti-CD3 bispecific antibody is encoded by a protein of any one of Embodiments 1-103.

Embodiment 143: A cell therapy product comprising a plurality of cells selected from any one of Embodiments 112-142; and a pharmaceutically acceptable carrier

Embodiment 144: A method of treating disease in a subject in need thereof, the method comprising administering to the patient a cell of any one of Embodiments 112-142.

Embodiment 145: A method of preventing disease in a subject in need thereof, the method comprising administering to the patient a cell of any one of Embodiments 112-142.

Embodiment 146: A method of treating disease in a subject in need thereof, the method comprising administering to the patient a therapeutically effective amount of the cell therapy of Embodiment 141.

Embodiment 147: A method of preventing disease in a subject in need thereof, the method comprising administering to the patient a therapeutically effective amount of the cell therapy of Embodiment 143.

Embodiment 148: The method of any one of Embodiments 144-147, wherein the disease is myeloma.

Embodiment 149: The method of any one of Embodiments 144-147, wherein the disease is an autoimmune disease.

Embodiment 150: The method of Embodiment 149, wherein the disease is selected from the group consisting of: myasthenia gravis, neuromyelitis optica, chronic inflammatory demyelinating polyradiculoneuropathy (CIDP), systemic lupus erythematosus (SLE), rheumatoid arthritis, blistering skin diseases, pemphigus, psoriasis, inflammatory bowel disease, celiac sprue, pernicious anemia, idiopathic thrombocytopenia purpura, scleroderma, Graves' disease, Sjögren syndrome, Goodpasture syndrome, and type 1 diabetes.

Embodiment 151: The method of any one of Embodiments 144-147, wherein the disease is an allergic disorder.

Embodiment 152: The method of Embodiment 151, wherein the disease is selected from the group consisting of: anaphylaxis, asthma, food allergy, stinging insect allergy, drug allergy, allergic rhinitis, urticaria, angioedema, eczema, atopic dermatitis, contact dermatitis, and eosinophilic esophagitis.

Embodiment 153: Use of a cell of any one of Embodiments 112-142 for the treatment of a disease.

Embodiment 154: Use of a cell of any one of Embodiments 112-142 for the prevention of a disease.

Embodiment 155: Use of a product of Embodiment 143 for the treatment of a disease.

Embodiment 156: Use of a product of Embodiment 143 for the prevention of a disease.

Embodiment 157: The use of any one of Embodiments 153-156, wherein the disease is myeloma.

Embodiment 158: The use of any one of Embodiments 153-156, wherein the disease is an autoimmune disease.

Embodiment 159: The use of Embodiment 158, wherein the disease is selected from the group consisting of: myasthenia gravis, neuromyelitis optica, chronic inflammatory demyelinating polyradiculoneuropathy (CIDP), systemic lupus erythematosus (SLE), rheumatoid arthritis, blistering skin diseases, pemphigus, psoriasis, inflammatory bowel disease, celiac sprue, pernicious anemia, idiopathic thrombocytopenia purpura, scleroderma, Graves' disease, Sjögren syndrome, Goodpasture syndrome, and type 1 diabetes.

Embodiment 160: The use of any one of Embodiments 153-156, wherein the disease is an allergic disorder.

Embodiment 161: The use of Embodiment 160, wherein the disease is selected from the group consisting of: anaphylaxis, asthma, food allergy, stinging insect allergy, drug allergy, allergic rhinitis, urticaria, angioedema, eczema, atopic dermatitis, contact dermatitis, and eosinophilic esophagitis.

### EXAMPLES

Without further elaboration, it is believed that one of ordinary skill in the art can, based on the above description, utilize the present disclosure to its fullest extent. Nevertheless, in order that the invention described herein may be more fully understood, the following examples are set forth. The synthetic examples described in this application are offered to illustrate the compounds and methods provided herein and are not to be construed in any way as limiting their scope. All publications cited herein are incorporated by reference for the purposes or subject referenced herein.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein are meant to be non-limiting. In describing and claiming the present invention, the following terminology will be used.

### Example 1: Preparation of Therapeutic MSCs that Produce an Anti-BCMA, Anti-CD3 Bispecific Antibody, IL-12, and CXCR4

The following example is of inventive MSCs, herein called “Triceratops”, produced by introduction of an inventive combination of mRNA constructs. The mRNA constructs encode, and the MSCs express: (1) a novel anti-BCMA, anti-CD3 bispecific antibody; (2) a modified form of IL-12; and (3) CXCR4.

### Production of Triceratops MSCs

Three separate mRNA constructs, corresponding to SEQ ID: 106 (bispecific antibody), SEQ ID: 107 (fusion of both IL-12 subunits), and SEQ ID: 108 (CXCR4), were generated by in vitro transcription from DNA plasmids. The in vitro transcription was performed by T7 RNA polymerase from a PCR amplified DNA template. A polyadenine tail of 181 adenine nucleotides was added to each mRNA through inclusion of a complementary sequence in the reverse primer of the PCR reaction. A 7-methylguanosine cap was incorporated at the 5′ end of each mRNA during the co-transcriptional RNA synthesis.

The first mRNA construct which encoded an inventive, anti-BCMA, anti-CD3 bispecific antibody, comprised, from 5′ to 3′: a 5′ cap; an mRNA sequence of SEQ ID NO: 106; and a 3′ polyadenine tail of 181 adenine units.

The second mRNA construct, which encoded an engineered protein (comprising alpha and beta subunits of human IL-12 connected by a linker), comprised, from 5′ to 3′: a 5′ cap; an mRNA sequence of SEQ ID NO: 107; and a 3′ polyadenine tail of 181 adenine units.

The third mRNA construct, which encoded human CXCR4 protein, comprised, from 5′ to 3′: a 5′ cap; an mRNA sequence of SEQ ID NO: 108; and a 3′ polyadenine tail of 181 adenine units.

To introduce the RNA constructs into the MSCs, about 1 million MSCs were isolated from human umbilical cord and expanded in 2D vessels by culture at 37° C. with 5% CO₂ in ROOSTERBIO® media for 4 passages. The cells were resuspended in 100 μL transfection buffer (known in the art) and simultaneously transfected with a mixture of the three aforementioned mRNA constructs (7.5 μg, 1.0 μg, 1.5 μg, respectively) by electroporation (4D NUCLEOFECTOR®, Lonza) according to manufacturer's instructions. The modified MSCs were returned to culture for 16 hours.

### Characterization of the Triceratops MSCs

Triceratops MSCs obtained from the above-described process were tested for viability, bispecific antibody production, IL-12 secretion, SDF1 chemotaxis, in vitro antimyeloma cytotoxicity, in vitro IFNγ induction in PBMC, and in vivo antimyeloma activity.

To test viability, a sample of Triceratops MSCs was mixed with acridine orange and propidium iodide and analyzed using a NEXCELOM® K2 cellometer. The following result was observed:

| | |
|---|---|
| Viability | 93.7% |

To test bispecific antibody production, supernatant was collected from cultures of Triceratops MSCs (or unmodified control MSCs) 24 hours after electroporation. The supernatant was assessed for bispecific antibody concentration using an enzyme-linked immunosorbent assay. The following result was observed:

| | Bispecific antibody |
|---|---|
| Condition | concentration (ng/ml) |
| Unmodified control | 0 |
| MSCs | |
| Triceratops MSCs | 43.14 |

To test IL-12 production, supernatant was collected from cultures of Triceratops MSC (or unmodified control MSC) 24 hours after electroporation. The supernatant was assessed for IL-12 concentration using an anti-human Interleukin-12 specific ELISA (Biolegend). The following result was observed:

| | IL-12 concentration |
|---|---|
| Condition | (ng/ml) |
| Unmodified control | 0 |
| MSCs | |
| Triceratops MSCs | 5.05 |

To test CXCR4-mediated chemotaxis along a ligand (SDF-1) gradient, the Triceratops MSCs (or unmodified control cells) were plated in 96-well plates and cultured for 8 hours to induce spheroid formation. Spheroids were placed in transwell cultures for 2 days with the lower chamber of the transwell supplemented with 0, 10, or 100 ng/ml recombinant SDF1. Transwells were then fixed, and cells were stained with crystal violet. After mechanical removal of cells on the upper side of the transwell, migrated cells were counted using bright field microscopy.

| | Cell migration |
|---|---|
| Condition | (counts per field) |
| Unmodified control | 8.25 ± 3.5% |
| MSCs | |
| Triceratops MSCs | |
| No SDF1 | 40.0 ± 4.4% |
| 10 ng/mL SDF1 | 62.0 ± 7.8% |
| 100 ng/mL SDF1 | 60.8 ± 10.8% |

To test the Triceratops MSCs' capacity to induce in vitro antimyeloma cytotoxicity and IFNγ secretion by PBMC, 100 μL per well of supernatant from Triceratops MSCs or unmodified control MSCs (tested at a final 1:1000 dilution) was co-incubated with 25,000 cells from a BCMA+ myeloma cell line expressing green fluorescent protein (MM.1S-GFP) plus 100,000 PBMCs. The co-culture was incubated for 36 hours. Supernatant (50 μl per well) was then collected and analyzed for MM.1S-GFP cytotoxicity (by flow cytometry) and IFNγ concentration. The following results were observed:

| Condition | Cytotoxicity | IFNγ (ng/ml) |
|---|---|---|
| Unmodified control | ~0 | 0 |
| MSCs | | |
| Triceratops MSCs | 87% | 17 |

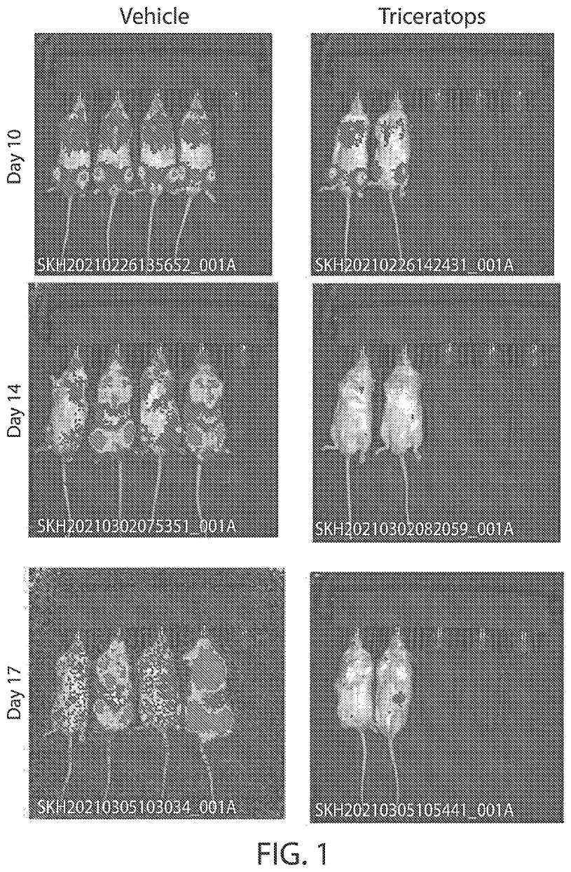

To test the Triceratops MSCs' in vivo antimyeloma activity, NSG (NOD SCID) mice were administered 2 million MM1S-fluc myeloma cells on day 0 and 20 million activated human, pan-CD3 T cells on day 5. Triceratops MSCs (0.15, 0.45 or 1.5 million cells), unmodified control MSCs (1.5 million cells) or inert vehicle (control) were administered intravenously 7, 10 and 14 days after administration of myeloma cells. Myeloma tumor burden was assessed on Days 14 and 17 by bioluminescent flux (FIG. 1). The following results were observed:

| | Tumor Burden (flux, p/s) | | | |
|---|---|---|---|---|
| Condition | Day 14 | | Day 17 | |
| Unmodified control | 11.4 | million | 4.9 | million |
| MSCs | | | | |
| Vehicle control | 53.4 | million | 175.5 | million |
| Triceratops MSCs | | | | |
| 0.15 million | 0.8 | million | 3.0 | million |
| 0.45 million | 0.3 | million | 1.9 | million |
| 1.5 million | 4.2 | million | 3.6 | million |

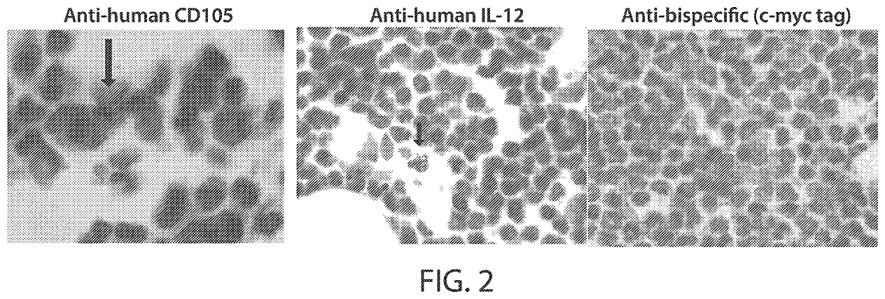

Also, mice were treated with a version of Triceratops cells wherein the anti-BCMA, anti-CD3 bispecific antibody included a myc/flag tag (SEQ ID NO: 141). A representative biopsy, shown in FIG. 2, demonstrates that infused Triceratops cells (which are CD105+) localized to the bone marrow, secreted anti-BCMA, anti-CD3 bispecific antibody, and secreted human IL-12 engineered protein.

In conclusion, Triceratops MSCs were prepared by electroporation of an inventive combination of three mRNA constructs that encoded: (1) a novel anti-BCMA, anti-CD3 bispecific antibody; (2) a novel, modified form of soluble IL-12; and (3) CXCR4. The Triceratops MSCs were viable; secreted the bispecific antibody and IL-12, exhibited SDF1 chemotaxis; and potently and extensively killed myeloma cells both in vitro and in vivo.

### Example 2: Discovery and Functional Comparison of Novel Bispecific Antibodies

A series of in vitro transcribed mRNA encoding the following bispecific antibodies was produced, with characteristics as follows:

| | Protein Description (detailed description does not necessarily include all |
|---|---|
| Name | amino acids, e.g., linkers) |
| Diplodocus-1 | SEQ ID NO: 75 |
| (anti-CD19 | Signal sequence (SEQ ID NO: 109), Flag tag, anti-CD19 scFv (see U.S. |
| control) | Pat. No. 7,575,923), linker (SEQ ID NO: 66), anti-CD3 heavy chain (SEQ |
| | ID NO: 110), linker (SEQ ID NO: 67), anti-CD3 light chain (SEQ ID NO: |
| | 111), His tag. |
| Diplodocus-2 | SEQ ID NO: 76 |
| | Signal sequence (SEQ ID NO: 109), anti-BCMA scFv (SEQ ID NO: 41), |
| | linker (SEQ ID NO: 66), anti-CD3 heavy chain (SEQ ID NO: 110), linker |
| | (SEQ ID NO: 67), anti-CD3 light chain (SEQ ID NO: 111), His tag. |
| Diplodocus-3 | SEQ ID NO: 77 |
| | Signal sequence (SEQ ID NO: 109), anti-BCMA scFv (SEQ ID NO: 41), |
| | linker (SEQ ID NO: 66), anti-CD3 heavy chain (SEQ ID NO: 112), linker |
| | (SEQ ID NO: 68), anti-CD3 light chain (SEQ ID NO: 113), His tag. |
| Diplodocus-4 | SEQ ID NO: 78 |
| | Signal sequence (SEQ ID NO: 109), anti-BCMA scFv (SEQ ID NO: 41), |
| | linker (SEQ ID NO: 66), anti-CD3 heavy chain (SEQ ID NO: 114), linker |
| | (SEQ ID NO: 68), anti-CD3 light chain (SEQ ID NO: 115), His tag |
| Diplodocus-5 | SEQ ID NO: 79 |
| | Signal sequence (SEQ ID NO: 109), anti-BCMA scFv (SEQ ID NO: 41), |
| | linker (SEQ ID NO: 66), anti-CD3 heavy chain (SEQ ID NO: 116), linker |
| | (SEQ ID NO: 68), anti-CD3 light chain (SEQ ID NO: 117), His tag |
| Diplodocus-6 | SEQ ID NO: 80 |
| | Signal sequence (SEQ ID NO: 109), anti-BCMA scFv (SEQ ID NO: 41), |
| | linker (SEQ ID NO: 66), anti-CD3 heavy chain (SEQ ID NO: 118), linker |
| | (SEQ ID NO: 68), anti-CD3 light chain (SEQ ID NO: 119), His tag |
| Diplodocus-7 | SEQ ID NO: 81 |
| | Signal sequence (SEQ ID NO: 109), anti-BCMA scFv (SEQ ID NO: 41), |
| | linker (Sequence A), human serum albumin (SEQ ID NO: 74), anti-CD3 |
| | heavy chain (SEQ ID NO: 112), linker (SEQ ID NO: 68), anti-CD3 light |
| | chain (SEQ ID NO: 113), His tag. |
| Diplodocus-8 | SEQ ID NO: 82 |
| | Signal sequence (SEQ ID NO: 109), anti-BCMA scFv (SEQ ID NO: 41), |
| | linker (SEQ ID NO: 66), anti-CD3 heavy chain (SEQ ID NO: 118), linker |
| | (SEQ ID NO: 68), anti-CD3 light chain (SEQ ID NO: 119), linker |
| | (Sequence A), human serum albumin (SEQ ID NO: 74), His tag. |
| Diplodocus-9 | SEQ ID NO: 83 |
| | Signal sequence (SEQ ID NO: 109), anti-BCMA scFv (SEQ ID NO: 41), |
| | linker (SEQ ID NO: 66), anti-CD3 heavy chain (SEQ ID NO: 112), linker |
| | (SEQ ID NO: 67), anti-CD3 light chain (SEQ ID NO: 113), His tag. |
| Diplodocus-11 | SEQ ID NO: 85 |
| | Similar to Diplodocus 3 but with a different linker in anti-BCMA scFv, as |
| | shown in the amino acid sequence. |

Functional studies were conducted to determine which of the above bispecific antibodies would best elicit T cell-mediated antimyeloma cytotoxicity. For each of the above proteins (Diplodocus 1-9 and 11), an mRNA construct was prepared, and activated human CD8+ cells were electroporated with the mRNA. As negative controls, CD8+ cells electroporated without mRNA and cells not electroporated were also prepared. The cells were cultured for 5 days. To assess antimyeloma cytotoxicity, supernatant from each cell culture was combined with 40,000 MM.1S cells and 17,000 activated, untransfected human CD8+ cells. The cells were co-cultured for 12 hours. Cytotoxicity was assessed by flow cytometry, with results as follows:

| Name | Cytotoxicity |
|---|---|
| Diplodocus-1 | 20% |
| Diplodocus-2 | 70% |
| Diplodocus-3 | 18% |
| Diplodocus-4 | 19% |
| Diplodocus-5 | 15% |
| Diplodocus-6 | 91% |
| Diplodocus-7 | 12% |
| Diplodocus-8 | <10% |
| Diplodocus-9 | <10% |
| Diplodocus-11 | <10% |
| No electroporation | <10% |
| Electroporation | 13% |
| without mRNA | |

Thus, in a controlled experiment, the anti-BCMA, anti-CD3 bispecific antibodies Diplodocus 6 and Diplodocus 2 were each associated with dramatic antimyeloma activity, as compared to a panel of other anti-BCMA, anti-CD3 bispecific antibodies. The other anti-BCMA, anti-CD3 bispecific antibodies tested showed additional cytotoxicity as compared to the anti-CD19, anti-CD3 control Diplodocus-1.

### Example 3: Sustained In Vitro Activity from Diplodocus-6

Based on the results of Example 2, further studies were carried out on Diplodocus-6. The stability of the Diplodocus-6 protein was assessed in solution at 37° C. Electroporated CD8+ cells expressing Diplodocus-6 were prepared as described in Example 2. Supernatant was isolated from the cells 48 hours after electroporation and then incubated at 37° C. Aliquots of the supernatant were tested for antimyeloma cytotoxicity, as described in Example 2. Results were as follows:

| Timepoint (days) | Cytotoxicity |
|---|---|
| 0 | 90% |
| 1 | 84% |
| 3 | 83% |
| 5 | 75% |
| 6 | 76% |
| 9 | 39% |
| 14 | 23% |

Thus, the anti-BCMA, anti-CD3 bispecific antibody Diplodocus-6 showed sustained activity, with an apparent half-life of about 7 days in culture at 37° C.

### Example 4: Discovery and Functional Comparison of Novel Multispecific Antibodies

A series of multispecific antibodies was produced, with characteristics as follows:

| | Protein Description (detailed description does not necessarily include all |
|---|---|
| Name | amino acids, e.g., linkers) |
| Trilobite-1 | SEQ ID NO: 88 |
| | Diplodocus-6 based tetravalent antibody comprising anti-BCMA scFv (SEQ |
| | ID NO: 41), anti-CD3 scFv (SEQ ID NOs: 118, 119), anti-BCMA scFv |
| | (SEQ ID NO: 41), anti-CD3 scFv (SEQ ID NOs: 118, 119). |
| Trilobite-2 | SEQ ID NO: 89 |
| | Diplodocus-6 based tetravalent antibody similar to Trilobyte-1 but in |
| | different order: anti-BCMA scFv (SEQ ID NO: 41), anti-BCMA scFv (SEQ |
| | ID NO: 41), anti-CD3 scFv (SEQ ID NOs: 118, 119), anti-CD3 scFv (SEQ |
| | ID NOs: 118, 119). |
| Trilobite-3 | SEQ ID NO: 90 |
| | Similar to Trilobyte-2, except difference in linker |
| Trilobite-4 | SEQ ID NO: 91 |
| | Diplodocus-6 based trivalent antibody comprising anti-BCMA scFv (SEQ |
| | ID NO: 41), anti-CD3 scFv (SEQ ID NOs: 118, 119), anti-BCMA scFv |
| | (SEQ ID NO: 41) |
| Trilobite-5 | SEQ ID NO: 92 |
| | Trivalent antibody comprising anti-BCMA scFv (SEQ ID NO: 41), anti- |
| | CD3 scFv (SEQ ID NOs: 118, 119), anti-BCMA scFv (SEQ ID NO: 39) |
| Trilobite-6 | SEQ ID NO: 93 |
| | Tetravalent antibody comprising anti-BCMA scFv (SEQ ID NO: 41), anti- |
| | BCMA scFv (SEQ ID NO: 39), anti-CD3 scFv (SEQ ID NOs: 118, 119), |
| | anti-CD3 scFv (SEQ ID NOs: 118, 119) |
| Trilobite-7 | SEQ ID NO: 94 |
| | Tetravalent antibody comprising anti-BCMA scFv (akin to SEQ ID NO: 41 |
| | with modified linker), anti-BCMA scFv (SEQ ID NO: 39), anti-CD3 scFv |
| | (SEQ ID NOs: 118, 119), anti-CD3 heavy chain (SEQ ID NOs: 112, 113) |
| Trilobite-14 | SEQ ID NO: 95 |
| | Trivalent trispecific antibody comprising anti-albumin nanobody (SEQ ID |
| | NO: 122), anti-BCMA scFv (SEQ ID NO: 41), anti-CD3 scFv (SEQ ID |
| | NOs: 118, 119) |
| Trilobite-15 | SEQ ID NO: 96 |
| | Trivalent trispecific antibody comprising an anti-BCMA scFv (SEQ ID NO: |
| | 41), anti-albumin nanobody (SEQ ID NO: 122), anti-CD3 scFv (SEQ ID |
| | NOs: 118, 119) |
| Trilobite-16 | SEQ ID NO: 97 |
| | Trivalent trispecific antibody comprising an anti-BCMA scFv (SEQ ID NO: |
| | 41), anti-CD3 scFv (SEQ ID NOs: 118, 119), anti-albumin nanobody (SEQ |
| | ID NO: 122) |
| Trilobite-17 | SEQ ID NO: 98 |
| | Bivalent antibody comprising an anti-BCMA scFv (SEQ ID NO: 41), anti- |
| | CD3 scFv (SEQ ID NOs: 118, 119), human IgG1 Fc (SEQ ID NO: 125) |
| Trilobite-18 | SEQ ID NO: 99 |
| | Trivalent trispecific antibody comprising an anti-BCMA scFv (SEQ ID NO: |
| | 41), anti-CD3 scFv (SEQ ID NOs: 118, 119), humanized rabbit anti- |
| | albumin scFv (SEQ ID NOs: 123, 124) |
| Trilobite-19 | SEQ ID NO: 100 |
| | Trivalent trispecific antibody comprising an anti-BCMA scFv (SEQ ID NO: |
| | 41), humanized rabbit anti-albumin scFv (SEQ ID NOs: 123, 124) , anti- |
| | CD3 scFv (SEQ ID NOs: 118, 119) |

Functional studies were conducted to determine which of the above multispecific antibodies would best elicit T cell-mediated antimyeloma cytotoxicity. For each of the above proteins (Trilobite 1-7, 14-19), an mRNA construct was prepared, and activated human CD8+ cells were electroporated with the mRNA. The cells were cultured for 72 hours. To assess antimyeloma cytotoxicity, supernatant from each cell culture was combined with 40,000 MM.1S cells and 20,000 activated, untransfected human CD8+ cells. The cells were co-cultured for 16 hours. Myeloma cell death was assessed by flow cytometry. Results were as follows:

| Name | Cytotoxicity |
|---|---|
| Trilobite-1 | 93% |
| Trilobite-2 | 38% |
| Trilobite-3 | 41% |
| Trilobite-4 | 91% |
| Trilobite-5 | 96% |
| Trilobite-6 | 76% |
| Trilobite-7 | 90% |
| Trilobite-14 | 20% |
| Trilobite-15 | 20% |
| Trilobite-16 | 12% |
| Trilobite-17 | 26% |
| Trilobite-18 | 12% |
| Trilobite-19 | 23% |

Thus, in a controlled comparison of various anti-BCMA, anti-CD3 antibodies, Trilobite-5 was associated with dramatic antimyeloma activity, as compared to a panel of other anti-BCMA, anti-CD3 bispecific antibodies. Trilobite-1, Trilobite 4, and Trilobite-7 also demonstrated excellent antimyeloma activity.

### Example 5. Humanization of the Anti-CD3 Antibody Portion

To improve the activity and reduce immunogenicity potential of inventive anti-BCMA, anti-CD3 bispecific or multispecific antibodies, the anti-CD3 scFv domains were humanized. To humanize these domains, combinations of various amino acid substitutions were proposed by use of tools such as Abysis (www.abysis.org) or by comparison to human germline sequences, e.g., IGHV1*02-03, IGHJ4*01, IGKV1*37-01, IGHJ2-01). Some variants of the anti-CD3 scFv domain comprise (or alternatively, consist of) the amino acid sequences of SEQ ID NO: 1 to SEQ ID NO 38.

FLAG and His-tagged versions of the aforementioned anti-CD3 scFv variants were produced by expression in 293F cells, followed by Nickel column purification.

Each scFv variant was then tested on a CD3 binding assay by combining 10 μg/mL of the scFv variant with 50,000 (CD3+) T cells in FACS buffer. Samples were incubated for 30 minutes at room temperature, washed twice with FACS buffer, and resuspended in FACS buffer containing anti-FLAG FITC antibody. Samples were incubated for 30 minutes at room temperature, washed twice with FACS buffer, resuspended in FACS buffer and analyzed for binding of the anti-FLAG FITC antibody by flow cytometry. CD3 binding results were as follows:

| Anti-CD3 scFv | CD3 Binding |
|---|---|
| Sequence | (increase in MFI) |
| SEQ ID: 1 (TR66) | 32 |
| SEQ ID: 2 (Var 1) | 20 |
| SEQ ID: 3 (Var 2) | 24 |
| SEQ ID: 4 (Var 3) | 24 |
| SEQ ID: 5 (Var 4) | 27 |
| SEQ ID: 6 (Var 5) | 13 |
| SEQ ID: 7 (Var CDR1) | ~0 |
| SEQ ID: 8 (Var CDR2) | ~0 |

Next, 293F cells were used to produce anti-BCMA, anti-CD3 bispecific antibodies that combined some of the above anti-CD3 scFv variants with the anti-BCMA antibody portion of SEQ ID: 41. Each of these bispecific antibodies was then tested (at 1:10,000 dilution) for antimyeloma cytotoxicity by the methods substantially as described in Example 1. Results were as follows:

| Anti-CD3 scFv | Anti-BCMA scFv | |
|---|---|---|
| Sequence | Sequence | Cytotoxicity |
| SEQ ID: 1 (TR66) | SEQ ID NO: 41 (R′) | 40% |
| SEQ ID: 2 (Var 1) | SEQ ID NO: 41 (R′) | 85% |
| SEQ ID: 4 (Var 3) | SEQ ID NO: 41 (R′) | 32% |
| SEQ ID: 5 (Var 4) | SEQ ID NO: 41 (R′) | 75% |

Thus, although the isolated Var 1 scFv was inferior to the isolated TR66 scFv with respect to CD3 binding, when each of these anti-CD3 scFv was combined with an anti-BCMA antibody portion to form a bispecific antibody, Var 1 conferred more than double the cytotoxicity conferred by TR66. A similar counterintuitive and beneficial pattern was seen for Var 4 compared to TR66.

### Example 6. Further Humanization of the Anti-CD3 Domain

To further improve the activity of inventive anti-BCMA, anti-CD3 bispecific or multispecific antibodies, further modifications were made to the anti-CD3 scFv domains of Example 5 to humanize them further.

The antimyeloma cytotoxicity of these variants was tested, this time at 0.22 μg/mL, by methods substantially as described in Example 5. Results were as follows:

| Anti-CD3 scFv | Anti-BCMA scFv | |
|---|---|---|
| Sequence | Sequence | Cytotoxicity (%) |
| SEQ ID: 12 (Var 10) | SEQ ID NO: 41 (R′) | 60.1 |
| SEQ ID: 15 (Var 13) | SEQ ID NO: 41 (R′) | 57.9 |
| SEQ ID: 16 (Var 14) | SEQ ID NO: 41 (R′) | 24.6 |
| SEQ ID: 17 (Var 15) | SEQ ID NO: 41 (R′) | 26.4 |
| SEQ ID: 28 (Var 26) | SEQ ID NO: 41 (R′) | 60.8 |
| SEQ ID: 29 (Var 27) | SEQ ID NO: 41 (R′) | 71.1 |
| SEQ ID: 30 (Var 28) | SEQ ID NO: 41 (R′) | 65.3 |
| SEQ ID: 31 (Var 29) | SEQ ID NO: 41 (R′) | 63.0 |
| SEQ ID: 32 (Var 30) | SEQ ID NO: 41 (R′) | 68.3 |
| SEQ ID: 33 (Var 31) | SEQ ID NO: 41 (R′) | 65.5 |
| SEQ ID: 34 (Var 32) | SEQ ID NO: 41 (R′) | 67.2 |
| SEQ ID: 35 (Var 33) | SEQ ID NO: 41 (R′) | 68.3 |
| SEQ ID: 36 (Var 34) | SEQ ID NO: 41 (R′) | 65.0 |
| SEQ ID: 37 (Var 35) | SEQ ID NO: 41 (R′) | 67.8 |

Also, humanization scores were determined for the anti-CD3 antibody portions described by SEQ ID: 1 (TR66; pre-humanization), SEQ ID: 2 (Var 1, first round of humanization), and SEQ ID: 33 (Var 31, second round of humanization). Results were as follows:

| | | | Theo | | Perfect | Theo |
|---|---|---|---|---|---|---|
| Construct | | Humanness | max | HSC | 9 mer | max |
| TR66 | H | 709 | 999 | 71% | 0 | 111 |
| TR66 | L | 643 | 882 | 73% | 5 | 98 |
| TR66 | Total | 1352 | 1881 | 72% | 5 | 209 |
| Var 1 | H | 844 | 999 | 84% | 56 | 111 |
| Var 1 | L | 712 | 882 | 81% | 35 | 98 |
| Var 1 | Total | 1556 | 1881 | 83% | 91 | 209 |
| Var 31 | H | 861 | 999 | 86% | 67 | 111 |
| Var 31 | L | 731 | 882 | 83% | 38 | 98 |
| Var 31 | Total | 1592 | 1881 | 85% | 105 | 209 |

As shown in the table, the two rounds of humanization yielded humanized anti-CD3 antibody portions that retained excellent function.

### Example 7: Novel Bispecific Antibodies

A series of bispecific antibodies was produced, with characteristics as follows:

| | Protein Identification and Map (detailed description does not |
|---|---|
| Name | necessarily include all amino acids, e.g., linkers) |
| Pterodactyl-4 | SEQ ID: 129, comprising SEQ ID NO: 40, SEQ ID NO: 1; SEQ ID |
| | NO: 40 |
| Pterodactyl-5 | SEQ ID: 130, comprising SEQ ID NO: 41, SEQ ID NO: 2; SEQ ID |
| | NO: 39 |
| Pterodactyl-6 | SEQ ID: 131, comprising SEQ ID NO: 40, SEQ ID NO: 2; SEQ ID |
| | NO: 40 |
| Pterodactyl-11 | SEQ ID: 132, comprising SEQ ID NO: 39; SEQ ID NO: 33 |
| Pterodactyl-12 | SEQ ID: 133, comprising SEQ ID NO: 41, SEQ ID NO: 33; SEQ ID |
| | NO: 39 |
| Pterodactyl-13 | SEQ ID: 134, comprising SEQ ID NO: 39, SEQ ID NO: 33; SEQ ID |
| | NO: 39 |
| Pterodactyl-15 | SEQ ID: 135, comprising SEQ ID NO: 38, SEQ ID NO: 39, SEQ ID |
| | NO: 33; SEQ ID NO: 39 |
| Pterodactyl-16 | SEQ ID: 136, comprising SEQ ID NO: 39, SEQ ID NO: 2; SEQ ID |
| | NO: 39 |
| Pterodactyl-17 | SEQ ID: 137, comprising SEQ ID NO: 38, SEQ ID NO: 39, SEQ ID |
| | NO: 2; SEQ ID NO: 39 |

To each of the above proteins, a Myc-Flag tag of SEQ ID NO: 141 was included at the C-terminus for purposes of the experiment. Functional studies were conducted to assess antimyeloma cytotoxicity of the above constructs. Proteins were prepared and isolated by expression in 293F cells and purification using anti-Myc tag affinity resin.

In a first experiment, antimyeloma cytotoxicity was assessed by addition of different dilutions of purified protein to a coculture of 30,000 MM.1S-GFP cells and 150,000 fresh Peripheral Blood Mononuclear cells from a normal human donor. The cells were co-cultured for 2 days. Myeloma cell death was assessed by flow cytometry. The cytotoxicity of each protein was assessed over a 4-log concentration range, and an EC₅₀ was determined for each. Cytotoxicity results were as follows:

| Name | EC₅₀ (ng/mL) |
|---|---|
| Pterodactyl-4 | 0.06 |
| Pterodactyl-5 | 0.34 |
| Pterodactyl-6 | 0.04 |
| Pterodactyl-11 | 0.98 |
| Pterodactyl-12 | 0.21 |

In a second experiment, antimyeloma cytotoxicity was assessed by addition of different dilutions of purified protein to a coculture of 30,000 MM.1S-GFP cells and 60,000 fresh Peripheral Blood Mononuclear cells from a normal human donor. The cells were co-cultured for 2 days. Myeloma cell death was assessed by flow cytometry. The cytotoxicity of each protein was assessed over a 4-log concentration range, and an EC₅₀ was determined for each. Cytotoxicity results were as follows:

| Name | EC₅₀ (ng/mL) |
|---|---|
| Pterodactyl-4 | 0.19 |
| Pterodactyl-12 | 0.38 |
| Pterodactyl-13 | 1.54 |
| Pterodactyl-15 | 0.15 |
| Pterodactyl-16 | 0.90 |
| Pterodactyl-17 | 0.17 |

Thus, in a controlled experiment, the anti-BCMA, anti-CD3 bispecific antibodies Pterodactyl-4,-6,-12,-15 and-17 were each associated with dramatic antimyeloma activity, as compared to other anti-BCMA, anti-CD3 bispecific antibodies.

### Example 8: Expression of Functional, Secreted Bispecific Antibodies in MSCs

Some of the aforementioned bispecific antibodies were expressed in MSCs by electroporation with mRNA that encoded the respective bispecific antibody (see Example 7 for protein sequences and maps). Electroporation was performed substantially as described in Example 1. MSCs were incubated for 24 hours, whereupon supernatant was collected from the MSCs. To assess antimyeloma cytotoxicity, supernatants were serially diluted and combined with 30,000 MM.1S-GFP cells and 30,000 normal human peripheral blood mononuclear cells. The cells were co-cultured overnight. Activation of CD3+ T cells was assessed by upregulation of the CD69 activation marker by flow cytometry. The capacity of MSC supernatants containing bispecific antibody proteins to trigger T cell activation was assessed over a 4-log concentration range, and an EC₅₀ was determined for each supernatant. Results were as follows:

| Name | EC₅₀ (dilution of supernatant) |
|---|---|
| Pterodactyl-4 | 0.005 |
| Pterodactyl-12 | 0.018 |
| Pterodactyl-15 | 0.004 |
| Pterodactyl-16 | 0.244 |
| Pterodactyl-17 | 0.004 |

Thus, MSCs were successfully modified to secrete the novel anti-BCMA, anti-CD3 bispecific antibodies and achieve antimyeloma activity in a functional assay.

### Example 9: Identification of Bispecific Antibody Pterodactyl-15 in Supernatant of MSCs Modified to Express Pterodactyl-15

The bispecific antibody Pterodactyl-15, corresponding to amino acid SEQ ID NO: 135 (with the Myc-Flag tag of SEQ ID NO: 141 included at the C-terminus), was expressed in human umbilical MSCs by electroporation with an mRNA encoding Pterodactyl-15. MSCs were incubated for 24 hours, whereupon supernatant was collected from the MSCs. A Western blot of the supernatant with an anti-Myc antibody was run under non-reducing conditions. The Western blot showed one protein, which was of ˜85 kD and corresponded to the expected size of Pterodactyl-15.

### Example 10: Synergistic Activity From Co-Expression of IL-12 and Anti-BCMA, Anti-CD3 Bispecific Antibody in a CXCR4-Expressing Cell

Triceratops MSCs were prepared substantially as described in Example 1 (except a Myc-Flag tag of SEQ ID NO: 141 was included at the C-terminus of the bispecific antibody). For comparison, various lots of Comparator cells were prepared by transfection in the presence or absence of mRNA species, as follows:

| | mRNA Types Transfected (Example 1) | | |
|---|---|---|---|
| | Bispecific Antibody | IL-12 Construct | CXCR4 |
| Triceratops | Yes | Yes | Yes |
| Comparator 2 | No | No | No |
| Comparator 3 | Yes | No | Yes |
| Comparator 4 | No | Yes | Yes |
| Comparator 5 | No | No | Yes |

Triceratops and Comparator 2-5 cells were separately plated in tissue culture at 0.5 million cells per mL of RoosterBio media and allowed to adhere for 3 hours. After attachment, the media was removed and replaced with DMEM with 10% heat-inactivated Fetal Bovine Serum. After ˜16 hours, culture supernatants were collected and evaluated for levels of bispecific antibody and IL-12 protein by ELISA as follows:

| | Bispecific antibody | IL-12 concentration |
|---|---|---|
| Culture Supernatant | concentration (ng/mL) | (ng/mL) |
| Triceratops | 95.3 | 68.3 |
| Comparator 2 | 0 | 0 |
| Comparator 3 | 137.0 | 0 |
| Comparator 4 | 0 | 57.3 |
| Comparator 5 | 0 | 0 |

Supernatants were assessed for antimyeloma cytotoxic activity by methods similar to those of Example 1:100,000 PBMC and 25,000 MM1S-GFP cells were plated in a 96-well U-bottom tissue culture plate at a 4:1 effector: target ratio. Supernatants were serially diluted and added to individual wells of the PBMC/MM1S-GFP coculture. Cocultures were incubated at 37° C. for 2 days. Following incubation, supernatants were collected and analyzed for expression of interferon-gamma by ELISA (R&D Systems), and cytotoxicity against MM1S-GFP was evaluated by staining of dead cells with propidium iodide and evaluation of viable MM1S-GFP cell counts by flow cytometry using a Guava EasyCyte 12HT flow cytometer. The cytotoxicity and Interferon-gamma production at a 1:320 dilution of supernatant for each comparator were as follows:

| | MM1S-GFP Cytotoxicity | Interferon- |
|---|---|---|
| Culture Supernatant | (%) | gamma (ng/mL) |
| Triceratops | 91.6% | 20.5 |
| Comparator 2 | 6.2% | 0 |
| Comparator 3 | 89.1% | 3.6 |
| Comparator 4 | 34% | 1.3 |
| Comparator 5 | 4.7% | 0 |

Thus, Triceratops, and Comparator 3, expressing bispecific antibody produced supernatants capable of eliciting substantial levels of anti-myeloma cytotoxicity. In contrast, interferon-gamma production was substantially higher from PBMC/MM1S cocultures incubated with Triceratops supernatant than with those incubated with Comparator 3 and Comparator 4 supernatant, despite high levels of bispecific antibody protein or IL-12 protein in these respective supernatants. Thus, the combination of bispecific antibody and IL-12 produced by Triceratops leads to synergistic induction of the anti-tumor cytokine Interferon-gamma in the context of Peripheral Blood mononuclear cells and myeloma target cells.

### Example 11: Measurement and Modeling of Bispecific Antibody, IL-12 Fusion, and CXCR4 Protein Production in Triceratops Cells

An in vitro experiment was conducted to evaluate stability and decay of proteins generated by Triceratops MSCs. Triceratops MSCs were prepared substantially as described in Example 1 (except a Myc-Flag tag of SEQ ID NO: 141 was included at the C-terminus of the bispecific antibody), and a His tag was included at the C-terminus of the IL-12 engineered protein.

### Methods

Triceratops cells were thawed and cultured for 6 days in complete ROOSTERBIO® medium on CELLBIND® plates. Samples of culture media were taken at 2, 4, 6 and 24 hours, then daily. Samples of supernatant were taken at each timepoint. From timepoints 0 hour to 24 hour, the cell monolayer was left untouched (cumulative protein production), from day 2 to day 6, the medium was replaced and the monolayer was washed between timepoints (time range-dependent protein production). Levels of the bispecific antibody and IL-12 engineered protein in supernatants were evaluated by specific ELISA. Expression of cell-surface expressed CXCR4 was evaluated by harvesting of cells and flow cytometry. The cumulative kinetics of protein production were fitted to an exponential plateau model.

### Results

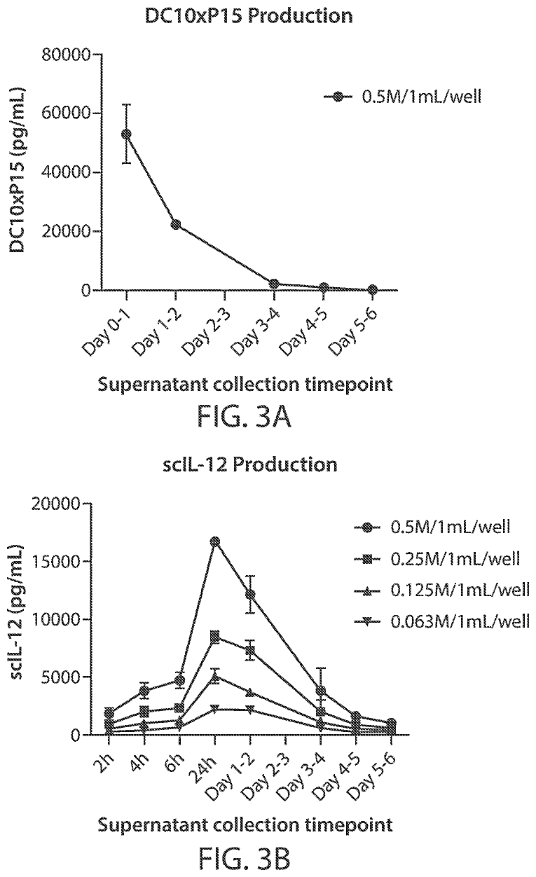

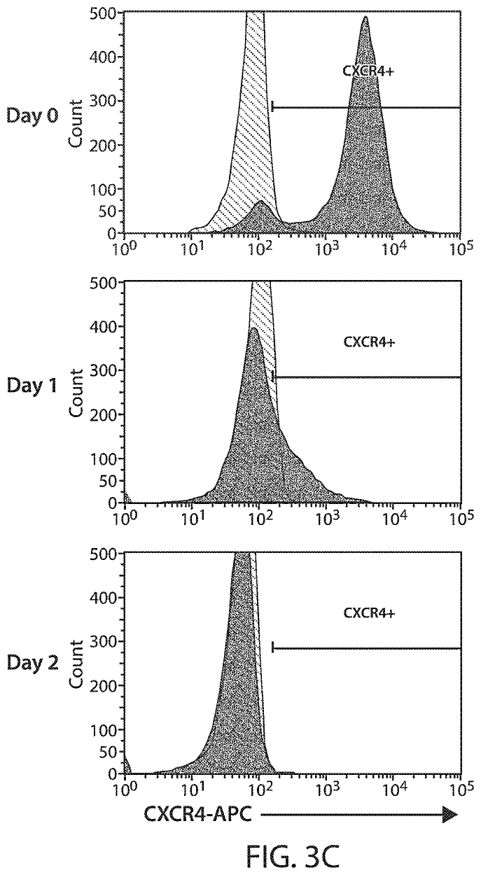

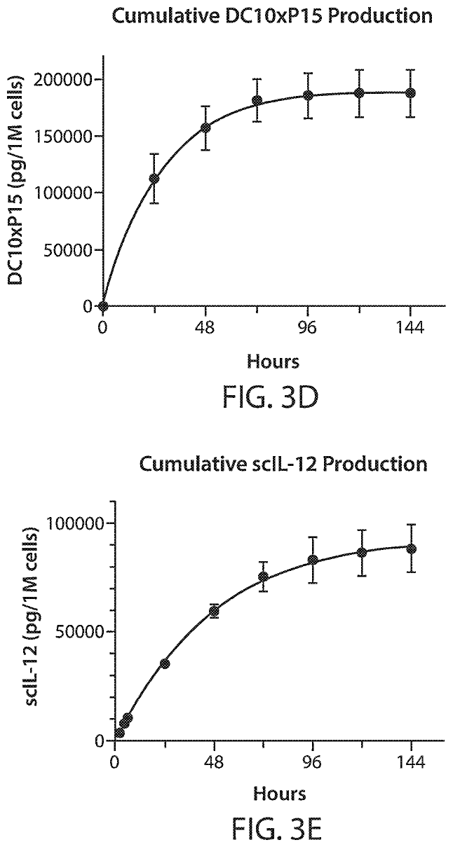

Triceratops MSCs showed high viability (94.2%). Culture produced supernatants with bispecific antibody that was detectable by ELISA through day 4 (FIG. 3A) and day 6, (FIG. 3B) respectively. The quantity of IL-12 engineered protein was cell dose dependent. IL-12 engineered protein quantity increased progressively in cumulative culture conditions up to 24 hours, declining with subsequent 24 hour culture periods (FIG. 3B). CXCR4 expression was detectable of the surface of Triceratops cells, but not control MSC up to day 2 of culture as determined by flow cytometry using a CXCR4-specific antibody (FIG. 3C).

Cumulative bispecific and IL-12 engineered protein production was calculated for 1×106 Triceratops cells and plotted over time (FIGS. 3D and 3E). Exponential plateau curves fit the data with a high goodness of fit (R2=0.92 and 0.98, respectively). Data on maximum quantity of protein produced (pg/1M Triceratops cells) and time rate constant (h−1) are provided in the table immediately below. The calculated maximum bispecific quantity per 106 Triceratops cells was 190 ng with a decay in production rate constant of 0.038/hour. Total maximum calculated production of IL-12 engineered protein quantity was approximately half that of the bispecific protein (94 ng), and IL-12 engineered protein showed a slower decay in production rate (0.021/hour).

| | Maximum quantity | | | |
|---|---|---|---|---|
| | (pg/1M cells) | | K (h⁻¹) | |
| Product | Average | 95% CI | Average | 95% CI |
| Bispecific Ab | 190,370 | 171,956 to 213,353 | 0.03760 | 0.02432 to 0.06115 |
| IL-12 fusion | 94,456 | 86,956 to 104,455 | 0.02100 | 0.01625 to 0.02661 |

### Conclusion

The mRNA-encoded proteins (bispecific, IL-12 engineered protein, and CXCR4) are detected up to day 4, day 6, and day 2, respectively after thaw of Triceratops cells. Modeling of total secreted bispecific antibody and IL-12 engineered protein production provides estimates of maximum production of 190 ng and 94 ng per 1 million Triceratops cells, respectively.

### Example 12: Affinity and Avidity of an Inventive, Anti-BCMA, Anti-CD3 Bispecific Antibody

The binding affinity/avidity of the bispecific antibody of SEQ ID NO: 138 (with a C-terminal Myc-Flag tag of SEQ ID NO:141) for each of its target antigens—BCMA and CD3ε—was determined by Bio-Layer Interferometry.

### Methods

Recombinant protein was generated by transient transfection in Human Embryonic Kidney (HEK) 293 cells using a Expi293® Expression System Kit (Thermo Fisher Scientific) according to the manufacturer's instructions. Expi293F cells were maintained in suspension culture in serum-free chemically-defined medium. On the day of transfection, eukaryotic expression plasmids encoding the protein of interest were complexed with ExpiFectamine293 lipid transfection reagent and added to Expi293F cells at 1 μg per mL of culture. The day after transfection, transfection enhancers were added to the culture. Cultures containing the recombinant protein of interest were collected 2 to 4 days after transfection, centrifuged to remove cell pellets and collect supernatant and filtered through 0.2 to 0.45 μm filters. Protein was either purified from supernatant as described below or supernatant was used directly for analysis. Bispecific antibody protein was purified using the Myc tag. Transfected Expi293F culture supernatants were incubated with anti-c-Myc Agarose (Thermo Scientific, cat. #20168) overnight on a rotator at 4° C. The resin was washed twice with TBS 0.05% Tween-20 and eluted with 1 mg/mL Myc peptide solution (Thermo Scientific, cat. #20170) in TBS. Eluted purified protein was dialyzed in PBS using a 20 kDa cut-off Slide-A-Lyzer Dialysis Cassette (Thermo Scientific, cat. #66003).

The recombinant proteins were quantitated by spectrophotometry. Predicted A280 absorption extinction coefficients and relative molecular masses were determined from analysis of the mature polypeptide sequences using Geneious Prime software (Geneious. Sample concentration was evaluated on a calibrated Nanodrop One (Thermofisher) using a 2 μL volume in triplicate measurements.

Protein purity was evaluated by SDS-PAGE and staining with Coomassie blue dye. Expi293F supernatants or purified proteins were mixed with 4× Laemmli loading buffer (Biorad) (non-reducing) and denatured at 70° C. for 5 minutes. Detatured samples or reference molecular weight markers (Dual Color Protein Marker (Biorad) were loaded on 4-20% Tris-glycine gradient gels (Biorad) and electrophoresis was performed in SDS-glycine running buffer. Following electrophoresis, gels were rinsed and incubated with shaking in Coomassie Brilliant Blue R-250 Staining Solution (Biorad, cat. #1610436) for 1 hour followed by destain in Coomassie Brilliant Blue R-25 Destaining Solution (Biorad, cat. #1610438) overnight.

BCMA and CD3 ligands were purchased from commercial sources and reconstituted according to manufacturers' instructions.

### BCMA and CD3 Ligands for Use in Bio-Layer Interferometry

| | | | Molecular | |
|---|---|---|---|---|
| Protein | Source | Cat/Lot | composition | Structure |
| rhBCMA-Fc | R&D | 193-BC-050/ | Human BCMA (Met1- | Disulfide- |
| Chimera | Systems | EBC1520031 | Ala54) (accession | linked dimer |
| | | | Q6PE46)-IEGRMD- | |
| | | | Human IgG1 (Pro100- | |
| | | | Lys330) | |
| rhCD3ε-Fc | R&D | 9850-CD-050/ | Human CD3ε (Gln22- | Disulfide- |
| Chimera | Systems | DIRS0321021 | Asp126) (accession | linked |
| | | | P07766)-IEGRMD- | homodimer |
| | | | Human IgG1 (Pro100- | |
| | | | Lys330) | |
| Human | ACRO | CDD-H52W0/ | CD3ε (Gln22-Asp126)- | Disulfide- |
| CD3ε&CD3δ | Biosystems | A1160-207TF1- | hIgG1 Fc-His & | linked |
| heterodimeric Fc | | UE | CD3δ (Phe22-Ala105)- | heterodimer |
| Chimera | | | hIgG1 Fc-Flag | |

Ligands (rhBCMA-Fc, rhCD3ε-Fc, or rhCD3ε/CD38-Fc) were captured using an anti-human Fc capture (AHC) dip and read biosensor for 180 s. For association measurement, probes were dipped into wells with the bispecific protein at indicated concentrations in PBS for 340 s to determine the on rate (ka). For dissociation measurement, probes were dipped into assay buffer (PBS) for 600 s to determine the off rate (kd). The KD was determined from the ka and kd using 1:1 local fit analysis (Fortebio software). R² greater than 0.8 was considered acceptable.

Assays were performed at 25 C. The AHC sensor was preconditioned by repeating 3 cycles of dip in regeneration buffer followed by PBS. The sensor was equilibrated in PBS for 10 s. The ligand was captured at the indicated concentration for 30 s on pre-conditioned biosensor. Sensors were then dipped in baseline buffer similar to the antigen diluent (PBS), sensors were then dipped into wells containing antigen at 2-fold serial dilutions as indication. Dissociation was then performed in PBS buffer. Ligand-free parallel reference sensors were run as a control. The association and dissociation constants were determined and used to calculate the KD.

### Results

Raw data were as follows:

### Summary of Scouting for DC10xP15 and DC10xP4 Binding to BCMA, CD3ε and CD3ε/CD3δ Ligands on the Octet

| Loading | Loading | | Conc. | Analyte binding | | Full |
|---|---|---|---|---|---|---|
| Sample ID | response | Analyte | (nM) | Response | KD (M) | R² |
| rhBCMA- | 1.256 | DC10xP15- | 100 | 1.0177 | 1.16E−10 | 0.9932 |
| Fc | | MycDDK | | | | |
| rhCD3ε-Fc | 1.0741 | DC10xP15- | 100 | −0.0165 | NA | 0 |
| | | MycDDK | | | | |
| rhCD3ε-Fc | 1.23 | DC10xP15- | 2000 | −0.0186 | NA | 0 |
| | | MycDDK | | | | |
| rhCD3ε/δ- | 1.4758 | DC10xP15- | 2000 | 0.4959 | 9.76E−09 | 0.8279 |
| Fc | | MycDDK | | | | |

Full kinetic assays were performed for ligands rhBCMA-Fc and rhCD3ε/CD3δ-Fc with the bispecific antibody analyte using concentrations for ligand and analyte determined from Scouting analysis. All six concentrations of the bispecific antibody from 1.56 to 50 nM showed consistent binding to immobilized rhBCMA-Fc with an extremely good fit for the modelled association and dissociation curves (R²=0.9997). The KD was measured at <1.0 pM, with a below-limit-of-detection kd of <1.0×10⁻⁷/s (Table 5). The observed low pM KD measurements were consistent with avidity binding (rather than monovalent affinity binding) to the immobilized BCMA through the bivalent anti-BCMA scFvs in the bispecific antibodies. Therefore, the bispecific antibody showed extremely high avidity for immobilized BCMA.

Four concentrations of bispecific antibody from 15.6 to 125 nM showed consistent binding to immobilized rhCD3ε/δ-Fc with a good fit for the modelled association and dissociation curves (R²=0.9782). The KD was measured at 3.02 nM. Therefore the bispecific antibody showed high affinity for immobilized CD3ε/δ heterodimer.

Summary of Full Kinetic Avidity Measurements for rhBCMA-Fc.

| | | | Concentration | | | | |
|---|---|---|---|---|---|---|---|
| | | Binding | range detected | KD | ka | kd | |
| Ligand | Analyte | mode | (nM) | (M) | (1/Ms) | (1/s) | R² |
| rhBCMA- | DC10xP15- | bivalent | 1.56-50 | <1.0E−12 | 2.26E+05 | <1.0E−07 | 0.9997 |
| Fc | MycDDK | (avidity) | | | | | |

Summary of Full Kinetic Affinity Measurements for rhCD3ε/δ-Fc.

| | | | Concentration | | | | |
|---|---|---|---|---|---|---|---|
| | | Binding | range detected | KD | ka | kd | |
| Ligand | Analyte | mode | (nM) | (M) | (1/Ms) | (1/s) | R² |
| rhCD3ε/δ- | DC10xP15- | monovalent | 15.6-125 | 3.02E−09 | 6.60E+05 | 1.99E−03 | 0.9782 |
| Fc | MycDDK | (affinity) | | | | | |

In summary, the bispecific antibody of SEQ ID NO: 138 (with a C-terminal Myc-Flag tag of SEQ ID NO: 141) displayed very high affinity/avidity for its ligands BCMA (measured avidity KD<1 pM) and high affinity for the naturally occurring form of CD38 (as a heterodimer with either CD3δ or CD3γ; measured affinity KD=3.02 nM).

### Example 12: Off-Target Binding of an Inventive, Anti-BCMA, Anti-CD3 Bispecific Antibody

The off-target binding of the bispecific antibody of SEQ ID NO: 138 (with a C-terminal was assessed.

### Methods

Fc-fusion eukaryotic expression constructs were generated with each of the single-chain Fv molecules in the bispecific antibody. Additional control constructs included an Fc-alone construct and an Fc-fusion of the entire anti-BCMA, anti-CD3 bispecific antibody. Recombinant protein was generated by transient transfection in Human Embryonic Kidney (HEK) 293 cells using a Expi293™ Expression System Kit (Thermo Fisher Scientific) according to the manufacturer's instructions. Expi293F cells were maintained in suspension culture in serum-free chemically-defined medium. On the day of transfection, eukaryotic expression plasmids encoding the protein of interest were complexed with ExpiFectamine293 lipid transfection reagent and added to Expi293F cells at 1 μg per mL of culture. The day after transfection, transfection enhancers were added to the culture. Cultures containing the recombinant protein of interest were collected 3 days after transfection, centrifuged to remove cell pellets and collect supernatant and filtered through 0.2 μm filters. Supernatant was used directly for analysis.

A mixture of supernatant and assay buffer combined at a ratio of 1:1:3 was screened for binding against fixed reverse-transfected HEK293 cells in the Retrogenix cell microarray. A total of 36 slides were used, containing duplicate spots of HEK293 transfected with 5835 human plasma membrane proteins and cell surface tethered human secreted proteins and 371 heterodimers. All transfection efficiencies exceeded the minimum. Following incubation with the FcFusion protein cocktail, detection of FcFusion binding was performed using an AlexaFluor647 anti-mouse IgG H+L detection antibody. Vectors encoding hits identified in the Library Screen plus test and control vectors for CD3ε, CD86 and EGFR were spotted in duplicate on new slides and used to reverse transfect human HEK293 cells as previous. All transfection efficiencies exceeded the minimum threshold.

Transfected HEK293 on Confirmation/Specificity Screen slides were tested after fixation or in the absence of fixation with the combination of Fcfusion-zy307 (anti-BCMA domain-Fc) and Fcfusion-zy308 (anti-CD3 domain-Fc) and assay buffer combined at a ratio of 1:1:3, as used in the Library Screen (1 μg/mL each), along with the individual Fcfusion supernatants at 1 μg/mL and additional control supernatants (Control Fc (zy306) at ˜6 μg/mL, DC10xP15-Fc (zy309) at ˜0.6 μg/mL, Retrogenix positive control CTLA4-mFc at 0.2 μg/mL, or no test article). Slides were analyzed as described above for the Library Screen.

### Results

Strong specific binding was observed for Fcfusion-zy307 (anti-BCMA domain-Fc) to TNFRSF17 (BCMA) transfectants and for Fcfusion-zy308 (anti-CD3 domain-Fc) to cells expressing heterodimers of CD38, confirming the validity of the screen. No binding of Fcfusion-zy308 was observed to the single-gene CD3E transfectant. Both Fcfusion-zy307 and Fcfusion-zy308 test supernatants showed medium binding to cells expressing FcγR2A at a level that was higher than controls. This intermediate-signal interaction was selective but not specific for either Fcfusions and was not observed with Fcfusion-zy309 (entire bispecific antibody-Fc) supernatant, suggesting that the observation was related to the higher concentration of Fcfusion-zy307 and Fcfusion-zy308 (˜1 μg/mL) compared with ˜0.6 μg/mL for Fcfusion-zy309 (entire bispecific antibody-Fc) and the control CTLA4-mFc (0.2 μg/mL) used in the assay. Very weak binding that showed specificity to the Fcfusion-zy308 (anti-CD3 Var31-Fc) was observed in the fixed cell Confirmation for HEK293 transfected with GAD2, HQP3 and HMGB1.

Fcfusion-zy307 (anti-BCMA domain-Fc) and Fcfusion-zy308 (anti-CD3 domain-Fc), in combination, specifically interacted with TNFRSF17 (BCMA) on both fixed and live cells and with CD3E when expressed as part of a heterodimer with CD3G or CD3D on fixed cells and very weakly as part of a heterodimer with CD3D on live cells.

Fcfusion-zy307 alone specifically interacted with TNFRSF17, the primary target, on both fixed and live cells.

Fcfusion-zy308 alone, specifically interacted with CD3E, the primary target, when expressed as part of a heterodimer with CD3G and CD3D on fixed cells and very weakly as part of a heterodimer with CD3D on live cells.

Fcfusion-zy309 (entire bispecific antibody-Fc) specifically interacted with its primary targets TNFRSF17 (BCMA) on both fixed and live cells and with CD3E, when expressed as part of a heterodimer with CD3G or CD3D, on fixed cells, and very weakly as part of a heterodimer with CD3D on live cells.

### Conclusion

The anti-BCMA, anti-CD3 bispecific antibody, as well as its separate anti-BCMA and anti-CD3 domains, show no meaning off-target binding (cross-reactivity) in a human protein screening library.

### Example 13: Antitumor Activity of Serum Collected from a Human Treated with Triceratops Cells

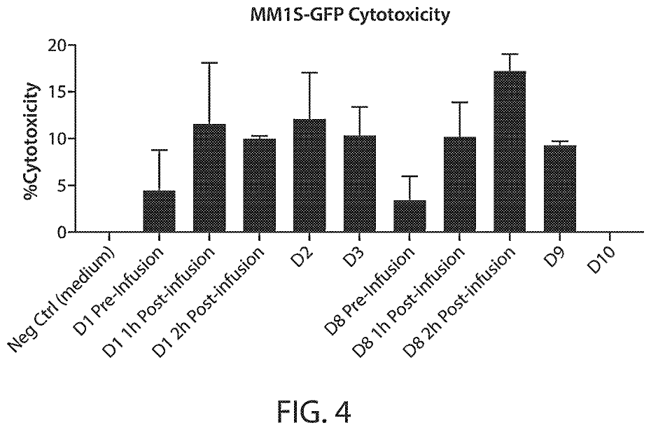

Triceratops cells were administered intravenously, on Days 1 and 8, to a patient with multiple myeloma. Blood was collected and serum isolated from the patient at the following timepoints: Day 1 prior to administration (D1 Pre-Infusion), 1 hour thereafter (D1 1 h Post-Infusion), 2 hours thereafter (D1 2 h Post-Infusion), Day 2 (D2), Day 3 (D3), Day 8 prior to administration (D8 Pre-Infusion), 1 hour thereafter (D8 1 h Post-Infusion), 2 hours thereafter (D8 2 h Post-Infusion), Day 9 (D9), and Day 10 (D10). Cytotoxicity of the heat-inactivated serum samples and negative controls (cell medium) was assessed (by propidium iodide/flow cytometry) following overnight incubation at 37° C. in the presence of 50,000 activated CD8+ T cells and 100,000 MMS1S-GFP cells. The results are shown in FIG. 4. Serum prior to Triceratops administration showed a modest amount of endogenous cytotoxic activity. Following Triceratops administration, there was a marked, reproducible increase in cytotoxic activity. Thus, in a clinical experiment, Triceratops cells (i.e., therapeutic MSCs engineered with RNA to produce an anti-BCMA, anti-CD3 bispecific antibody, IL-12, and CXCR4) conferred marked antitumor/antimyeloma activity in vivo in a patient, as measure in patient serum samples.

### EQUIVALENTS AND SCOPE

Those skilled in the art will recognize or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims, embodiments, or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which two or more members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an Claim, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular Claim of the present invention may be explicitly excluded from any one or more of the claims. Where ranges are given, any value within the range may explicitly be excluded from any one or more of the claims. Any Claim, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

What is claimed is:

1. A mammalian cell comprising:
an exogenous mRNA encoding an anti-BCMA, anti-CD3 antibody; and
an exogenous mRNA encoding a single-chain IL-12 fusion protein.

2. The cell of claim 1, further comprising an exogenous mRNA encoding CXCR4.

3. The cell of either claim 1 or claim 2, wherein the cell is a mesenchymal stem cell.

4. The mesenchymal stem cell of claim 3, wherein the anti-BCMA, anti-CD3 antibody comprises a sequence selected from the group consisting of SEQ ID NOs: 1-64.

5. The mesenchymal stem cell of claim 3, wherein the anti-BCMA, anti-CD3 antibody comprises a sequence selected from the group consisting of SEQ ID NOs: 75-101.


SEQUENCE LISTING (141 sequences)

SEQ ID NO: 1 - protein, 245 aa, synthetic construct
QVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 2 - protein, 245 aa, synthetic construct
QVQLVQSGAEVKKPGASVKVSCKASGYTFTRYTMHWVRQAPGQGLEWIGYINPSKGYTNYNQKFKGRVTMTADKSTSTAYMELSSLRSEDTAVYYCARYYDDHYSLDYWGQGTLVTVSSGGGGSGGGGSGGGGSGGGGSEIVMTQSPATLSLSPGERVTLSCRASSSVSYMNWYQQKSGTSPKRWIYDTSKVASGVPYRFSGSGSGTDFTLTISSLQAEDEATYYCQQWSSNPLTFGQGTKLEIK

SEQ ID NO: 3 - protein, 245 aa, synthetic construct
QVQLVQSGAEVKKPGASVKVSCKASGYTFTRYTMHWVRQAPGQGLEWIGYINPSRGYTNYNQKFKGRVTMTADKSTSTAYMELSSLRSEDTAVYYCARYYDDHYSLDYWGQGTTVTVSSGGGGSGGGGSGGGGSGGGGSEIVMTQSPATLSLSPGERVTLSCRASSSVSYMNWYQQKSGTSPKRWIYDTSKVASGVPYRFSGSGSGTDFTLTISSLQAEDEATYYCQQWSSNPLTFGAGTKLEIK

SEQ ID NO: 4 - protein, 245 aa, synthetic construct
QVQLVQSGAEVKKLGASVKVSCKASGYTFTRYTMHWVKQAPGQGLEWIGYINPSRGYTNYNQKFQGRVTMTRDTSISTAYMELSRLRSDDTAVYYCARYYDDHYSLDYWGQGTLVTVSSGGGGSGGGGSGGGGSGGGGSDIQMTQSPSSLSASVGDRVTITCRASSSVSYMNWYQQKSGTSPKRWIYDTSKVASGVPYRFSGSGSGTDFTLTISSLQPEDFATYYCQQWSSNPLTFGAGTKLEIK

SEQ ID NO: 5 - protein, 245 aa, synthetic construct
QVQLVQSGAEVKKPGASVKVSCKASGYTFTRYTMHWVKQRPGQGLEWIGYINPSKGYTNYNQKFKGRVTMTADKSTSTAYMELSSLRSEDTAVYYCARYYDDHYSLDYWGQGTLVTVSSGGGGSGGGGSGGGGSGGGGSQIVLTQSPAIMSASPGEKVTMTCRASSSVSYMNWYQQKSGTSPKRWIYDTSKVASGVPYRFSGSGSGTDFTLTISSLQAEDEATYYCQQWSSNPLTFGQGTKLEIK

SEQ ID NO: 6 - protein, 245 aa, synthetic construct
QVQLVQSGAEVKKPGASVKVSCKASGYTFTRYTMHWVRQAPGQGLEWIGYINPSRGYTNYNQKFKDKATLTTDKSSSTAYMQLSSLTSEDSAVYYCARYYDDHYSLDYWGQGTLVTVSSGGGGSGGGGSGGGGSGGGGSEIVMTQSPATLSLSPGERVTLSCRASSSVSYMNWYQQKSGTSPKRWIYDTSKVASGVPYRFSGSGSGTSYSLTISSMEAEDAATYYCQQWSSNPLTFGQGTKLEIK

SEQ ID NO: 7 - protein, 246 aa, synthetic construct
QVQLQQSGAELARPGASVKMSCKTSGYTFTGYYMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASQSISSYLNWYQQKSGTSPKRWIYDTSKVASGVPYRFSGSGSGTSYSLTISSMEAEDAATYYCQQWSSNPLTFGAGTKLELK

SEQ ID NO: 8 - protein, 245 aa, synthetic construct
QVQLQQSGAELARPGASVKMSCKTSGYTFTRYTMHWVKQRPGQGLEWMGIINPSGGSTSYAQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGQAPRLLIYGASTRATGIPYRFSGSGSGTSYSLTISSMEAEDAATYYCQQWSSNPLTFGAGTKLELK

SEQ ID NO: 9 - protein, 245 aa, synthetic construct
QVQLVQSGAEVKKPGASVKVSCKASGYTFTRYTMHWVKQRPGQGLEWIGYINPSKGYTNYNQKFKGRVTMTADKSTSTAYMELSSLRSEDTAVYYCARYYDDHYSLDYWGQGTLVTVSSGGGGSGGGGSGGGGSGGGGSEIVMTQSPATLSLSPGERVTLSCRASSSVSYMNWYQQKSGTSPKRWIYDTSKVASGVPYRFSGSGSGTDFTLTISSLQAEDEATYYCQQWSSNPLTFGQGTKLEIK

SEQ ID NO: 10 - protein, 245 aa, synthetic construct
QVQLVQSGAEVKKPGASVKVSCKASGYTFTRYTMHWVRQAPGQGLEWIGYINPSKGYTNYNQKFKGRVTMTADKSTSTAYMELSSLRSEDTAVYYCARYYDDHYSLDYWGQGTLVTVSSGGGGSGGGGSGGGGSGGGGSEIVMTQSPAIMSASPGERVTLSCRASSSVSYMNWYQQKSGTSPKRWIYDTSKVASGVPYRFSGSGSGTDFTLTISSLQAEDEATYYCQQWSSNPLTFGQGTKLEIK

SEQ ID NO: 11 - protein, 245 aa, synthetic construct
QVQLVQSGAEVKKPGASVKVSCKASGYTFTRYTMHWVRQAPGQGLEWIGYINPSKGYTNYNQKFKGRVTMTADKSTSTAYMELSSLRSEDTAVYYCARYYDDHYSLDYWGQGTLVTVSSGGGGSGGGGSGGGGSGGGGSQIVLTQSPAIMSASPGEKVTMTCRASSSVSYMNWYQQKSGTSPKRWIYDTSKVASGVPYRFSGSGSGTDFTLTISSLQAEDEATYYCQQWSSNPLTFGQGTKLEIK

SEQ ID NO: 12 - protein, 245 aa, synthetic construct
QVQLVQSGAEVKKPGASVKVSCKASGYTFTRYTMHWVRQAPGQGLEWIGYINPSKGYTNYNQKFKGRVTMTADKSTSTAYMELSSLRSEDTAVYYCARYYDDHYSLDYWGQGTLVTVSSGGGGSGGGGSGGGGSGGGGSEIVMTQSPATLSVSPGERATLSCRASSSVSYLNWYQQKSGTSPKRWIYDTSKVASGVPYRFSGSGSGTDFTLTISSLQAEDEATYYCQQWSSNPLTFGQGTKLEIK

SEQ ID NO: 13 - protein, 245 aa, synthetic construct
QVQLVQSGAEVKKPGASVKVSCKASGYTFTRYTMHWVRQAPGQGLEWIGYINPSKGYTNYNQKFKGRVTMTADKSTSTAYMELSSLRSEDTAVYYCARYYDDHYSLDYWGQGTLVTVSSGGGGSGGGGSGGGGSGGGGSEIVLTQSPATLSLSPGERATLSCRASSSVSYLNWYQQKSGTSPRRWIYDTSKVATGIPYRFSGSGSGTDFTLTISSLEAEDEATYYCQQWSSNPLTFGQGTKLEIK

SEQ ID NO: 14 - protein, 245 aa, synthetic construct
QVQLVQSGAEVKKPGASVKVSCKASGYTFTRYTMHWVRQAPGQGLEWIGYINPSKGYTNYNQKFKGRVTMTADKSTSTAYMELSSLRSEDTAVYYCARYYDDHYSLDYWGQGTLVTVSSGGGGSGGGGSGGGGSGGGGSEIVLTQSPATLSLSPGERVTLSCRASSSVSYLNWYQQKSGTSPKRWIYDTSKVATGIPYRFSGSGSGTDFTLTISSLQAEDEATYYCQQWSSNPLTFGQGTKLEIK

SEQ ID NO: 15 - protein, 245 aa, synthetic construct
QVQLVQSGAEVKKPGASVKVSCKASGYTFTRYTMHWVRQAPGQGLEWMGYINPSKGYTNYNQKFKGRVTMTADKSTSTVYMELSSLRSEDTAVYYCARYYDDHYSLDYWGQGTLVTVSSGGGGSGGGGSGGGGSGGGGSEIVMTQSPATLSLSPGERVTLSCRASSSVSYMNWYQQKSGTSPKRWIYDTSKVASGVPYRFSGSGSGTDFTLTISSLQAEDEATYYCQQWSSNPLTFGQGTKLEIK

SEQ ID NO: 16 - protein, 245 aa, synthetic construct
QVQLVQSGAEVKKPGASVKVSCKASGYTFTRYTMHWVRQAPGQGLEWMGYINPSGGSTSYAQKFKGRVTMTADKSTSTVYMELSSLRSEDTAVYYCARYYDDHYSLDYWGQGTLVTVSSGGGGSGGGGSGGGGSGGGGSEIVMTQSPATLSLSPGERVTLSCRASSSVSYMNWYQQKSGTSPKRWIYDTSKVASGVPYRFSGSGSGTDFTLTISSLQAEDEATYYCQQWSSNPLTFGQGTKLEIK

SEQ ID NO: 17 - protein, 245 aa, synthetic construct
QVQLVQSGAEVKKPGASVKVSCKASGYTFTSYYMHWVRQAPGQGLEWMGYINPSKGYTNYNQKFKGRVTMTADKSTSTVYMELSSLRSEDTAVYYCARYYDDHYSLDYWGQGTLVTVSSGGGGSGGGGSGGGGSGGGGSEIVMTQSPATLSLSPGERVTLSCRASSSVSYMNWYQQKSGTSPKRWIYDTSKVASGVPYRFSGSGSGTDFTLTISSLQAEDEATYYCQQWSSNPLTFGQGTKLEIK

SEQ ID NO: 18 - protein, 245 aa, synthetic construct
QVQLVQSGAEVKKPGASVKVSCKASGYTFTRYTMHWVRQAPGQGLEWIGYINPSKGYTNYNQKFKGRVTMTADKSTSTAYMELSSLRSEDTAVYYCARYYDDHYSLDYWGQGTLVTVSSGGGGSGGGGSGGGGSGGGGSEIVLTQSPATLSLSPGERVTLSCRASSSVSYLNWYQQKSGTSPKRWIYDASKVATGIPYRFSGSGSGTDFTLTISSLQAEDEATYYCQQRSNWPLTFGQGTKLEIK

SEQ ID NO: 19 - protein, 245 aa, synthetic construct
QVQLVQSGAEVKKPGASVKVSCKASGYTFTRYTMHWVRQAPGQGLEWMGYINPSKGYTNYNQKFKGRVTMTADKSTSTVYMELSSLRSEDTAVYYCARYYDDHYSLDYWGQGTLVTVSSGGGGSGGGGSGGGGSGGGGSEIVMTQSPAIMSASPGERVTLSCRASSSVSYMNWYQQKSGTSPKRWIYDTSKVASGVPYRFSGSGSGTDFTLTISSLQAEDEATYYCQQWSSNPLTFGQGTKLEIK

SEQ ID NO: 20 - protein, 245 aa, synthetic construct
QVQLVQSGAEVKKPGASVKVSCKASGYTFTSYYMHWVRQAPGQGLEWMGYINPSKGYTNYNQKFKGRVTMTADKSTSTVYMELSSLRSEDTAVYYCARYYDDHYSLDYWGQGTLVTVSSGGGGSGGGGSGGGGSGGGGSEIVMTQSPAIMSASPGERVTLSCRASSSVSYMNWYQQKSGTSPKRWIYDTSKVASGVPYRFSGSGSGTDFTLTISSLQAEDEATYYCQQWSSNPLTFGQGTKLEIK

SEQ ID NO: 21 - protein, 245 aa, synthetic construct
QVQLVQSGAEVKKPGASVKVSCKASGYTFTRYTMHWVKQRPGQGLEWIGYINPSKGYTNYNQKFKGRVTMTADKSTSTAYMELSSLRSEDTAVYYCARYYDDHYSLDYWGQGTLVTVSSGGGGSGGGGSGGGGSGGGGSEIVLTQSPATLSLSPGERVTLSCRASSSVSYLNWYQQKSGTSPKRWIYDTSKVATGIPYRFSGSGSGTDFTLTISSLQAEDEATYYCQQWSSNPLTFGQGTKLEIK

SEQ ID NO: 22 - protein, 245 aa, synthetic construct
QVQLVQSGAEVKKPGASVKVSCKASGYTFTRYTMHWVKQRPGQGLEWIGYINPSKGYTNYNQKFKGRVTMTADKSTSTAYMELSSLRSEDTAVYYCARYYDDHYSLDYWGQGTLVTVSSGGGGSGGGGSGGGGSGGGGSEIVLTQSPATLSLSPGERVTLSCRASSSVSYLNWYQQKSGTSPKRWIYDASKVATGIPYRFSGSGSGTDFTLTISSLQAEDEATYYCQQRSNWPLTFGQGTKLEIK

SEQ ID NO: 23 - protein, 245 aa, synthetic construct
QVQLVQSGAEVKKPGASVKVSCKASGYTFTSYYMHWVRQAPGQGLEWMGYINPSKGYTNYNQKFKGRVTMTADKSTSTVYMELSSLRSEDTAVYYCARYYDDHYSLDYWGQGTLVTVSSGGGGSGGGGSGGGGSGGGGSEIVLTQSPATLSLSPGERVTLSCRASSSVSYLNWYQQKSGTSPKRWIYDASKVATGIPYRFSGSGSGTDFTLTISSLQAEDEATYYCQQRSNWPLTFGQGTKLEIK

SEQ ID NO: 24 - protein, 245 aa, synthetic construct
QVQLVQSGAEVKKLGASVKVSCKASGYTFTRYTMHWVKQAPGQGLEWMGYINPSRGYTNYNQKFQGRVTMTRDTSISTAYMELSRLRSDDTAVYYCARYYDDHYSLDYWGQGTLVTVSSGGGGSGGGGSGGGGSGGGGSDIQMTQSPSSLSASVGDRVTITCRASSSISYLNWYQQKSGTSPKRWIYDTSKVASGVPYRFSGSGSGTDFTLTISSLQPEDFATYYCQQWSSNPLTFGAGTKLEIK

SEQ ID NO: 25 - protein, 245 aa, synthetic construct
QVQLVQSGAEVKKPGASVKVSCKASGYTFTRYTMHWVRQRPGQGLEWMGYINPSRGYTNYNQKFQGRVTMTRTTSSSTAYMELSSLRSDDTAVYYCARYYDDHYSLDYWGQGTLVTVSSGGGGSGGGGSGGGGSGGGGSDIQMTQSPSSLSASVGDRVTITCRASSSVSYMNWYQQKSGTSPKRWIYDTSKVASGVPYRFSGSGSGTDFTLTISSLQPEDFATYYCQQWSSNPLTFGAGTKLEIK

SEQ ID NO: 26 - protein, 245 aa, synthetic construct
QVQLVQSGAEVKKLGASVKVSCKASGYTFTRYTMHWVKQAPGQGLEWIGYINPSRGYTNYNQKFQGRVTMTRDTSISTAYMELSRLRSDDTAVYYCARYYDDHYSLDYWGQGTLVTVSSGGGGSGGGGSGGGGSGGGGSDIQMTQSPSSLSASVGDRVTITCRASSSVSYMNWYQQKSGTSPKRWIYDTSKVASGVPYRFSGSGSGTDFTLTISSLQAEDAATYYCQQWSSNPLTFGAGTKLEIK

SEQ ID NO: 27 - protein, 245 aa, synthetic construct
QVQLVQSGAEVKKPGASVKVSCKASGYTFTRYTMHWVRQRPGQGLEWMGYINPSRGYTNYNQKFQGRVTMTRTTSSSTAYMELSSLRSDDTAVYYCARYYDDHYSLDYWGQGTLVTVSSGGGGSGGGGSGGGGSGGGGSDIQMTQSPSSLSASVGDRVTITCRASSSVSYMNWYQQKSGTSPKRWIYDTSKVASGVPYRFSGSGSGTDFTLTISSLQAEDAATYYCQQWSSNPLTFGAGTKLEIK

SEQ ID NO: 28 - protein, 245 aa, synthetic construct
QVQLVQSGAEVKKPGASVKVSCKASGYTFTRYTMHWVRQAPGQGLEWMGYINPSKGYTNYNQKFKGRVTMTADKSTSTAYMELSSLRSEDTAVYYCARYYDDHYSLDYWGQGTLVTVSSGGGGSGGGGSGGGGSGGGGSEIVMTQSPATLSVSPGERATLSCRASSSVSYLNWYQQKSGTSPKRWIYDTSKVASGVPYRFSGSGSGTDFTLTISSLQAEDEATYYCQQWSSNPLTFGQGTKLEIK

SEQ ID NO: 29 - protein, 245 aa, synthetic construct
QVQLVQSGAEVKKPGASVKVSCKASGYTFTRYTMHWVRQAPGQGLEWMGYINPSKGYTNYNQKFKGRVTITADKSTSTAYMELSSLRSEDTAVYYCARYYDDHYSLDYWGQGTLVTVSSGGGGSGGGGSGGGGSGGGGSEIVMTQSPATLSVSPGERATLSCRASSSVSYLNWYQQKSGTSPKRWIYDTSKVASGVPYRFSGSGSGTDFTLTISSLQAEDEATYYCQQWSSNPLTFGQGTKLEIK

SEQ ID NO: 30 - protein, 245 aa, synthetic construct
QVQLVQSGAEVKKPGASVKVSCKASGYTFTRYTMHWVRQAPGQGLEWMGYINPSKGYTNYNQKFKGRVTMTADKSTSTAYMELSSLRSEDTAVYYCARYYDDHYSLDYWGQGTLVTVSSGGGGSGGGGSGGGGSGGGGSEIVMTQSPATLSVSPGERATLSCRASSSVSYLNWYQQKPGTAPKRWIYDTSKVASGVPYRFSGSGSGTDFTLTISSLQAEDEATYYCQQWSSNPLTFGQGTKLEIK

SEQ ID NO: 31 - protein, 245 aa, synthetic construct
QVQLVQSGAEVKKPGASVKVSCKASGYTFTRYTMHWVRQAPGQGLEWMGYINPSKGYTNYNQKFKGRVTMTADKSTSTAYMELSSLRSEDTAVYYCARYYDDHYSLDYWGQGTLVTVSSGGGGSGGGGSGGGGSGGGGSEIVMTQSPATLSVSPGERATLSCRASSSVSYLNWYQQKSGTSPKRWIYDTSKLESGVPYRFSGSGSGTDFTLTISSLQSEDEATYYCQQWSSNPLTFGQGTKLEIK

SEQ ID NO: 32 - protein, 245 aa, synthetic construct
QVQLVQSGAEVKKPGASVKVSCKASGYTFTRYTMHWVRQAPGQGLEWMGYINPSKGYTNYNQKFKGRVTMTADKSTSTAYMELSSLRSEDTAVYYCARYYDDHYSLDYWGQGTLVTVSSGGGGSGGGGSGGGGSGGGGSEIVMTQSPATLSVSPGERATLSCRASSSVSYLNWYQQKPGSAPKRLIYDTSKLESGVPYRFSGSGSGTDFTLTISSLQSEDEADYYCQQWSSNPLTFGQGTKLEIK

SEQ ID NO: 33 - protein, 245 aa, synthetic construct
QVQLVQSGAEVKKPGASVKVSCKASGYTFTRYTMHWVRQAPGQGLEWMGYINPSKGYTNYNQKFKGRVTITADKSTSTAYMELSSLRSEDTAVYYCARYYDDHYSLDYWGQGTLVTVSSGGGGSGGGGSGGGGSGGGGSEIVMTQSPATLSVSPGERATLSCRASSSVSYLNWYQQKPGTAPKRWIYDTSKVASGVPYRFSGSGSGTDFTLTISSLQAEDEATYYCQQWSSNPLTFGQGTKLEIK

SEQ ID NO: 34 - protein, 245 aa, synthetic construct
QVQLVQSGAEVKKPGASVKVSCKASGYTFTRYTMHWVRQAPGQGLEWMGYINPSKGYTNYNQKFKGRVTITADKSTSTAYMELSSLRSEDTAVYYCARYYDDHYSLDYWGQGTLVTVSSGGGGSGGGGSGGGGSGGGGSEIVMTQSPATLSVSPGERATLSCRASSSVSYLNWYQQKSGTSPKRWIYDTSKLESGVPYRFSGSGSGTDFTLTISSLQSEDEATYYCQQWSSNPLTFGQGTKLEIK

SEQ ID NO: 35 - protein, 245 aa, synthetic construct
QVQLVQSGAEVKKPGASVKVSCKASGYTFTRYTMHWVRQAPGQGLEWMGYINPSKGYTNYNQKFKGRVTITADKSTSTAYMELSSLRSEDTAVYYCARYYDDHYSLDYWGQGTLVTVSSGGGGSGGGGSGGGGSGGGGSEIVMTQSPATLSVSPGERATLSCRASSSVSYLNWYQQKPGTAPKRWIYDTSKLASGVPYRFSGSGSGTDFTLTISSLQSEDEATYYCQQWSSNPLTFGQGTKLEIK

SEQ ID NO: 36 - protein, 245 aa, synthetic construct
QVQLVQSGAEVKKPGASVKVSCKASGYTFTRYTMHWVRQAPGQGLEWMGYINPSKGYTNYNQKFKGRVTITADKSTSTAYMELSSLRSEDTAVYYCARYYDDHYSLDYWGQGTLVTVSSGGGGSGGGGSGGGGSGGGGSEIVMTQSPATLSVSPGERATLSCRASSSVSYLNWYQQKPGSAPKRWIYDTSKLESGVPYRFSGSGSGTDFTLTISSLQSEDEATYYCQQWSSNPLTFGQGTKLEIK

SEQ ID NO: 37 - protein, 245 aa, synthetic construct
QVQLVQSGAEVKKPGASVKVSCKASGYTFTRYTMHWVRQAPGQGLEWMGYINPSKGYTNYNQKFKGRVTITADKSTSTAYMELSSLRSEDTAVYYCARYYDDHYSLDYWGQGTLVTVSSGGGGSGGGGSGGGGSGGGGSEIVMTQSPATLSVSPGERATLSCRASSSVSYLNWYQQKPGSAPKRLIYDTSKLESGVPYRFSGSGSGTDFTLTISSLQSEDEADYYCQQWSSNPLTFGQGTKLEIK

SEQ ID NO: 38 - protein, 245 aa, synthetic construct
QVQLVQSGAEVKKPGASVKVSCKASGYTFTRYTMHWVRQAPGQGLEWMGYINPSKGYTNYNQKFKGRVTITADKSTSTAYMELSSLRSEDTAVYYCARYYDDHYSLDYWGQGTLVTVSSGGGGSGGGGSGGGGSGGGGSEIVMTQSPATLSVSPGERATLSCRASSSVSYLNWYQQKSGSAPKRWIYDTSKLESGVPYRFSGSGSGTDFTLTISSLQAEDEADYYCQQWSSNPLTFGQGTKLEIK

SEQ ID NO: 39 - protein, 246 aa, synthetic construct
DIVLTQSPASLAVSPGQRATITCRASESVTILGSHLIHWYQQKPGQPPKLLINLASNVNTGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTDYSINWVRQAPGKGLEWVGWINTETREPAYAYDFTGRFTFSADTSKSMAYLQINSLRAEDTAVYYCALDYTYGMDYWGQGTLVTVSS

SEQ ID NO: 40 - protein, 246 aa, synthetic construct
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 41 - protein, 246 aa, synthetic construct
DIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSS

SEQ ID NO: 42 - protein, 246 aa, synthetic construct
DIVMTQSPDSLSVSLGERATINCRASESVTILGSHLIHWYQQKPGQPPKLLIQLASNVQTGVPDRFSGSGSGTDFTLTISSLQAEDVAVYYCLQSRTIPRTFGGGTKVEIKGSTSGSGKPGSGEGSTKGQIQLVQSGAEVKKPGASVKISCKASGYTFTDYSINWVRQAPGQGLEWMGWINTETREPAYAYDFRGRVTMTRDTSASTAYLQISSLKAEDTAVYFCALDYSYAMDYWGQGSLVTVSS

SEQ ID NO: 43 - protein, 246 aa, synthetic construct
DIVLTQSPASLAVSPGQRATITCRASESVTILGSHLIHWYQQKPGQPPKLLIQLASNVQTGVPARFSGSGSGTDFTLTISSVEPEDTANYYCLQSRTIPRTFGQGTKVEIKGSTSGSGKPGSGEGSTKGQIQLVQSGPELKKPGGSVKISCKASGYTFTDYSINWVRQAPGKGLEWVGWINTETREPAYAYDFTGRFTFSADTSKSMAYLQINSLRAEDTAVYYCALDYSYAMDYWGQGTLVTVSS

SEQ ID NO: 44 - protein, 246 aa, synthetic construct
DIVLTQSPASLAVSLGKRATISCRASESVTILGSHLIHWYQQKPGQPPKLLIYLASNVQTGVPARFSGSGSGTDFTLTISSLEAEDVAVYYCLQSRTIPRTFGQGTKLEIKGSTSGSGKPGSGEGSTKGQVQLVQSGAEVKKPGASVKISCKASGYTFTDYSINWVRQAPGQGLEWMGWINTETREPAYAYDFRGRVTMTRDTSASTAYLQISSLKAEDTAVYFCALDYSYAMDYWGQGSLVTVSS

SEQ ID NO: 45 - protein, 246 aa, synthetic construct
DIVMTQSPDSLSVSLGERATINCRASESVTILGSHLIHWYQQKPGQPPKLLIQLASNVQTGVPDRFSGSGSGTDFTLTISSLQAEDVAVYYCLQSRTIPRTFGGGTKVEIKGSTSGSGKPGSGEGSTKGQIQLVQSGAEVKKPGESVKISCKASGYTFTDYSINWVRQAPGQGLKWMGWINTETREPAYAYDFRGRFAFSLDTSASTAYLQISSLKAEDTAVYFCALDYSYAMDYWGQGTLVTVSS

SEQ ID NO: 46 - protein, 246 aa, synthetic construct
DIVLTQSPASLAVSLGERATISCRASESVSILGSHYLAWYQQKPGQPPKLLIYLASNLQTGVPARFSGSGSGTDFTLTISSLEAEDVAVYYCQQSRTIPRTFGQGTKLEIKGSTSGSGKPGSGEGSTKGQIQLVQSGAEVKKPGESVKISCKASGYTFTDYSINWVRQAPGQGLKWMGWINTETREPAYAYDFRGRFAFSLDTSASTAYLQISSLKAEDTAVYFCALDYSYAMDYWGQGTLVTVSS

SEQ ID NO: 47 - protein, 246 aa, synthetic construct
DIVLTQSPSSLSASVGDRATISCRASESVTILGSHLIHWYQQKPGQAPKLLIQLASNVQTGVPARFSGSGSGTDFTLTISSVEPDDVAVYYCLQSRTIPRTFGGGTKLEIKGSTSGSGKPGSGEGSTKGQIQLVQSGPELKKPGGSVKISCAASGYTFTDYSINWVKQAPGKGLEWVGWINTETREPAYAYDFRGRFTFSLDTSKSTAYLQMNSLRAEDTAVYYCALDYSYAMDYWGQGTLVTVSS

SEQ ID NO: 48 - protein, 246 aa, synthetic construct
DIVLTQSPASLAVSPGQRATISCRASESVTILGSHLIHWYQQKPGQPPKLLIQLASNVQTGVPARFSGSGSRTDFTLTISSLEPEDVAVYYCLQSRTIPRTFGQGTKVEIKGSTSGSGKPGSGEGSTKGQITLKESGPTLVKPTQTLTLSCKASGYTFTDYSINWVRRAPGKGLEWMGWINTETREPAYAYDFRGRFVFSLDTSVSMAYLQISSLKAEDTAVYYCALDYSYAMDYWGQGTLVTVSS

SEQ ID NO: 49 - protein, 246 aa, synthetic construct
DIVLTQSPSSLSASVGDRATISCRASESVTILGSHLIHWYQQKPGQAPKLLIQLASNVQTGVPARFSGSGSGTDFTLTISSVEPEDVAVYYCLQSRTIPRTFGQGTKVEIKGSTSGSGKPGSGEGSTKGQIQLVQSGPELKKPGGSVKISCAASGYTFTDYSINWVKQAPGKGLEWVGWINTETREPAYAYDFRGRFTFSADTSKSTAYLQMNSLRAEDTAVYYCALDYSYAMDYWGQGTLVTVSS

SEQ ID NO: 50 - protein, 246 aa, synthetic construct
DIVLTQSPSSLSASVGDRATISCRASESVTILGSHLIHWYQQKPGQAPKLLIQLASNVQTGVPARFSGSGSGTDFTLTISSVEPEDVAVYYCLQSRTIPRTFGQGTKVEIKGSTSGSGKPGSGEGSTKGQIQLVQSGPELKKPGGSVKISCAASGYTFTDYSINWVRQAPGKGLEWVGWINTETREPAYAYDFRGRFTFSADTSKSTAYLQMNSLRAEDTAVYYCALDYSYAMDYWGQGTLVTVSS

SEQ ID NO: 51 - protein, 246 aa, synthetic construct
DIVLTQSPSSLSASVGDRATISCRASESVTILGSHLIHWYQQKPGQAPKLLIQLASNVQTGVPARFSGSGSGTDFTLTISSVEPEDVAVYYCLQSRTIPRTFGQGTKVEIKGSTSGSGKPGSGEGSTKGQIQLVESGAEVKKPGGSVKISCAASGYTFTDYSINWVKQAPGKGLEWVGWINTETREPAYAYDFRGRFTFSLDTSKSTAYLQMNSLRAEDTAVYYCALDYSYAMDYWGQGTLVTVSS

SEQ ID NO: 52 - protein, 246 aa, synthetic construct
DIVLTQSPSSLSASVGDRATISCRASESVTILGSHLIHWYQQKPGQAPKLLIQLASNVQTGVPARFSGSGSGTDFTLTISSVEPEDVAVYYCLQSRTIPRTFGQGTKVEIKGSTSGSGKPGSGEGSTKGQIQLVESGAEVKKPGGSVKVSCKASGYTFTDYSINWVRQAPGKGLEWVGWINTETREPAYAYDFRGRFAISAETSKSTAYLQMNSLRAEDTAVYYCALDYSYAMDYWGQGTLVTVSS

SEQ ID NO: 53 - protein, 246 aa, synthetic construct; identical to SEQ ID NO: 43

SEQ ID NO: 54 - protein, 246 aa, synthetic construct
DIVLTQSPSSLSASVGDRATISCRASESVTILGSHLIHWYQQKPGQAPKLLIQLASNVQTGVPARFSGSGSGTDFTLTISSVEPEDVAVYYCLQSRTIPRTFGQGTKVEIKGSTSGSGKPGSGEGSTKGQIQLVQSGPELKKPGGSVKISCAASGYTFTSYDINWVRQAPGKGLEWVGWINTETREPAYAYDFRGRFTFSADTSKSTAYLQMNSLRAEDTAVYYCALDYSYAMDYWGQGTLVTVSS

SEQ ID NO: 55 - protein, 246 aa, synthetic construct
DIVLTQSPSSLSASVGDRATISCRASESVTILGSHLIHWYQQKPGQAPKLLIQLASNVQTGVPARFSGSGSGTDFTLTISSVEPEDVAVYYCLQSRTIPRTFGQGTKVEIKGSTSGSGKPGSGEGSTKGQIQLVQSGPELKKPGGSVKISCAASGYTFTDYSINWVRQAPGKGLEWVGWINTETREPAYAYDFRGRFTFSADTSKSTAYLQMNSLRAEDTAVYYCALDYTYGMDYWGQGTLVTVSS

SEQ ID NO: 56 - protein, 246 aa, synthetic construct
DIVLTQSPSSLSASVGDRATISCRASESVSFLGINLIHWYQQKPGQAPKLLIYSASNLQSGVPARFSGSGSGTDFTLTISSVEPEDVAVYYCLQSRTLPRTFGQGTKVEIKGSTSGSGKPGSGEGSTKGQIQLVQSGPELKKPGGSVKISCAASGYTFTDYSINWVRQAPGKGLEWVGWINTETREPAYAYDFRGRFTFSADTSKSTAYLQMNSLRAEDTAVYYCALDYSYAMDYWGQGTLVTVSS

SEQ ID NO: 57 - protein, 246 aa, synthetic construct
DIVLTQSPSSLSASVGDRATISCRASESVSFLGINLIHWYQQKPGQAPKLLIYSASNLQSGVPARFSGSGSGTDFTLTISSVEPEDVAVYYCLQSKNFPRTFGQGTKVEIKGSTSGSGKPGSGEGSTKGQIQLVQSGPELKKPGGSVKISCAASGYTFTDYSINWVRQAPGKGLEWVGWINTETREPAYAYDFRGRFTFSADTSKSTAYLQMNSLRAEDTAVYYCALDYSYAMDYWGQGTLVTVSS

SEQ ID NO: 58 - protein, 246 aa, synthetic construct
DIVLTQSPSSLSASVGDRATISCRASESVTILGSHLIHWYQQKPGQAPKLLIQLASNVQTGVPARFSGSGSGTDFTLTISSVEPEDVAVYYCLQSRTIPRTFGQGTKVEIKGSTSGSGKPGSGEGSTKGQIQLVQSGPELKKPGGSVKISCAASGYTFTSYSINWVRQAPGKGLEWVGWINTETREPAYAQGFTGRFTFSADTSKSTAYLQMNSLRAEDTAVYYCALDYLYSLDFWGQGTLVTVSS

SEQ ID NO: 59 - protein, 246 aa, synthetic construct
DIVLTQSPSSLSASVGDRATISCRASESVTILGSHLIHWYQQKPGQAPKLLIQLASNVQTGVPARFSGSGSGTDFTLTISSVEPEDVAVYYCLQSRTIPRTFGQGTKVEIKGSTSGSGKPGSGEGSTKGQIQLVQSGPELKKPGGSVKISCAASGYTFTSYDINWVRQAPGKGLEWVGWINTNTGNPTYAQGFTGRFTFSADTSKSTAYLQMNSLRAEDTAVYYCALDYTYGMDYWGQGTLVTVSS

SEQ ID NO: 60 - protein, 246 aa, synthetic construct
DIVLTQSPSSLSASVGDRATISCRASESVTILGSHLIHWYQQKPGQAPKLLIQLASNVQTGVPARFSGSGSGTDFTLTISSVEPEDVAVYYCLQSKNFPRTFGQGTKVEIKGSTSGSGKPGSGEGSTKGQIQLVQSGPELKKPGGSVKISCAASGYTFTSYDINWVRQAPGKGLEWVGWINTETREPAYAYDFRGRFTFSADTSKSTAYLQMNSLRAEDTAVYYCALDYLYSLDFWGQGTLVTVSS

SEQ ID NO: 61 - protein, 246 aa, synthetic construct
DIVLTQSPSSLSASVGDRATISCRASESVSFLGINLIHWYQQKPGQAPKLLIYLASNLETGVPARFSGSGSGTDFTLTISSVEPEDVAVYYCLQSRTLPRTFGQGTKVEIKGSTSGSGKPGSGEGSTKGQIQLVQSGPELKKPGGSVKISCAASGYTFTSYSINWVRQAPGKGLEWVGWINTETREPAYAQGFTGRFTFSADTSKSTAYLQMNSLRAEDTAVYYCALDYLYSLDFWGQGTLVTVSS

SEQ ID NO: 62 - protein, 246 aa, synthetic construct
DIVLTQSPSSLSASVGDRATISCRASESVTILGSHLIHWYQQKPGQAPKLLINLASNVNTGVPARFSGSGSGTDFTLTISSVEPEDVAVYYCLQSRTIPRTFGQGTKVEIKGSTSGSGKPGSGEGSTKGQIQLVQSGPELKKPGGSVKISCAASGYTFTDYSINWVRQAPGKGLEWVGWINTETREPAYAYDFRGRFTFSADTSKSTAYLQMNSLRAEDTAVYYCALDYSYAMDYWGQGTLVTVSS

SEQ ID NO: 63 - protein, 246 aa, synthetic construct
DIVLTQSPSSLSASVGDRATISCRASESVTILGSHLIHWYQQKPGQAPKLLIQLASNVQTGVPARFSGSGSGTDFTLTISSVEPEDVAVYYCLQSRTLPRTFGQGTKVEIKGSTSGSGKPGSGEGSTKGQIQLVQSGPELKKPGGSVKISCAASGYTFTDYSINWVRQAPGKGLEWVGWINTETREPAYAYDFRGRFTFSADTSKSTAYLQMNSLRAEDTAVYYCALDYSYAMDYWGQGTLVTVSS

SEQ ID NO: 64 - protein, 246 aa, synthetic construct
DIVLTQSPSSLSASVGDRATISCRASESVTILGSHLIHWYQQKPGQAPKLLIQLASNVQTGVPARFSGSGSGTDFTLTISSVEPEDVAVYYCLQSKNFPRTFGQGTKVEIKGSTSGSGKPGSGEGSTKGQIQLVQSGPELKKPGGSVKISCAASGYTFTDYSINWVRQAPGKGLEWVGWINTETREPAYAYDFRGRFTFSADTSKSTAYLQMNSLRAEDTAVYYCALDYSYAMDYWGQGTLVTVSS

SEQ ID NO: 65 - protein, 15 aa, synthetic construct
GGGGSGGGGSGGGGS

SEQ ID NO: 66 - protein, 5 aa, synthetic construct
GGGGS

SEQ ID NO: 67 - protein, 18 aa, synthetic construct
VEGGSGGSGGSGGSGGVD

SEQ ID NO: 68 - protein, 20 aa, synthetic construct
GGGGSGGGGSGGGGSGGGGS

SEQ ID NO: 69 - skipped

SEQ ID NO: 70 - protein, 4 aa, synthetic construct
AAAL

SEQ ID NO: 71 - protein, 18 aa, synthetic construct
GSTSGSGKPGSGEGSTKG

SEQ ID NO: 72 - protein, 31 aa, synthetic construct
SSGGSGGSGGSGGSGGSGGSGGSGGSGGSGS

SEQ ID NO: 73 - protein, 31 aa, synthetic construct
GSGGSGGSGGSGGSGGSGGSGGSGGSGGSGS

SEQ ID NO: 74 - protein, 608 aa, Homo sapiens
KWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 75 - protein, 531 aa, synthetic construct
MGWSCIILFLVATATGVHSDYKDDDDKDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 76 - protein, 519 aa, synthetic construct
MGWSCIILFLVATATGVHSDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 77 - protein, 521 aa, synthetic construct
MGWSCIILFLVATATGVHSDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLVESGGGVVQPGRSLRLSCSSSGYTETRYTMHWVRQAPGKGLEWIGINPSRGYTNYNQKVKDRFTISTDKSKSTAFLQMDSLRPEDTAVYYCARYYDDHYCLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRHHHHHH

SEQ ID NO: 78 - protein, 522 aa, synthetic construct
MGWSCIILFLVATATGVHSDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLVQSGAEVKKPGSSVKVSCKASGYKFTSYVMHWVRQAPGQGLEWMGYINPYNDVTKYNEKFKGRATITADESTNTAYMELSSLRSEDTAFYFCARGSYYDYDGFVYWGQGTLVTVSSGGGGSGGGGSGGGGSGGGGSDIQMTQSPSTLSASVGDRVTITCSATSSVSYMHWYQQKPGKAPKRLMYDTSKLASGVPARFIGSGSGTEFTLTISSLQPDDFATYYCQQWSSNPLTFGGGTKVEIKHHHHHH

SEQ ID NO: 79 - protein, 522 aa, synthetic construct
MGWSCIILFLVATATGVHSDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLVQSGAEVKKPGSSVKVSCKASGYKFTSYVMHWVKQAPGQGLEWIGYINPYNDVTKYNEKFKGKATLTADESTNTAYMELSSLRSEDTAVHYCARGSYYDYDGFVYWGQGTLVTVSSGGGGSGGGGSGGGGSGGGGSDIQMTQSPSTLSASVGDRVTMTCSATSSVSYMHWYQQKPGKAPKRWIYDTSKLASGVPARFIGSGSGTEFTLTISSLQPDDFATYYCQQWSSNPLTFGGGTKVEIKHHHHHH

SEQ ID NO: 80 - protein, 521 aa, synthetic construct
MGWSCIILFLVATATGVHSDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 81 - protein, 1131 aa, synthetic construct
MGWSCIILFLVATATGVHSDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSAASKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGLAAALQVQLVESGGGVVQPGRSLRLSCSSSGYTETRYTMHWVRQAPGKGLEWIGINPSRGYTNYNQKVKDRFTISTDKSKSTAFLQMDSLRPEDTAVYYCARYYDDHYCLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRHHHHHH

SEQ ID NO: 82 - protein, 1132 aa, synthetic construct
MGWSCIILFLVATATGVHSDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAASKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGLHHHHHH

SEQ ID NO: 83 - protein, 519 aa, synthetic construct
MGWSCIILFLVATATGVHSDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLVESGGGVVQPGRSLRLSCSSSGYTETRYTMHWVRQAPGKGLEWIGINPSRGYTNYNQKVKDRFTISTDKSKSTAFLQMDSLRPEDTAVYYCARYYDDHYCLDYWGQGTPVTVSSVEGGSGGSGGSGGSGGVDDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRHHHHHH

SEQ ID NO: 84 - protein, 522 aa, synthetic construct; identical to SEQ ID NO: 79

SEQ ID NO: 85 - protein, 518 aa, synthetic construct
MGWSCIILFLVATATGVHSDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGGGGSGGGGSGGGGSQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLVESGGGVVQPGRSLRLSCSSSGYTETRYTMHWVRQAPGKGLEWIGINPSRGYTNYNQKVKDRFTISTDKSKSTAFLQMDSLRPEDTAVYYCARYYDDHYCLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRHHHHHH

SEQ ID NO: 86 - protein, 1160 aa, synthetic construct
MGWSCIILFLVATATGVHSDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSSGGSGGSGGSGGSGGSGGSGGSGGSGGSGSKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGLHHHHHH

SEQ ID NO: 87 - protein, 1189 aa, synthetic construct
MGWSCIILFLVATATGVHSDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGSGGSGGSGGSGGSGGSGGSGGSGGSGGSGSKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGLSSGGSGGSGGSGGSGGSGGSGGSGGSGGSGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGSHHHHHH

SEQ ID NO: 88 - protein, 1061 aa, synthetic construct
MGWSCIILFLVATATGVHSDYKDDDDKDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSSGGSGGSGGSGGSGGGGSGGSGGSGGSGGSGSDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGSHHHHHH

SEQ ID NO: 89 - protein, 1087 aa, synthetic construct
MGWSCIILFLVATATGVHSDYKDDDDKQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGSTSGSGKPGSGEGSTKGDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKSSGGSGGSGGSGGSGGSGGSGGSGGSGGSGSDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSSGGSGGSGGSGGSGGGGSGGSGGSGGSGGSGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSGGGGSGGGGSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGSHHHHHH

SEQ ID NO: 90 - protein, 1115 aa, synthetic construct
MGWSCIILFLVATATGVHSDYKDDDDKQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGSTSGSGKPGSGEGSTKGDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKSSGGSGGSGGSGGSGGSGGSGGSGGSGGSGSDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSSSGGSGGSGGSGGSGGGGSGGSGGSGGSGGSGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSSGGSGGSGGSGGSGGGGSGGSGGSGGSGGSGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSGGGGSGGGGSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGSHHHHHH

SEQ ID NO: 91 - protein, 809 aa, synthetic construct
MGWSCIILFLVATATGVHSDYKDDDDKDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSSGGSGGSGGSGGSGGSGGSGGSGGSGGSGSDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGSHHHHHH

SEQ ID NO: 92 - protein, 809 aa, synthetic construct
MGWSCIILFLVATATGVHSDYKDDDDKDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSSGGSGGSGGSGGSGGSGGSGGSGGSGGSGSDIVLTQSPASLAVSPGQRATITCRASESVTILGSHLIHWYQQKPGQPPKLLINLASNVNTGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTDYSINWVRQAPGKGLEWVGWINTETREPAYAYDFTGRFTFSADTSKSMAYLQINSLRAEDTAVYYCALDYTYGMDYWGQGTLVTVSSGGSHHHHHH

SEQ ID NO: 93 - protein, 1090 aa, synthetic construct
MGWSCIILFLVATATGVHSDYKDDDDKQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGSTSGSGKPGSGEGSTKGDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKSSGGSGGSGGSGGSGGGGSGGSGGSGGSGGSGSDIVLTQSPASLAVSPGQRATITCRASESVTILGSHLIHWYQQKPGQPPKLLINLASNVNTGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTDYSINWVRQAPGKGLEWVGWINTETREPAYAYDFTGRFTFSADTSKSMAYLQINSLRAEDTAVYYCALDYTYGMDYWGQGTLVTVSSSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSSGGSGGSGGSGGSGGGGSGGSGGSGGSGGSGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSGGGGSGGGGSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGSHHHHHH

SEQ ID NO: 94 - protein, 1089 aa, synthetic construct
MGWSCIILFLVATATGVHSDYKDDDDKQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGSGGGGSGGGGSGGGGSDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKSSGGSGGSGGSGGSGGGGSGGSGGSGGSGGSGSDIVLTQSPASLAVSPGQRATITCRASESVTILGSHLIHWYQQKPGQPPKLLINLASNVNTGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTDYSINWVRQAPGKGLEWVGWINTETREPAYAYDFTGRFTFSADTSKSMAYLQINSLRAEDTAVYYCALDYTYGMDYWGQGTLVTVSSSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSSGGSGGSGGSGGSGGGGSGGSGGSGGSGGSGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKVEGGSGGSGGSGGSGGVDDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGSHHHHHH

SEQ ID NO: 95 - protein, 663 aa, synthetic construct
MGWSCIILFLVATATGVHSDYKDDDDKTRVDQTPRTATRETGESLTINCVLTDTSYPLYSTYWYRKNPGSSNKEQISISGRYVESVNKGTKSFSLRIKDLTVADSATYICRAMGTNIWTGDGAGTVLTVNSSGGSGGSGGSGGSGGSGGSGGSGGSGGSGSDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 96 - protein, 694 aa, synthetic construct
MGWSCIILFLVATATGVHSDYKDDDDKDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGSGGSGGSGGSGGSGGSGGSGGSGGSGGSGSTRVDQTPRTATRETGESLTINCVLTDTSYPLYSTYWYRKNPGSSNKEQISISGRYVESVNKGTKSFSLRIKDLTVADSATYICRAMGTNIWTGDGAGTVLTVNSSGGSGGSGGSGGSGGGGSGGSGGSGGSGGSGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGSHHHHHH

SEQ ID NO: 97 - protein, 663 aa, synthetic construct
MGWSCIILFLVATATGVHSDYKDDDDKDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSSGGSGGSGGSGGSGGSGGSGGSGGSGGSGSTRVDQTPRTATRETGESLTINCVLTDTSYPLYSTYWYRKNPGSSNKEQISISGRYVESVNKGTKSFSLRIKDLTVADSATYICRAMGTNIWTGDGAGTVLTVNHHHHHH

SEQ ID NO: 98 - protein, 792 aa, synthetic construct
MGWSCIILFLVATATGVHSDYKDDDDKDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSSGGSGGSGGSGGSGGSGGSGGSGGSGGSGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 99 - protein, 811 aa, synthetic construct
MGWSCIILFLVATATGVHSDYKDDDDKDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSSGGSGGSGGSGGSGGSGGSGGSGGSGGSGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGGGTKVEIKGGGGSGGGGSGGGGSGGGGSEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSHHHHHH

SEQ ID NO: 100 - protein, 842 aa, synthetic construct
MGWSCIILFLVATATGVHSDYKDDDDKDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGSGGSGGSGGSGGSGGSGGSGGSGGSGGSGSEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSGGGGSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGGGTKVEIKSSGGSGGSGGSGGSGGGGSGGSGGSGGSGGSGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSGGGGSGGGGSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGSHHHHHH

SEQ ID NO: 101 - protein, 793 aa, synthetic construct
MYRMQLLSCIALSLALVTNSDIVLTQSPASLAVSPGQRATISCRASESVSFLGINLIHWYQQKPGQPPKLLIYLASNLETGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLVQSGAEVKKPGASVKVSCKASGYTFTRYTMHWVRQAPGQGLEWMGYINPSKGYTNYNQKFKGRVTITADKSTSTAYMELSSLRSEDTAVYYCARYYDDHYSLDYWGQGTLVTVSSGGGGSGGGGSGGGGSGGGGSEIVMTQSPATLSVSPGERATLSCRASSSVSYLNWYQQKPGTAPKRWIYDTSKVASGVPYRFSGSGSGTDFTLTISSLQAEDEATYYCQQWSSNPLTFGQGTKLEIKSSGGSGGSGGSGGSGGSGGSGGSGGSGGSGSDIVLTQSPASLAVSPGQRATISCRASESVSFLGINLIHWYQQKPGQPPKLLIYLASNLETGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSS

SEQ ID NO: 102 - protein, 356 aa, synthetic construct
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 103 - protein, 196 aa, synthetic construct
NLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 104 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 105 - protein, 545 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 106 - RNA, 2658 nt, synthetic construct
  source 1..2658: mol_type = other RNA
agacccaagctggctagctctaaagaagcccctgggagcacagctcatcaccgccaccatgtacaggatgcaactcctgtcttgcattgcactaagtcttgcacttgtcacgaattcggacattgttttgactcaaagcccagcgagcttggccgtaagcccaggtcagcgagcaacaatatcttgccgagcttctgagtcagtatccttcctgggaatcaatttgatccattggtatcaacaaaagccagggcaacccccgaaactgcttatatacctggcctcaaatttggagaccggggtcccggcacgattttcagggtcaggttctgggaccgactttacgcttacgatttcttccgtcgaaccggaggacaccgctaactattattgtttgcaatcccggacccttccacgaacatttggccaaggaacaaaagttgaaatcaaaggttccacttcaggatctggcaagcctggcagcggagaaggctcaacgaagggccagatacagctcgtccagagtggccccgagctgaaaaaaccgggtggcagtgttaaaatcagttgtaaagcgagcggatacacgttcacgtcatactccattaactgggttcggcaagcaccaggaaagggtcttgaatgggtcgggtggatcaacaccgagacaagagaacctgcctatgcccaagggttcactgggaggttcacattctctgctgatacctccaagtctatggcgtacttgcaaattaatagtctcagagccgaggacactgctgtctattactgtgcactcgactacctctatagtctggacttttggggtcagggtactttggtcacggtctcttctggaggtggtggatcccaagttcagttggtccagtctggtgccgaggttaaaaaaccaggtgcatctgtcaaagtgagttgcaaagcatcaggctataccttcacaaggtacacaatgcattgggtgaggcaggcccctgggcagggcctcgaatggatgggatacatcaacccttccaaaggttacaccaactacaatcagaaatttaagggtcgcgtcaccattactgcagataaatctacaagcacagcatatatggaattgtcatctctccgctccgaggataccgccgtctattactgtgcaaggtactacgatgaccactatagtcttgattactgggggcaagggacattggttacagtgagctccggaggcggaggatctggtggtggcggatctggcggcggtggaagtggcggaggtggtagcgagatcgtgatgacccagtcacccgctaccttgtctgtctcaccaggcgagagagccactcttagttgtagggcctccagttcagtcagctatctcaactggtatcagcagaagcccggcacagcccccaagcggtggatttatgacacaagtaaggtggccagtggggtcccttaccggttcagcggttccggtagtggcactgatttcacactcaccatttcttcacttcaagcagaagatgaggcaacttactactgccagcaatggagttccaatccccttacattcggtcaggggacaaaactggagattaagtctagcggaggctctggtggatctggcggaagcggaggcagtggtggatccggaggtagcggcggatctggcggtagtggaggtagtggatctgatatcgtactcacacagagtcctgcttccctcgcggtatcacctggacaaagggccaccatcagctgtagggctagtgaaagtgtttcatttctcgggataaacctgattcactggtaccagcagaaacctggacagccacctaaactgctgatctatcttgcaagcaacctcgaaacaggagtaccagctaggttcagcggcagcggatccggcactgatttcactctcaccatatcctctgttgagccagaagatacagcaaattactactgcctccagagtcgcactctgcctaggactttcgggcaggggaccaaggtcgagattaaggggagtacgagtggtagcggtaaaccaggatctggcgagggatccaccaaaggacaaatccaattggttcaaagcggacctgaactcaagaagccaggagggtcagtgaagatttcctgcaaggctagtgggtatacatttacctcctatagcatcaattgggtacgacaggctccggggaaaggtttggagtgggtgggctggattaatactgaaactcgcgagcccgcatacgctcagggatttacgggtcgctttaccttttcagcggacacaagtaaaagcatggcatacctccagatcaactcactgcgcgcggaagataccgcggtgtactattgcgccctggattatctttactctctcgatttctggggccaaggcacgctcgtgacagtatcctcctagctatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagc

SEQ ID NO: 107 - RNA, 1917 nt, synthetic construct
  source 1..1917: mol_type = other RNA
agacccaagctggctagctctaaagaagcccctgggagcacagctcatcaccgccgccaccatgtgtcatcaacaactggtaatttcatggttttcactcgtattcctggcatcacctcttgttgcgatctgggaactgaagaaggatgtctacgttgtcgagttggattggtatcctgatgccccaggtgagatggttgtgttgacttgtgatacacctgaagaggacggtattacttggacactcgaccagtcctccgaggtcttgggcagcggtaaaacccttaccatccaggtgaaggagttcggagatgctggacagtacacatgtcataaggggggcgaagtcctgtctcactccctcttgctgttgcataaaaaggaggacgggatctggtctacagatattcttaaagaccaaaaggaacccaaaaacaaaacattcctcagatgcgaagccaaaaattactccggtagatttacatgttggtggcttacaacaatctccacagacctcacattctccgtcaaaagctccagaggatcaagcgatccacagggagtcacttgcggcgctgcaacactctccgcagagagagtgcgcggcgacaataaggaatacgaatactctgttgagtgtcaagaggatagtgcttgtccagcagccgaagaatccctgccaatcgaagtcatggtggacgctgtccacaagctgaaatacgaaaattacaccagtagtttctttatccgcgacatcattaagcccgaccctccaaagaatcttcagcttaaaccactcaagaactcaagacaggttgaagtctcatgggaatatccagatacatggtcaacccctcattcttatttcagtcttactttttgtgtgcaggttcaaggaaagtcaaagagagagaagaaagacagagtttttactgataaaacaagtgcaacagtgatctgtaggaagaatgcaagcattagtgtccgggctcaagacaggtactatagtagctcttggtcagagtgggcatcagtgccatgttcaggcggaggcggaagcggcggaggcggatccggaggcggaggcagcggaggcggaggctccaggaacttgcccgttgcaactcccgatcccggtatgttcccttgcctgcatcactctcaaaacttgttgcgggctgtttctaacatgctccagaaggcaaggcagacactggaattttacccttgcacatccgaagagatcgaccacgaggacattacaaaagataaaactagcactgttgaggcctgcttgccccttgagttgaccaaaaacgagtcctgtctgaactcacgggaaacatccttcatcactaatgggagctgtttggcttcaagaaaaacatccttcatgatggccctttgcttgagctctatctacgaggacttgaaaatgtaccaggtcgagttcaagacaatgaacgcaaaactgttgatggaccccaagagacaaattttcctggatcaaaatatgttggccgttattgacgagctgatgcaagcactgaactttaacagtgaaacagtgcctcagaaatcttccctggaggaaccagacttttacaagaccaaaatcaaattgtgcattctcctgcacgccttccggatcagagcagtgactattgaccgcgtcatgtcttatctgaacgcaagttgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagc

SEQ ID NO: 108 - RNA, 1350 nt, synthetic construct
  source 1..1350: mol_type = other RNA
agacccaagctggctagctctaaagaagcccctgggagcacagctcatcaccgccgccaccatgtccattcctttgcctcttttgcagatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagc

SEQ ID NO: 109 - protein, 19 aa, synthetic construct
MGWSCIILFLVATATGVHS

SEQ ID NO: 110 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 111 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 112 - protein, 118 aa, synthetic construct
QVQLVESGGGVVQPGRSLRLSCSSSGYTETRYTMHWVRQAPGKGLEWIGINPSRGYTNYNQKVKDRFTISTDKSKSTAFLQMDSLRPEDTAVYYCARYYDDHYCLDYWGQGTPVTVSS

SEQ ID NO: 113 - protein, 107 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 114 - protein, 120 aa, synthetic construct
QVQLVQSGAEVKKPGSSVKVSCKASGYKFTSYVMHWVRQAPGQGLEWMGYINPYNDVTKYNEKFKGRATITADESTNTAYMELSSLRSEDTAFYFCARGSYYDYDGFVYWGQGTLVTVSS

SEQ ID NO: 115 - protein, 106 aa, synthetic construct
DIQMTQSPSTLSASVGDRVTITCSATSSVSYMHWYQQKPGKAPKRLMYDTSKLASGVPARFIGSGSGTEFTLTISSLQPDDFATYYCQQWSSNPLTFGGGTKVEIK

SEQ ID NO: 116 - protein, 120 aa, synthetic construct
QVQLVQSGAEVKKPGSSVKVSCKASGYKFTSYVMHWVKQAPGQGLEWIGYINPYNDVTKYNEKFKGKATLTADESTNTAYMELSSLRSEDTAVHYCARGSYYDYDGFVYWGQGTLVTVSS

SEQ ID NO: 117 - protein, 106 aa, synthetic construct
DIQMTQSPSTLSASVGDRVTMTCSATSSVSYMHWYQQKPGKAPKRWIYDTSKLASGVPARFIGSGSGTEFTLTISSLQPDDFATYYCQQWSSNPLTFGGGTKVEIK

SEQ ID NO: 118 - protein, 119 aa, synthetic construct
QVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS

SEQ ID NO: 119 - protein, 106 aa, synthetic construct
QIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 120 - RNA, 132 nt, synthetic construct
  source 1..132: mol_type = other RNA
tctcaacacaacatatacaaaacaaacgaatctcaagcaatcaagcattctacttctattgcagcaatttaaatcatttcttttaaagcaaaagcaattttctgaaaattttcaccatttacgaacgatagc

SEQ ID NO: 121 - RNA, 278 nt, synthetic construct
  source 1..278: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 122 - protein, 103 aa, synthetic construct
TRVDQTPRTATRETGESLTINCVLTDTSYPLYSTYWYRKNPGSSNKEQISISGRYVESVNKGTKSFSLRIKDLTVADSATYICRAMGTNIWTGDGAGTVLTVN

SEQ ID NO: 123 - protein, 121 aa, synthetic construct
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSS

SEQ ID NO: 124 - protein, 110 aa, synthetic construct
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGGGTKVEIK

SEQ ID NO: 125 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - RNA, 52 nt, synthetic construct
  source 1..52: mol_type = other RNA
agacccaagctggctagctctaaagaagcccctgggagcacagctcatcacc

SEQ ID NO: 127 - RNA, 58 nt, synthetic construct
  source 1..58: mol_type = other RNA
agacccaagctggctagctctaaagaagcccctgggagcacagctcatcaccgccacc

SEQ ID NO: 128 - RNA, 218 nt, synthetic construct
  source 1..218: mol_type = other RNA
ctatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagc

SEQ ID NO: 129 - protein, 793 aa, synthetic construct
MYRMQLLSCIALSLALVTNSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSSGGSGGSGGSGGSGGSGGSGGSGGSGGSGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 130 - protein, 793 aa, synthetic construct
MYRMQLLSCIALSLALVTNSDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLVQSGAEVKKPGASVKVSCKASGYTFTRYTMHWVRQAPGQGLEWIGYINPSKGYTNYNQKFKGRVTMTADKSTSTAYMELSSLRSEDTAVYYCARYYDDHYSLDYWGQGTLVTVSSGGGGSGGGGSGGGGSGGGGSEIVMTQSPATLSLSPGERVTLSCRASSSVSYMNWYQQKSGTSPKRWIYDTSKVASGVPYRFSGSGSGTDFTLTISSLQAEDEATYYCQQWSSNPLTFGQGTKLEIKSSGGSGGSGGSGGSGGSGGSGGSGGSGGSGSDIVLTQSPASLAVSPGQRATITCRASESVTILGSHLIHWYQQKPGQPPKLLINLASNVNTGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTDYSINWVRQAPGKGLEWVGWINTETREPAYAYDFTGRFTFSADTSKSMAYLQINSLRAEDTAVYYCALDYTYGMDYWGQGTLVTVSS

SEQ ID NO: 131 - protein, 793 aa, synthetic construct
MYRMQLLSCIALSLALVTNSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSQVQLVQSGAEVKKPGASVKVSCKASGYTFTRYTMHWVRQAPGQGLEWIGYINPSKGYTNYNQKFKGRVTMTADKSTSTAYMELSSLRSEDTAVYYCARYYDDHYSLDYWGQGTLVTVSSGGGGSGGGGSGGGGSGGGGSEIVMTQSPATLSLSPGERVTLSCRASSSVSYMNWYQQKSGTSPKRWIYDTSKVASGVPYRFSGSGSGTDFTLTISSLQAEDEATYYCQQWSSNPLTFGQGTKLEIKSSGGSGGSGGSGGSGGSGGSGGSGGSGGSGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 132 - protein, 516 aa, synthetic construct
MYRMQLLSCIALSLALVTNSDIVLTQSPASLAVSPGQRATITCRASESVTILGSHLIHWYQQKPGQPPKLLINLASNVNTGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTDYSINWVRQAPGKGLEWVGWINTETREPAYAYDFTGRFTFSADTSKSMAYLQINSLRAEDTAVYYCALDYTYGMDYWGQGTLVTVSSGGGGSQVQLVQSGAEVKKPGASVKVSCKASGYTFTRYTMHWVRQAPGQGLEWMGYINPSKGYTNYNQKFKGRVTITADKSTSTAYMELSSLRSEDTAVYYCARYYDDHYSLDYWGQGTLVTVSSGGGGSGGGGSGGGGSGGGGSEIVMTQSPATLSVSPGERATLSCRASSSVSYLNWYQQKPGTAPKRWIYDTSKVASGVPYRFSGSGSGTDFTLTISSLQAEDEATYYCQQWSSNPLTFGQGTKLEIK

SEQ ID NO: 133 - protein, 793 aa, synthetic construct
MYRMQLLSCIALSLALVTNSDIVLTQSPASLAVSPGQRATITCRASESVSFLGINLIHWYQQKPGQPPKLLIYSASNLQSGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLVQSGAEVKKPGASVKVSCKASGYTFTRYTMHWVRQAPGQGLEWMGYINPSKGYTNYNQKFKGRVTITADKSTSTAYMELSSLRSEDTAVYYCARYYDDHYSLDYWGQGTLVTVSSGGGGSGGGGSGGGGSGGGGSEIVMTQSPATLSVSPGERATLSCRASSSVSYLNWYQQKPGTAPKRWIYDTSKVASGVPYRFSGSGSGTDFTLTISSLQAEDEATYYCQQWSSNPLTFGQGTKLEIKSSGGSGGSGGSGGSGGSGGSGGSGGSGGSGSDIVLTQSPASLAVSPGQRATITCRASESVTILGSHLIHWYQQKPGQPPKLLINLASNVNTGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTDYSINWVRQAPGKGLEWVGWINTETREPAYAYDFTGRFTFSADTSKSMAYLQINSLRAEDTAVYYCALDYTYGMDYWGQGTLVTVSS

SEQ ID NO: 134 - protein, 793 aa, synthetic construct
MYRMQLLSCIALSLALVTNSDIVLTQSPASLAVSPGQRATITCRASESVTILGSHLIHWYQQKPGQPPKLLINLASNVNTGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTDYSINWVRQAPGKGLEWVGWINTETREPAYAYDFTGRFTFSADTSKSMAYLQINSLRAEDTAVYYCALDYTYGMDYWGQGTLVTVSSGGGGSQVQLVQSGAEVKKPGASVKVSCKASGYTFTRYTMHWVRQAPGQGLEWMGYINPSKGYTNYNQKFKGRVTITADKSTSTAYMELSSLRSEDTAVYYCARYYDDHYSLDYWGQGTLVTVSSGGGGSGGGGSGGGGSGGGGSEIVMTQSPATLSVSPGERATLSCRASSSVSYLNWYQQKPGTAPKRWIYDTSKVASGVPYRFSGSGSGTDFTLTISSLQAEDEATYYCQQWSSNPLTFGQGTKLEIKSSGGSGGSGGSGGSGGSGGSGGSGGSGGSGSDIVLTQSPASLAVSPGQRATITCRASESVTILGSHLIHWYQQKPGQPPKLLINLASNVNTGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTDYSINWVRQAPGKGLEWVGWINTETREPAYAYDFTGRFTFSADTSKSMAYLQINSLRAEDTAVYYCALDYTYGMDYWGQGTLVTVSS

SEQ ID NO: 135 - protein, 793 aa, synthetic construct; identical to SEQ ID NO: 101

SEQ ID NO: 136 - protein, 793 aa, synthetic construct
MYRMQLLSCIALSLALVTNSDIVLTQSPASLAVSPGQRATITCRASESVTILGSHLIHWYQQKPGQPPKLLINLASNVNTGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTDYSINWVRQAPGKGLEWVGWINTETREPAYAYDFTGRFTFSADTSKSMAYLQINSLRAEDTAVYYCALDYTYGMDYWGQGTLVTVSSGGGGSQVQLVQSGAEVKKPGASVKVSCKASGYTFTRYTMHWVRQAPGQGLEWIGYINPSKGYTNYNQKFKGRVTMTADKSTSTAYMELSSLRSEDTAVYYCARYYDDHYSLDYWGQGTLVTVSSGGGGSGGGGSGGGGSGGGGSEIVMTQSPATLSLSPGERVTLSCRASSSVSYMNWYQQKSGTSPKRWIYDTSKVASGVPYRFSGSGSGTDFTLTISSLQAEDEATYYCQQWSSNPLTFGQGTKLEIKSSGGSGGSGGSGGSGGSGGSGGSGGSGGSGSDIVLTQSPASLAVSPGQRATITCRASESVTILGSHLIHWYQQKPGQPPKLLINLASNVNTGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTDYSINWVRQAPGKGLEWVGWINTETREPAYAYDFTGRFTFSADTSKSMAYLQINSLRAEDTAVYYCALDYTYGMDYWGQGTLVTVSS

SEQ ID NO: 137 - protein, 793 aa, synthetic construct
MYRMQLLSCIALSLALVTNSDIVLTQSPASLAVSPGQRATISCRASESVSFLGINLIHWYQQKPGQPPKLLIYLASNLETGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSSGGGGSQVQLVQSGAEVKKPGASVKVSCKASGYTFTRYTMHWVRQAPGQGLEWIGYINPSKGYTNYNQKFKGRVTMTADKSTSTAYMELSSLRSEDTAVYYCARYYDDHYSLDYWGQGTLVTVSSGGGGSGGGGSGGGGSGGGGSEIVMTQSPATLSLSPGERVTLSCRASSSVSYMNWYQQKSGTSPKRWIYDTSKVASGVPYRFSGSGSGTDFTLTISSLQAEDEATYYCQQWSSNPLTFGQGTKLEIKSSGGSGGSGGSGGSGGSGGSGGSGGSGGSGSDIVLTQSPASLAVSPGQRATISCRASESVSFLGINLIHWYQQKPGQPPKLLIYLASNLETGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSS

SEQ ID NO: 138 - protein, 793 aa, synthetic construct; identical to SEQ ID NO: 137

SEQ ID NO: 139 - protein, 20 aa, Homo sapiens
MYRMQLLSCIALSLALVTNS

SEQ ID NO: 140 - protein, 246 aa, synthetic construct
DIVLTQSPASLAVSPGQRATISCRASESVSFLGINLIHWYQQKPGQPPKLLIYLASNLETGVPARFSGSGSGTDFTLTISSVEPEDTANYYCLQSRTLPRTFGQGTKVEIKGSTSGSGKPGSGEGSTKGQIQLVQSGPELKKPGGSVKISCKASGYTFTSYSINWVRQAPGKGLEWVGWINTETREPAYAQGFTGRFTFSADTSKSMAYLQINSLRAEDTAVYYCALDYLYSLDFWGQGTLVTVSS

SEQ ID NO: 141 - protein, 29 aa, synthetic construct
GGSLEQKLISEEDLAANDILDYKDDDDKV